(19) United States
(12) Patent Application Publication
(10) Pub. No.: US 2026/0060858 A1
(43) Pub. Date: Mar. 5, 2026

(54) PATIENT SUPPORT APPARATUS WITH OBSTRUCTION DETECTION

(71) Applicant: Stryker Corporation, Portage, MI (US)

(72) Inventors: Michael W. Graves, Augusta, MI (US); Lavanya Vytla, Prosper, TX (US); Anish Paul, Portage, MI (US); Krishna Sandeep Bhimavarapu, Kalamazoo, MI (US); Celso Henrique Farnese Pires Pereira, Portage, MI (US); Madhu Sandeep Thota, Portage, MI (US); Matthew A. Cutler, Mattawan, MI (US); Tyler Joseph Ethen, Portage, MI (US); Brianna R. Graves, Paw Paw, MI (US)

(21) Appl. No.: 19/103,646

(22) PCT Filed: Aug. 15, 2023
(86) PCT No.: PCT/US2023/030190

§ 371 Date: Feb. 13, 2025

Related U.S. Application Data
(60) Provisional application No. 63/398,088, filed on Aug. 15, 2022.
(60) Provisional application No. 63/413,804, filed on Oct. 6, 2022.

(51) Int. Cl. A61G 7/005; A61G 7/018; A61G 7/05

(52) U.S. Cl. CPC A61G 7/005; A61G 7/018; A61G 7/0524; A61G 7/0527; A61G 2203/20; A61G 2203/32; A61G 2203/44; A61G 2203/72

(57) ABSTRACT

A patient support apparatus, such as a bed, cot, stretcher, etc., includes a frame, a plurality of force sensors adapted to detect a weight supported by the frame, an actuator, a control panel, and a controller. The controller is adapted to detect if a change in the weight exceeds a threshold amount while the actuator is moving a component of the patient support apparatus, to provide an indication to a user that contact with an obstruction has been made if the change in the weight exceeds the threshold amount, and to determine a location of the contact on the patient support apparatus. The controller may alternatively, or additionally, allow a user to change a value of the threshold, to display a recovery control on a touch screen, to use different threshold amounts for different locations, and/or take other actions in response to, and/or regarding, detecting an obstruction.

### CROSS-REFERENCE TO RELATED APPLICATIONS

This application claims priority to U.S. provisional patent application Ser. No. 63/398,088 filed Aug. 15, 2022, by inventors Michael Graves et al, and entitled PATIENT SUPPORT APPARATUS WITH OBSTRUCTION DETECTION, and to U.S. provisional patent application Ser. No. 63/413,804 filed Oct. 6, 2022, by inventors Michael Graves et al, and entitled PATIENT SUPPORT APPARATUS WITH OBSTRUCTION DETECTION, the complete disclosures of both of which are incorporated herein by reference.

### BACKGROUND

The present disclosure relates to patient support apparatuses, such as beds, cots, stretchers, recliners, or the like. More specifically, the present disclosure relates to patient support apparatuses that include a plurality of force sensors.

Existing hospital beds may include an obstruction detection sensor that automatically detects when movement of a component of the patient support apparatus causes the component to hit an obstruction. In some existing hospital beds, the obstruction detection sensors may include switches, switch plates, and/or other types of sensors that make physical contact with the obstruction. In other existing hospital beds, the obstruction detection sensor may comprise infrared emitters and sensors that detect an obstruction when the obstruction physically interferes with one or more beams of the emitted infrared light. In both of these types of obstruction detection sensors, obstructions are only detected in the location(s) where a physical detector is positioned on the hospital bed, or in location(s) where an infrared light beam is present. Contact with an obstruction outside of these locations is therefore not detected.

### SUMMARY

According to various embodiments, an improved patient support apparatus is provided that does not rely on physical contact with a sensor, or physical interference with an electromagnetic beam (e.g. an infrared light beam) in order to automatically detect when the patient support apparatus makes contact with an obstruction. In some embodiments, an improved patient support apparatus is provided that is capable of detecting contact with one or more obstructions over substantially all areas of the patient support apparatus that move. According to other aspects of the present disclosure, a patient support apparatus is provided that enables a user to select sensitivity levels for the detection of obstruction, that provides automatic assistance in moving the bed out of contact with an obstruction, that automatically suggests improvements in the sensitivity level to reduce the occurrence of false obstruction detections, that automatically takes other steps to reduce the occurrence of false obstruction detections, that determines the location of the point of contact of the obstruction on the patient support apparatus, and/or that performs other actions with respect to the detection and/or mitigation of obstructions to the movement of various components of the patient support apparatus. The obstruction detection system of the present disclosure may utilize the same force sensors built into the patient support apparatus that are used for the patient support apparatus's scale system and/or exit detection system, thereby eliminating the need to purchase additional sensors when implementing the obstruction detection system. Still other features and aspects of the present disclosure will be apparent to those skilled in the art in light of the accompanying drawing and the following written description.

According to a first aspect of the present disclosure, a patient support apparatus is provided that includes a support deck, a frame, a plurality of force sensors, an actuator, a control panel, and a controller. The support deck is adapted to support a patient thereon. The frame is adapted to support the support deck. The plurality of force sensors are adapted to detect a weight supported by the frame. The actuator is adapted to move a component of the patient support apparatus. The control panel includes a movement control that is adapted to drive the actuator when the movement control is activated. The controller is adapted to detect if a change in the weight exceeds a threshold amount while the actuator is moving the component, to provide an indication to a user that contact with an obstruction has been made if the change in the weight exceeds the threshold amount, and to determine a location of the contact on the patient support apparatus.

According to another aspect of the present disclosure, a patient support apparatus is provided that includes a support deck, a frame, a plurality of force sensors, an actuator, a control panel, and a controller. The support deck is adapted to support a patient thereon. The frame is adapted to support the support deck. The plurality of force sensors are adapted to detect a weight supported by the frame. The actuator is adapted to move a component of the patient support apparatus. The control panel includes a movement control adapted to drive the actuator when the movement control is activated. The controller is adapted to detect if a change in the weight exceeds a threshold amount while the actuator is moving the component, to provide an indication to a user that contact with an obstruction has been made if the change in the weight exceeds the threshold amount, and to allow the user to change a value of the threshold amount.

According to another aspect of the present disclosure, a patient support apparatus is provided that includes a support deck, a frame, a plurality of force sensors, an actuator, a control panel, and a controller. The support deck is adapted to support a patient thereon. The frame is adapted to support the support deck. The plurality of force sensors are adapted to detect a weight supported by the frame. The actuator is adapted to move a component of the patient support apparatus. The control panel includes a movement control adapted to drive the actuator when the movement control is activated. The controller is adapted to detect a location of a change in weight on the patient support apparatus while the actuator is moving the component. The controller is further adapted to provide an indication to a user that contact with an obstruction has been made if the change in the weight exceeds a first threshold amount and the change in weight occurs in a first area of the patient support apparatus. The controller is still further adapted to provide the indication to the user that contact with the obstruction has been made if the change in weight exceeds a second threshold amount and the change in weight occurs in a second area different from the first area. The first threshold amount is different from the second threshold amount.

According to another aspect of the present disclosure, a patient support apparatus is provided that includes a support deck, a frame, a plurality of force sensors, a plurality of actuators, a control panel, a display, and a controller. The support deck is adapted to support a patient thereon. The frame is adapted to support the support deck. The plurality of force sensors are adapted to detect a weight supported by the frame. The plurality of actuators are adapted to move one or more components of the patient support apparatus. The control panel includes a plurality of movement controls that are adapted to drive one or more of the actuators when the respective movement control is activated. The controller is adapted to detect if a change in the weight exceeds a threshold amount while one or more of the actuators is moving one or more of the components, to automatically stop movement of the one or more actuators if the change in the weight exceeds the threshold amount, and to automatically to display a recovery control on the display if the change in the weight exceeds the threshold amount. The recovery control, when activated, is adapted to move one or more of the components out of contact with an obstruction.

According to another aspect of the present disclosure, a patient support apparatus is provided that includes a support deck, a frame, a plurality of force sensors, an actuator, a control panel, and a controller. The support deck is adapted to support a patient thereon. The frame is adapted to support the support deck. The plurality of force sensors are adapted to detect a weight supported by the frame. The actuator is adapted to move a component of the patient support apparatus. The control panel includes a movement control adapted to drive the actuator when the movement control is activated. The controller is adapted to determine if the component has made contact with an obstruction while the component is moving by performing the following: detecting if a change in the weight exceeds a threshold amount while the actuator is moving the component, and analyzing a rate of the change in weight while the component is moving.

According to another aspect of the present disclosure, a patient support apparatus is provided that includes a support deck, a frame, a plurality of force sensors, an actuator, a control panel, and a controller. The support deck is adapted to support a patient thereon. The frame is adapted to support the support deck. The plurality of force sensors are adapted to detect a weight supported by the frame. The actuator is adapted to move a component of the patient support apparatus. The control panel includes a movement control adapted to drive the actuator when the movement control is activated. The controller is adapted to perform the following: to detect if a change in the weight exceeds a threshold amount while the actuator is moving the component, to automatically stop movement of the component if the change in the weight exceeds the threshold amount, to determine a maximum weight change reading while the component is moving and a residual weight change reading when the controller automatically stops movement of the component, and to automatically restart movement of the component if the maximum weight change reading and the residual weight change reading indicate that no contact was actually made with an obstruction.

According to another aspect of the present disclosure, a patient support apparatus is provided that includes a support deck, a frame, a plurality of force sensors, an actuator, a control panel, and a controller. The support deck is adapted to support a patient thereon. The frame is adapted to support the support deck. The plurality of force sensors are adapted to detect a weight supported by the frame. The actuator is adapted to move a component of the patient support apparatus in a first direction and a second direction opposite the first direction. The control panel includes a first movement control adapted to drive the actuator such that the component moves in the first direction when the first movement control is activated. The control panel also includes a second movement control adapted to drive the actuator such that the component moves in the second direction when the second movement control is activated. The controller is adapted to detect if a change in the weight exceeds a threshold amount while the actuator is moving the component in the first direction, to automatically stop movement of the component in the first direction if the change in the weight exceeds the threshold amount, to subsequently move the component in the second direction if a user activates the second movement control, and to not automatically stop movement of the component in the second direction for a predetermined time period, even if the controller detects a second change in the weight during the predetermined time period that exceeds the threshold amount.

According to another aspect of the present disclosure, a patient support apparatus is provided that includes a support deck, a frame, a plurality of force sensors, an actuator, a control panel, and a controller. The support deck is adapted to support a patient thereon. The frame is adapted to support the support deck. The plurality of force sensors are adapted to detect a weight supported by the frame. The actuator is adapted to move a component of the patient support apparatus. The control panel includes a movement control adapted to drive the actuator when the movement control is activated. The controller is adapted to automatically stop movement of the component in response to contact with an obstruction, to record a first change in weight during movement of the component and a residual change in weight when movement of the component is automatically stopped, and to use the first change in weight and the residual change in weight to determine if actual contact with the obstruction has occurred or a false detection of contact with the obstruction has occurred.

According to yet another aspect of the present disclosure, a patient support apparatus is provided that includes a support deck, a frame, a plurality of force sensors, a sensor, an actuator, a control panel, and a controller. The support deck is adapted to support a patient thereon and the frame is adapted to support the support deck. The plurality of force sensors are adapted to detect a weight supported by the frame. The sensor is adapted to wirelessly detect a tag. The actuator is adapted to move a component of the patient support apparatus. The control panel includes a movement control adapted to drive the actuator when the movement control is activated. The controller is adapted to detect a location of a change in weight on the patient support apparatus while the actuator is moving the component, to determine a position of the tag relative to the patient support apparatus, to select a zone based on the position of the tag relative to the patient support apparatus, and to stop movement of the component if the change in the weight exceeds a first threshold amount and the change in weight occurs in the selected zone.

According to other aspects of the present disclosure, the plurality of force sensors are load cells adapted to support the frame.

In some aspects, the control panel is adapted to allow the user to change a value of the threshold amount.

The control panel, in some aspects, is adapted to allow the user to select at least three different values for the threshold amount, wherein the at least three different values correspond to high, medium, and low sensitivity levels for detecting contact with the obstruction.

The controller, in some aspects, is adapted to determine the location of the contact with the obstruction by calculating a center of gravity of the change in the weight.

The controller, in some aspects, is further adapted to use a first value for the threshold amount if the location of the contact falls within a first area and to use a second value for the threshold amount if the location of the contact falls within a second area, wherein the first value is different from the second value and the first area is different from the second area.

The patient support apparatus, in some aspects, includes a plurality of siderails moveable between raised and lowered positions, and the controller is further adapted to define a boundary of the first area based on a position of at least one of the siderails.

In some aspects, the controller is adapted to define a boundary of the first area based on a position of each of the plurality of siderails.

The first area, in some aspects, includes an area between a head end and a foot end of the patient support apparatus and excludes both the head end and the foot end of the patient support apparatus.

In some aspects, the first value is greater than the second value.

The controller, in some aspects, is adapted to automatically stop movement of the component when contact is detected.

The controller, in some aspects, is adapted to determine a maximum weight change reading while the component is moving and a residual weight change reading when the controller automatically stops movement of the component.

In some aspects, the controller is adapted to use the maximum weight change reading and the residual weight change reading to evaluate whether to provide the indication to the user that contact with the obstruction has been made.

In some aspects, the controller is adapted to automatically restart movement of the component if the controller determines from the maximum weight change reading and the residual weight change reading that no actual contact with the obstruction has been made.

The controller, in some aspects, is adapted to determine a rate at which the weight changes while the component is moving.

The controller, in some aspects, is adapted to use the rate at which the weight changes to evaluate whether to provide the indication to the user that contact with the obstruction has been made.

In some aspects, the controller is adapted to automatically restart movement of the component if the controller determines from the rate at which the weight changes that no actual contact with the obstruction has been made.

The component, in some aspects, includes the frame and/or one or more pivotable sections of the support deck.

In some aspects, the controller is adapted to display the location of the contact with the obstruction on the display.

The controller, in some aspects, is adapted to detect if a second change in the weight exceeds a second threshold while no components are moving.

The controller, in some aspects, is further adapted to provide an indication to the user that an object has been added or removed from the patient support apparatus if the second change in the weight exceeds the second threshold.

In some aspects, the controller is adapted to determine a location of the object that has been added or removed from the patient support apparatus. The controller may determine the location of the object by calculating a center of gravity of the second change in the weight.

In some aspects, the controller is adapted to analyze outputs from the force sensors generated while the component is moving in order to determine if actual contact of the component with the obstruction has occurred or a false detection of contact with an obstruction has occurred.

In some aspects, the controller is adapted to record instances in which a false detection of contact has occurred, to analyze the instances, and to suggest a different threshold amount to the user in order to reduce a number of the instances in which actual contact has not occurred.

The movement control, in some aspects, is adapted to drive a plurality of actuators when the movement control is activated.

The control panel, in some aspects, includes a touch screen display and the controller is adapted to display a recovery control on the touch screen display in response to the contact with the obstruction. The recovery control is adapted to move the component out of contact with the obstruction when the recovery control is activated.

In some aspects, the controller is adapted to detect if a second change in the weight exceeds the threshold amount while the recovery control is activated, and to not provide the indication to the user if the second change in the weight is substantially equal to, but of opposite sign of, the change in weight.

The actuator, in some aspects, includes a head end actuator and a foot end actuator. The head end actuator is adapted to change a height of a head end of the patient support apparatus, and the foot end actuator is adapted to change a height of a foot end of the patient support apparatus.

In some aspects, the control panel includes a plurality of movement controls adapted to drive both the head end actuator and the foot end actuator in a plurality of coordinated manners, and the recovery control is adapted to drive both the head end actuator and the foot end actuator in a coordinated manner different from each one of the plurality of coordinated manners.

In some aspects, the controller is adapted to determine that no actual contact with the obstruction has been made if the change in weight exceeds the threshold amount but the rate of the change in weight does not meet a criteria.

The controller, in some aspects, is adapted to stop movement of the component if the change in weight exceeds a second threshold amount and the change in weight occurs outside of the selected zone.

In some aspects, the second threshold amount is smaller than the first threshold amount.

The tag, in some aspects, is adapted to be worn by a person.

The sensor, in some aspects, is a first ultra-wideband transceiver adapted to communicate with a second ultra-wideband transceiver incorporated into the tag.

In some aspects, the controller is adapted to determine a distance of the tag from the patient support apparatus, and the zone is defined as a distance from the patient support apparatus.

In some aspects, the sensor is one of a plurality of ultra-wideband transceivers adapted to communicate with a second ultra-wideband transceiver incorporated into the tag, the controller is further adapted to determine a three-dimensional position of the tag relative to the patient support apparatus, and the zone is defined in three dimensions.

In some aspects, the sensor is one of a plurality of ultra-wideband transceivers adapted to communicate with a second ultra-wideband transceiver incorporated into the tag, the controller is further adapted to determine a two-dimensional position of the tag relative to the patient support apparatus, and the zone is defined in two dimensions.

The zone, in some aspects, is one of multiple predetermined zones whose locations remain static as the tag moves.

The controller, in some aspects, is adapted to change a location of the zone in response to movement of the tag relative to the patient support apparatus.

The controller, in some aspects, is further adapted to not stop movement of the component if the change in weight is smaller than the first threshold and the change in weight occurs within the selected zone, or if the change in weight is smaller than the second threshold and the change in weight occurs outside the selected zone.

The controller, in some aspects, is adapted to select a first zone if the tag is worn by a patient and to select a second zone different from the first zone if the tag is worn by a caregiver.

In some aspects, the frame defines a footprint and the controller is adapted to use a first value for the first threshold if the tag is positioned inside of the footprint and to use a second value different from the first value for the first threshold if the tag is positioned outside of the footprint.

The controller, in some aspects, is adapted to use a first value for the first threshold if the tag is positioned above a height of the frame and to use a second value different from the first value for the first threshold if the tag is positioned below the height of the frame.

Before the various embodiments disclosed herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to one embodiment of the disclosure;

FIG. 2 is a perspective view of a litter frame of the patient support apparatus;

FIG. 3 is a perspective view of a base of the patient support apparatus;

FIG. 4 is a plan view of a control panel that may be included with the patient support apparatus;

FIG. 5 is a block diagram of a control system of the patient support apparatus, as well as several external devices with which the patient support apparatus may be configured to communicate;

FIG. 6 is a plan view of an illustrative caregiver control panel integrated into an outer face of a siderail of the patient support apparatus;

FIG. 7 is a plan view of an illustrative patient control panel integrated into an inner face of a siderail of the patient support apparatus;

FIG. 8 is a flowchart of an obstruction detection method that may be carried out by the patient support apparatus;

FIG. 9 is an illustrative menu screen displayable in response to a user selecting a menu control on FIG. 4;

FIG. 10 is an illustrative settings screen displayable in response to a user selecting the “settings” option on the screen of FIG. 9;

FIG. 11 is an illustrative motion obstruction sensitivity selection screen displayable in response to a user selecting the “motion obstruction sensitivity” option on the screen of FIG. 10;

FIG. 12 is an illustrative motion obstruction sensitivity selection screen displayable in response to a user taking a secret action enabling the user to disable the motion obstruction function;

FIG. 13 is a plan view graph of the shape and dimensions of a first ingress/egress zone based on a first state of four siderails of the patient support apparatus;

FIG. 14 is a plan view graph of the shape and dimensions of a second ingress/egress zone based on a second state of the four siderails of the patient support apparatus;

FIG. 15 is a plan view graph of the shape and dimensions of a third ingress/egress zone based on a third state of the four siderails of the patient support apparatus;

FIG. 16 is a plan view graph of the shape and dimensions of a fourth ingress/egress zone based on a fourth state of the four siderails of the patient support apparatus;

FIG. 17 is a plan view graph of the shape and dimensions of a fifth ingress/egress zone based on a fifth state of the four siderails of the patient support apparatus;

FIG. 18 is a plan view graph of the shape and dimensions of a sixth ingress/egress zone based on a sixth state of the four siderails of the patient support apparatus;

FIG. 19 is a plan view graph of the shape and dimensions of a seventh ingress/egress zone based on a seventh state of the four siderails of the patient support apparatus;

FIG. 20 is a plan view graph of the shape and dimensions of an eighth ingress/egress zone based on an eighth state of the four siderails of the patient support apparatus;

FIG. 21 is a table defining the four vertices of the ingress/egress zones for all sixteen different states of the four siderails of the patient support apparatus;

FIG. 22 is a graph of several examples of weight changes that may be detected by the scale system of the patient support apparatus, including a weight change due to an obstruction being hit and weight changes due to other causes;

FIG. 23 is an illustrative obstruction detection screen that may be automatically displayed in response to the patient support apparatus hitting an obstruction;

FIG. 24 is the obstruction detection screen of FIG. 23 shown after the user has successfully pressed and held the recovery control of FIG. 23;

FIG. 25 is the obstruction detection screen of FIGS. 23 and 24 shown after the patient support apparatus has been successfully moved out of contact with the obstruction;

FIG. 26 is an illustrative manual override screen that may be displayed in response to a user selecting the information icon on FIG. 23 or 24;

FIG. 27 is the manual override screen of FIG. 26 shown after the user has activated the manual override;

FIG. 28 is a side, elevation view of the patient support apparatus shown positioned next to a wall;

FIG. 29 is an alternative obstruction detection screen that may be displayed in lieu of the screen of FIG. 23 and that indicates that the litter frame needs to be raised in order to move way from an obstruction;

FIG. 30 is another alternative obstruction detection screen that may be displayed in lieu of the screen of FIG. 23 and that indicates that the litter frame needs to be lowered in order to move way from an obstruction;

FIG. 31 is another alternative obstruction detection screen that may be displayed in lieu of the screen of FIG. 23 and that indicates that the Fowler section of the patient support apparatus needs to be lowered in order to move way from an obstruction;

FIG. 32 is another alternative obstruction detection screen that may be displayed in lieu of the screen of FIG. 23 and that indicates that the Fowler section needs to be raised in order to move way from an obstruction;

FIG. 33 is a plan view of a footprint of an embodiment of a patient support apparatus that monitors a dynamic zone defined by an area around a tag worn by a person;

FIG. 34 is a plan view of the footprint of an embodiment of the patient support apparatus that monitors a plurality of predetermined zones, any one or more of which may be selected in response to the presence of a tag worn by a person;

FIG. 35 is a plan view of the footprint of the patient support apparatus of FIG. 34 showing the tag of FIG. 34 moved to a location within two zones; and

FIG. 36 is a plan view of the footprint of an embodiment of the patient support apparatus that includes an exclusion zone.

### DETAILED DESCRIPTION OF THE EMBODIMENTS

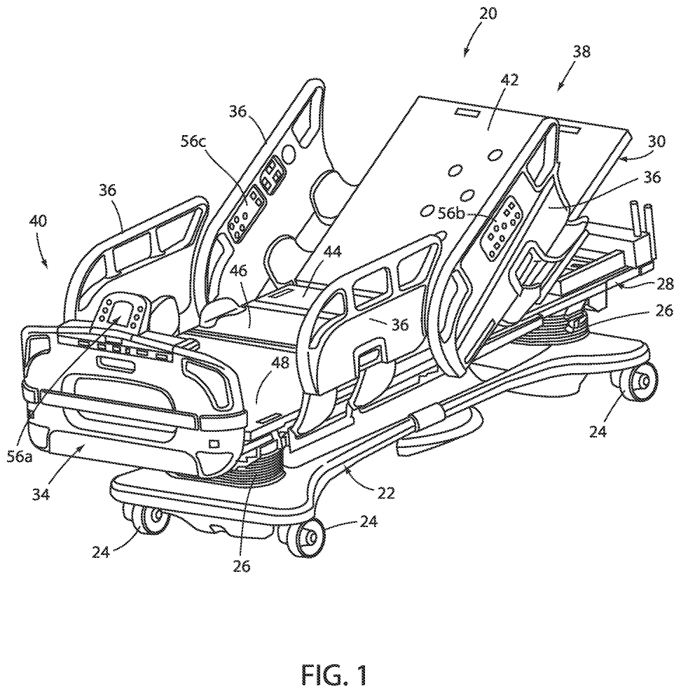

An illustrative patient support apparatus 20 that may incorporate one or more aspects of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a gurney, a recliner, a residential bed, an operating table, or any other structure capable of supporting a patient, whether stationary or mobile and/or whether medical or residential.

In general, patient support apparatus 20 includes a base 22 having a plurality of wheels 24, a pair of lifts 26 supported on the base, a litter frame 28 supported on the lifts 26, and a support deck 30 supported on the litter frame 28. Patient support apparatus 20 further includes a footboard 34, and a plurality of siderails 36. Siderails 36 are all shown in a raised position in FIG. 1 but are each individually movable to a lower position in which ingress into, and egress out of, patient support apparatus 20 is not obstructed by the lowered siderails 36. In some embodiments, siderails 36 may be moved to one or more intermediate positions as well.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, footboard 34, and siderails 36. Support deck 30 provides a support surface for a mattress (not shown), or other soft cushion, so that a person may lie and/or sit thereon. Support deck 30 is made of a plurality of sections, some of which are pivotable about generally horizontal pivot axes. In the embodiment shown in FIG. 1, support deck 30 includes at least a head section 42, a seat section 44, a thigh section 46, and a foot section 48, all of which generally form flat surfaces for supporting the mattress. Head section 42, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

In some embodiments, patient support apparatus 20 may be modified from what is shown to include one or more components adapted to allow the user to extend the width of patient support deck 30, thereby allowing patient support apparatus 20 to accommodate patients of varying sizes. When so modified, the width of deck 30 may be adjusted sideways in any increments, for example between a first or minimum width, a second or intermediate width, and a third or expanded/maximum width. Notionally, the first standard width may be considered a 36 inch width, the second intermediate width may be considered a 42 inch width and the third more expanded width may be considered a 48 inch width, although these numerical widths may be varied to comprise different width values.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions, such as, but not limited to, that described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may be the same as, or nearly the same as, the mechanical construction of the Model 3002 S3 bed manufactured and sold by Stryker Corporation of Kalamazoo, Michigan. This mechanical construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. It will be understood by those skilled in the art that patient support apparatus 20 can be designed with still other types of mechanical constructions, such as, but not limited to, those described in commonly assigned, U.S. Pat. No. 7,690,059 issued to Lemire et al., and entitled HOSPITAL BED; and/or commonly assigned U.S. Pat. publication No. 2007/0163045 filed by Becker et al, and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The mechanical construction of patient support apparatus 20 may also take on still other forms different from what is disclosed in the aforementioned references.

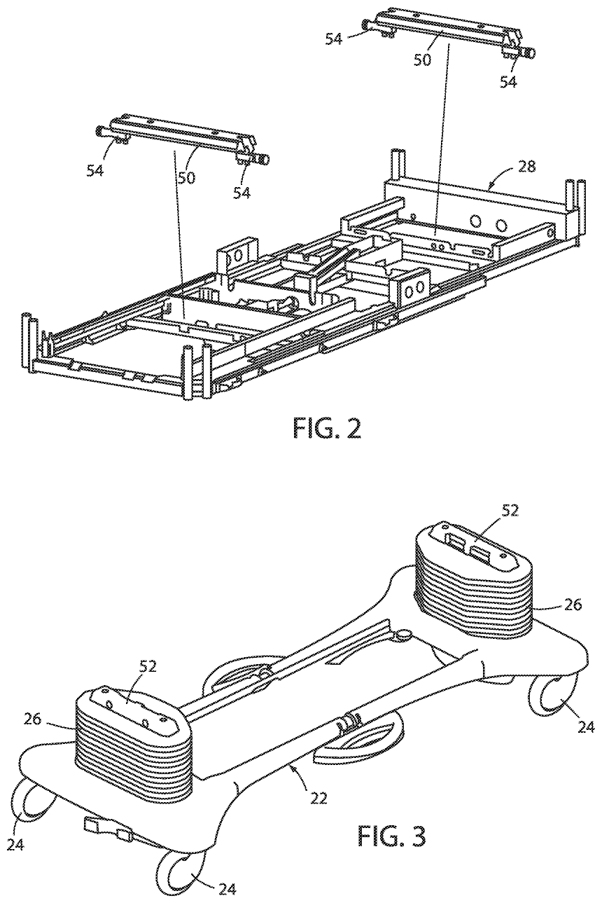

FIG. 2 illustrates in greater detail litter frame 28 separated from lifts 26 and base 22. Litter frame 28 is also shown in FIG. 2 with support deck 30 removed. Litter frame 28 is supported by two lift header assemblies 50. A first one of the lift header assemblies 50 is coupled to a top 52 (FIG. 3) of a first one of the lifts 26, and a second one of the lift header assemblies 50 is coupled to the top 52 of the second one of the lifts 26. Each lift header assembly 50 includes a pair of force sensors 54, which may be load cells, or other types of force sensors. The illustrated embodiment of patient support apparatus 20 therefore includes a total of four force sensors 54, although it will be understood by those skilled in the art that different numbers of force sensors may be used in accordance with the principles of the present disclosure. Force sensors 54 are configured to support litter frame 28. More specifically, force sensors 54 are configured such that they provide complete and exclusive mechanical support for litter frame 28 and all of the components that are supported on litter frame 28 (e.g. support deck 30, footboard 34, the headboard, siderails 36, etc.). Because of this construction, force sensors 54 are adapted to detect the weight of not only those components of patient support apparatus 20 that are supported by litter frame 28 (including litter frame 28 itself), but also any objects or persons who are wholly or partially being supported by support deck 30. The outputs of force sensors 54 are used for implementing an obstruction detection system and, in some embodiments, for also implementing a scale function and/or an exit detection function, as will be described in greater detail below.

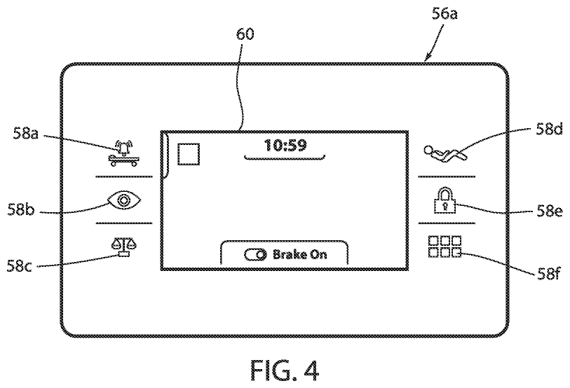

Patient support apparatus 20 further includes a plurality of control panels 56 (FIG. 1) that enable a user of patient support apparatus 20, such as a patient and/or an associated caregiver, to control one or more aspects of patient support apparatus 20. In the embodiment shown in FIG. 1, patient support apparatus 20 includes a footboard control panel 56a, a pair of outer siderail control panels 56b (only one of which is visible), and a pair of inner siderail control panels 56c (only one of which is visible). Footboard control panel 56a and outer siderail control panels 56b are intended to be used by caregivers, or other authorized personnel, while inner siderail control panels 56c are intended to be used by the patient associated with patient support apparatus 20. Each of the control panels 56 includes a plurality of controls 58 (see, e.g. FIGS. 4-7), although each control panel 56 does not necessarily include the same controls and/or functionality.

Among other functions, controls 58 of control panel 56a allow a user to control one or more of the following: change a height of support deck 30, raise or lower head section 42, take patient weight readings, arm and disarm an exit detection function, control an obstruction detection system, activate and deactivate a brake for wheels 24, arm and disarm one or more patient support apparatus monitoring functions, change various settings on patient support apparatus 20, view the current location of the patient support apparatus 20 as determined by a location detection system, perform a calibration process on the patient support apparatus 20, and perform still other actions. One or both of the inner siderail control panels 56c also include at least one control 58 that enables a patient to call a remotely located nurse (or other caregiver).

Control panel 56a includes a display 60 (FIG. 4) configured to display a plurality of different screens thereon. Surrounding display 60 are a plurality of navigation controls 58a-f that, when activated, cause the display 60 to display different screens on display 60. For example, when a user presses navigation control 58a, control panel 56a displays an exit detection control screen on display 60 that includes one or more icons that, when touched, control an onboard exit detection function. The exit detection function is adapted to issue an alert when a patient exits from patient support apparatus 20. Such an exit detection function may include any of the same features and/or functions as, and/or may be constructed in any of the same manners as, the exit detection systems disclosed in commonly assigned U.S. patent application 62/889,254 filed Aug. 20, 2019, by inventors Sujay Sukumaran et al, and entitled PERSON SUPPORT APPARATUS WITH ADJUSTABLE EXIT DETECTION ZONES, and/or the exit detection system disclosed in commonly assigned U.S. Pat. No. 5,276,432 issued to Travis and entitled PATIENT EXIT DETECTION MECHANISM FOR HOSPITAL BED, the complete disclosures of both of which are incorporated herein by reference.

When a user presses navigation control 58b (FIG. 4), control panel 56a displays a monitoring control screen that includes a plurality of control icons that, when touched, control an onboard monitoring system that may be built into patient support apparatus 20. The onboard monitoring system alerts the caregiver through a unified indicator, such as a light or a plurality of lights controlled in a unified manner, when any one or more of a plurality of settings on patient support apparatus 20 are in an undesired state, and uses that same unified indicator to indicate when all of the plurality of settings are in their respective desired states. Stated alternatively, the monitoring system, when armed, monitors a plurality of conditions of patient support apparatus 20 (such as, but not limited to, any one or more of the following: the brake status, the siderail positions, the litter frame height, the exit detection system, the obstruction detection system, the A/C cord status, the nurse call cable status, etc.) and issues an alert if any one of those conditions are in an undesired state. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al, and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al, and entitled PATIENT SUPPORT APPARATUSES WITH MOTION CUSTOMIZATION, the complete disclosures of both of which are incorporated herein by reference. Other types of monitoring systems may be included within patient support apparatus 20.

When a user presses navigation control 58c, control panel 56a displays a scale control screen that includes a plurality of control icons that, when touched, control the scale system of patient support apparatus 20. When included, the scale system of patient support apparatus 20 may include any of the same features, components, and/or and functions as the scale systems disclosed in commonly assigned U.S. patent application 62/889,254 filed Aug. 20, 2019, by inventors Sujay Sukumaran et al, and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al, and entitled PATIENT SUPPORT APPARATUS WITH EQUIPMENT WEIGHT LOG, the complete disclosures of both of which are incorporated herein by reference. The scale system may utilize the same force sensors that are utilized by the exit detection system and/or the obstruction detection system, in some embodiments, or it may utilize one or more different sensors.

When a user presses navigation control 58d, control panel 56 displays a motion control screen that includes a plurality of control icons that, when touched, control the movement of various components of patient support apparatus 20, such as, but not limited to, the height of litter frame 28 and the pivoting of head section 42. In some embodiments, the motion control screen displayed on display 60 in response to pressing control 58d may be the same as, or similar to, the position control screen 216 disclosed in commonly assigned U.S. patent application Ser. No. 62/885,953 filed Aug. 13, 2019, by inventors Kurosh Nahavandi et al, and entitled PATIENT SUPPORT APPARATUS WITH TOUCHSCREEN, the complete disclosure of which is incorporated herein by reference. Other types of motion control screens may be included on patient support apparatus 20.

When a user presses navigation control 58e (FIG. 4), control panel 56a displays a motion lock control screen that includes a plurality of control icons that, when touched, control one or more motion lockout functions of patient support apparatus 20. Such a motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al, and entitled PATIENT SUPPORT APPARATUSES WITH MOTION CUSTOMIZATION, the complete disclosure of which is incorporated herein by reference. Other types of motion lockouts may be included within patient support apparatus 20.

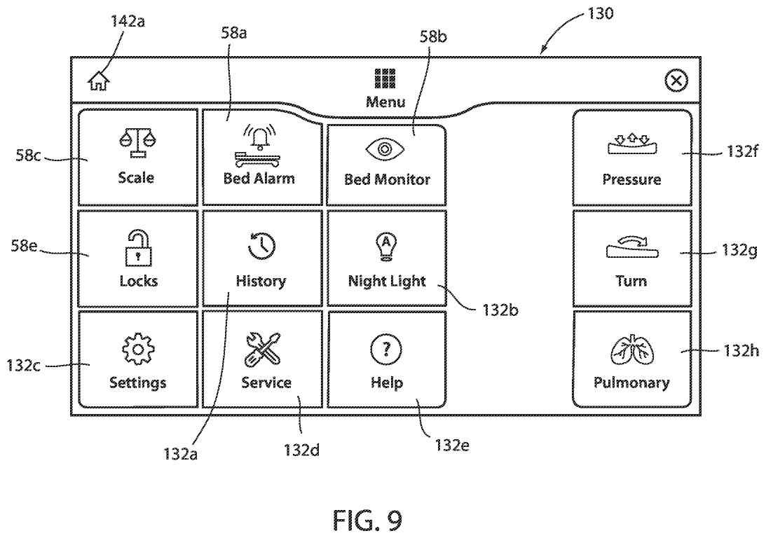

When a user presses on navigation control 58f, control panel 56a displays a menu screen (e.g. menu screen 130 of FIG. 9) that includes a plurality of menu icons that, when touched, bring up one or more additional screens for controlling and/or viewing one or more other aspects of patient support apparatus 20. Such other aspects include, but are not limited to, settings related to the obstruction detection system, diagnostic and/or service information for patient support apparatus 20, mattress control and/or status information, configuration settings, location information, medical device association information, and other settings and/or information. One example of a menu screen in shown herein in FIG. 9. Another example of a suitable menu screen is the menu screen 100 disclosed in commonly assigned U.S. patent application Ser. No. 62/885,953 filed Aug. 13, 2019, by inventors Kurosh Nahavandi et al, and entitled PATIENT SUPPORT APPARATUS WITH TOUCHSCREEN, the complete disclosure of which is incorporated herein by reference. Other types of menus and/or settings may be included within patient support apparatus 20.

For all of the navigation controls 58a-f (FIG. 4), screens other than the ones specifically mentioned above may be displayed on display 60 in other embodiments of patient support apparatus 20 in response to a user pressing these controls. Thus, it will be understood that the specific screens mentioned above are merely representative of the types of screens that are displayable on display 60 in response to a user pressing on one or more of navigation controls 58a-f. It will also be understood that, although navigation controls 58a-f have all been illustrated in the accompanying drawings as dedicated controls that are positioned adjacent display 60, any one or more of these controls 58a-f could alternatively be touchscreen controls that are displayed at one or more locations on display 60. Still further, although controls 58a-f have been shown herein as buttons, it will be understood that any of controls 58a-f could also, or alternatively, be switches, dials, or other types of non-button controls.

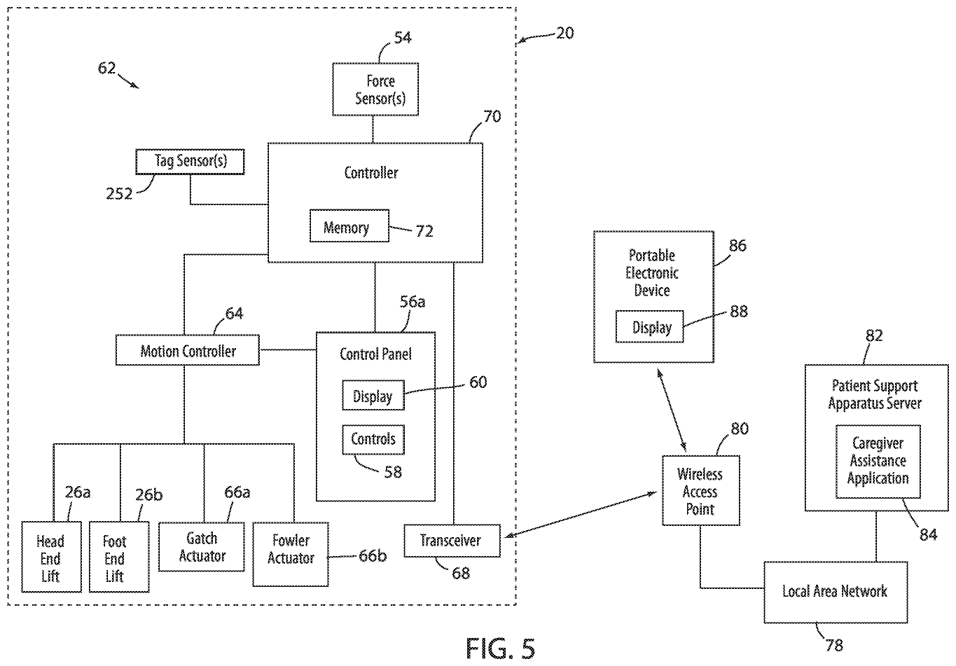

As shown in FIG. 5, patient support apparatus 20 includes a control system 62 that controls various aspects of patient support apparatus 20. Control system 62 includes control panels 56 (only one of which—56a—is shown in FIG. 5), force sensors 54, a motion controller 64, a plurality of motorized actuators 26 and 66, a network transceiver 68, and a main controller 70. Main controller 70 includes a memory 72. Main controller 70 and force sensors 54 may act together to perform both a scale function and an exit detection function, i.e. they may comprise a scale system and/or an exit detection system. Main controller 70 and force sensors 54 may also, or alternatively, act together to implement an obstruction detection system that implements an obstruction detection function. When carrying out the exit detection function, main controller 70 and force sensors 54 are adapted to determine when an occupant leaves patient support apparatus 20, or moves in a way indicative of an imminent departure from the patient support apparatus. In such situations, main controller 70 is configured to issue an alert and/or notification to appropriate personnel so that proper steps can be taken in response to the occupant's departure, or imminent departure, in a timely fashion. When carrying out the scale function, the outputs of force sensors 54 are summed together to determine a patient's weight (after adjusting for the tare weight and/or other weights that may be stored in an equipment weight log). When carrying out the obstruction detection function, the outputs of force sensors 54 are analyzed while one or more actuators 26 and/or 66 are being driven and the analysis is used to determine if an obstruction is encountered during the movement of one or more components of patient support apparatus 20. Further details of the various features of the obstruction detection system are described in detail below.

Force sensors 54 are adapted to detect downward forces exerted by an occupant of support deck 30. Thus, when an occupant is positioned on support deck 30 and remains substantially still (i.e. not moving in a manner involving accelerations that cause forces to be exerted against support deck 30), force sensors 54 will detect the weight of the occupant (as well as the weight of any components of patient support apparatus 20 that are supported—directly or indirectly—by force sensors 54). In at least one embodiment, force sensors 54 are load cells. However, it will be understood by those skilled in the art, that force sensors 54 may be implemented as other types of sensors, such as, but not limited to, linear variable displacement transducers and/or any one or more capacitive, inductive, and/or resistive transducers that are configured to produce a changing output in response to changes in the force exerted against them.

Main controller 70 and motion controller 64 are constructed of any electrical component, or group of electrical components, that are capable of carrying out the functions described herein. In many embodiments, controllers 64 and 70 are conventional microcontrollers, although not all such embodiments need include a microcontroller. In general, controllers 64 and 70 include any one or more microprocessors, microcontrollers, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. Indeed, in some embodiments, main controller 70 and motion controller 64 are combined with each other and/or with other circuitry or other controllers that are present on patient support apparatus 20. The instructions followed by controllers 64 and 70 in carrying out the functions described herein, as well as the data necessary for carrying out these functions, are stored in one or more memories that are accessible to them (e.g. memory 72 for main controller 70).

Although patient support apparatus 20 includes a total of four force sensors 54, it will be understood by those skilled in the art that different numbers of force sensors 54 may be used in accordance with the principles of the present disclosure. Force sensors 54, in at least one embodiment, are configured to support litter frame 28. When so configured, force sensors 54 are constructed to provide complete and exclusive mechanical support for litter frame 28 and all of the components that are supported on litter frame 28 (e.g. deck 30, footboard 34, and, in some embodiments, siderails 36). Because of this construction, force sensors 54 are adapted to detect the weight of not only those components of patient support apparatus 20 that are supported by the litter frame 28 (including litter frame 28 itself), but also any objects or persons who are positioned either wholly or partially on support deck 30. By knowing the weight of the components of the patient support apparatus 20 that are supported on litter frame 28, controller 70 is able to determine a tare weight that, when subtracted from a total weight sensed after a patient is supported on support deck 30, yields a patient weight.

In some embodiments, the physical location of the force sensors 54 on patient support apparatus 20 may be modified to be located on the base frame, such as shown in commonly assigned U.S. patent application Ser. No. 62/889,254 filed Aug. 20, 2019, by inventors Sujay Sukumaran et al, and entitled PERSON SUPPORT APPARATUS WITH ADJUSTABLE EXIT DETECTION ZONES, the complete disclosure of which is incorporated herein by reference. In other embodiments, the physical location of the force sensors 54 on patient support apparatus 20 may be the same as the position of the load cells disclosed in commonly assigned U.S. patent application Ser. No. 15/266,575 filed Sep. 15, 2016, by inventors Anuj Sidhu et al, and entitled PERSON SUPPORT APPARATUSES WITH EXIT DETECTION SYSTEMS, the complete disclosure of which is also incorporated herein by reference. In still other embodiments, the physical location of the force sensors 54 may be the same as the position of the load cells disclosed in U.S. Pat. No. 7,962,981 issued to Lemire et al, and entitled HOSPITAL BED, the complete disclosure of which is also incorporated herein by reference. In still other embodiments, force sensors 54 may be positioned on patient support apparatus 20 at still other locations.

Motion controller 64 (FIG. 5) is adapted to control the movement of a plurality of components of patient support apparatus 20. These components includes, but are not limited to, a head end lift actuator 26a, a foot end lift actuator 26b, a gatch actuator 66a, and a Fowler actuator 66b. Each of these actuators 26a-b, 66a-b may comprise a linear actuator with a motor built therein. In some embodiments, the linear actuator may be of the type disclosed in commonly assigned U.S. patent application Ser. No. 15/449,277 filed Mar. 3, 2017, by inventors Anish Paul et al, and entitled PATIENT SUPPORT APPARATUS WITH ACTUATOR FEEDBACK, the complete disclosure of which is incorporated herein by reference. In other embodiments, other types of powered actuators may be used, such as, but not limited to, hydraulic and/or pneumatic actuators.

In some embodiments, motion controller 64 operates in the same or similar manners to the main microcontroller 58 and its associated circuitry disclosed in commonly assigned U.S. Pat. No. 10,420,687 issued Sep. 24, 2019, to inventors Aaron Furman et al, and entitled BATTERY MANAGEMENT FOR PATIENT SUPPORT APPARATUSES, the complete disclosure of which is incorporated herein by reference. In such embodiments, motion controller 64 controls the sending of pulse width modulated (PWM) signals to the motors contained within actuators 26a,b and/or 66a, b, thereby controlling both the speed and the direction of movement of these actuators. Motion controller 64 may take on other forms as well.

Motion controller 64 is in communication with control panel 56 and receives signals from control panel 56 indicating when a user wishes to move one or more components of patient support apparatus 20. That is, control panel 56 includes one or more controls 58 that are adapted, when activated, to instruct motion controller 64 to drive one or more of the actuators 26, 66 in order to carry out the desired movement of the various movable components of patient support apparatus 20. In some embodiments, when the user stops pressing, or otherwise activating, the movement controls 58, motion controller 64 automatically stops driving the one or more actuators 26, 66 and movement of the one or more components automatically stops. Further, if an obstruction is encountered, main controller 70 and/or motion controller 64 are adapted to automatically stop driving the actuators 26, 66 so that continued movement of the one or more components towards the obstacle stops. The movements controllable by control panels 56 includes, but is not limited to, raising and lowering the height of litter frame 28 (and/or the individual ends 38 and 40 of litter frame 28), pivoting the Fowler section 42 up and down about a generally horizontal axis (extending laterally from one side of the patient support apparatus 20 to the other), lifting and lowering a knee gatch on patient support apparatus 20, and/or combinations of these movements.

Head end lift actuator 26a is configured to change the height of the head end 38 of litter frame 28. Foot end lift actuator 26b is configured to change the height of the foot end 40 of litter frame 28. When both of these actuators 26a and 26b are operated simultaneously and at the same speed, the height of litter frame 28 is raised or lowered without changing the general orientation of litter frame 28 with respect horizontal. When one or more of these actuators 26a and/or 26b are operated at different times and/or at different speeds, the orientation of litter frame 28 is changed with respect to horizontal. Lift actuators 26a and 26b are therefore able to tilt litter frame 28 to a variety of different orientations, including, but not limited to, a Trendelenburg orientation, a reverse-Trendelenburg orientation, a vascular orientation, an egress orientation, and/or other orientations.

Gatch actuator 66a is adapted to raise and lower the joint that couples together the thigh section 46 and the foot section 48 of support deck 30, thereby raising and lowering the portion of the support deck 30 that is positioned close to the patient's knees. Fowler actuator 66b is adapted to raise and lower the head section (or Fowler section) 42 of the support deck 30.

Control panel 56 (FIG. 4) communicates with main controller 70 and is adapted to allow a user to control the various functions performed by main controller 70, including, but not limited to, the obstruction detection function the scale function, and the exit detection function carried out by main controller 70 (and force sensors 54). The plurality of controls 58 of control panel 56 may be implemented as buttons, dials, switches, icons on a touchscreen, or other devices. Display 60 of control panel 56 may be a touchscreen that displays one or more controls and/or one or more control screens, some of which are discussed in greater detail below. Display 60 may comprise an LED display, OLED display, or another type of display.

Control system 62 may include additional components beyond those shown in FIG. 5. Alternatively, or additionally, one or more of the components shown in FIG. 5 may be omitted from patient support apparatus 20, or replaced by other components. In some embodiments, control system 62 is configured to automatically maintain a weight log of non-patient items added to, and removed from, the patient support apparatus, thereby making it easier for the caregiver to take accurate readings of the patient's weight. In some such embodiments, control system 62 includes any of the additional components and/or functionality of the scale systems disclosed in the following commonly assigned U.S. patent references: U.S. patent application Ser. No. 63/342,899 filed May 17, 2022, by inventors Sujay Sukumaran et al, and entitled PATIENT SUPPORT APPARATUS WITH AUTOMATIC SCALE FUNCTIONALITY; U.S. Pat. No. 10,357,185 issued to Marko Kostic et al. on Jul. 23, 2019, and entitled PERSON SUPPORT APPARATUSES WITH MOTION MONITORING; U.S. Pat. No. 11,033,233 issued to Michael Hayes et al. on Jun. 15, 2021, and entitled PATIENT SUPPORT APPARATUS WITH PATIENT INFORMATION SENSORS; and U.S. patent application Ser. No. 16/992,515 filed Aug. 13, 2020, by inventors Kurosh Nahavandi et al, and entitled PATIENT SUPPORT APPARATUS WITH EQUIPMENT WEIGHT LOG, the complete disclosures of all of which are incorporated herein by reference.

Network transceiver 68 (FIG. 5) is adapted to communicate with a hospital's local area network 78. In some embodiments, network transceiver 68 is a conventional WiFi transceiver (i.e. IEEE 802.11 . . . ) adapted to wirelessly communicate with one or more wireless access points 80 of a hospital's local area network 78. In other embodiments, network transceiver 68 may be a wireless transceiver that uses conventional 5G technology to communicate with network 78, one or more servers hosted thereon, and/or other devices. In some embodiments, network transceiver 68 may include any of the structures and/or functionality of the communication modules 56 disclosed in commonly assigned U.S. Pat. No. 10,500,401 issued to Michael Hayes and entitled NETWORK COMMUNICATION FOR PATIENT SUPPORT APPARATUSES, the complete disclosure of which is incorporated herein by reference. Still other types of wireless network transceivers may be utilized.

In other embodiments, network transceiver 68 may be a conventional Ethernet transceiver electrically coupled to a conventional Ethernet port (i.e. RJ-45 jack, or the like) built into patient support apparatus 20 that allows a conventional Ethernet cable to be coupled to the patient support apparatus 20. In these embodiments, patient support apparatuses 20 may be coupled to the hospital's local area network 78 by a wired connection. In still other embodiments, patient support apparatus 20 may have both wired and wireless transceivers 68. Still further, in some embodiments, transceiver 68 may take on yet a different form (e.g. a wireless ZigBee transceiver, a Bluetooth transceiver, etc.).

Patient support apparatus 20 uses transceiver 68 (FIG. 5), in some embodiments, to communicate with a patient support apparatus server 82. Patient support apparatus server 82 may be adapted to receive status information from patient support apparatuses 20 and distribute that information to one or more other servers and/or other devices coupled to local area network 78. In at least one embodiment, patient support apparatus server 82 includes a caregiver assistance software application 84 that is adapted to communicate information between both patient support apparatuses 20 and one or more portable electronic devices 86. The portable electronic devices 86 includes, but are not limited to, smart phones, tablets, laptops, Computers on Wheels (COWs), and the like. Each portable electronic device 86 includes a display 88 on which various screens may be displayed, including, in some embodiments, portions of one or more of the screens discussed below. In some embodiments, caregiver assistance application 84 allows authorized users to remotely configure and remotely control various aspects of the patient support apparatuses 20 using their portable electronic device 86, such as, but not limited to, the obstruction detection system. Still further, caregiver assistance application 84 may be adapted to display information about the obstruction detection system, scale system, exit detection system, and/or other information of the patient support apparatuses 20, including any of the information discussed in greater detail below regarding the obstruction detection system.

In any of the embodiments disclosed herein, caregiver assistance application 84 may be configured to include any of the same features or functions as—and/or to operate in any of the same manners as—the caregiver assistance software applications described in the following commonly assigned patent applications: U.S. patent application Ser. No. 62/826,097, filed Mar. 29, 2019 by inventors Thomas Durlach et al, and entitled PATIENT CARE SYSTEM; U.S. patent application Ser. No. 16/832,760 filed Mar. 27, 2020, by inventors Thomas Durlach et al, and entitled PATIENT CARE SYSTEM; and/or PCT patent application serial number PCT/US2020/039587 filed Jun. 25, 2020, by inventors Thomas Durlach et al, and entitled CAREGIVER ASSISTANCE SYSTEM, the complete disclosures of which are all incorporated herein by reference. Thus, server 82 may be configured to share with one or more electronic devices 86 any of the information shared with the electronic devices disclosed in these aforementioned patent applications. For example, server 82 may be configured to not only share the location of patient support apparatuses 20 (and any devices that may be associated with them) with electronic devices 86, but it may also forward any of the data generated by patient support apparatuses 20 to the electronic devices 86, thereby letting the caregivers associated with these patient support apparatuses 20 know if, for example, the patient has exited patient support apparatus 20, what the patient's current weight is, whether the patient support apparatus has encountered an obstruction during movement, etc. Alternatively, or additionally, patient support apparatus server 82 may forward other patient support apparatus status data (e.g. current siderail position, bed exit status, brake status, height status, scale data, etc.) and/or caregiver rounding information (e.g. when the last rounding was performed for a particular patient, when the next rounds are due, etc.), and/or object data from any objects supported on patient support apparatus 20 to one or more electronic devices 86, thereby providing the caregivers associated with the devices 86 a consolidated portal (e.g. a single software application) for sharing this various information.

It will be understood that, although caregiver assistance application 84 is shown in FIG. 5 to be executed solely by server 82, this may be modified so that caregiver assistance application 84 is executed, either partially or wholly, on one or more of the mobile devices 86 as a mobile app that is downloaded to these devices 86. Similarly, caregiver assistance application 84 may be implemented as an app that executes on server 82 in conjunction with another specialized app that is downloaded to each of the mobile electronic devices 86. Still other variations of caregiver assistance application 84 are possible.

In some embodiments, patient support apparatus server 82 is also configured to determine the location of each patient support apparatus 20, or receive the location of each patient support apparatus 20 from the patient support apparatuses 20. In some embodiments, patient support apparatus server 82 determines the room number and/or bay area of each patient support apparatus 20 that is positioned within a room, as well as the location of patient support apparatuses 20 that are positioned outside of a room, such as, those that may be positioned in a hallway, a maintenance area, or some other area. In general, patient support apparatus server 82 may be configured to determine the position of any patient support apparatus 20 by communicating with one or more nearby wall units (not shown). Further details regarding several manners in which patient support apparatus 20 may be constructed in order to carry out such location communication, as well as the construction and/or operation of such wall units, are disclosed in the following commonly assigned U.S. patent applications: Ser. No. 63/245,245 filed Sep. 17, 2021, by inventors Kirby Niehouser et al., and entitled SYSTEM FOR LOCATING PATIENT SUPPORT APPARATUSES; Ser. No. 63/245,289 filed Sep. 17, 2021, by inventors Madhu Sandeep Thota et al, and entitled PATIENT SUPPORT APPARATUS COMMUNICATION AND LOCATION SYSTEM; Ser. No. 63/026,937 filed May 19, 2020, by inventors Alexander Bodurka et al, and entitled PATIENT SUPPORT APPARATUSES WITH HEADWALL COMMUNICATION; and Ser. No. 63/193,778 filed May 27, 2021, by inventors Krishna Bhimavarapu et al, and entitled PATIENT SUPPORT APPARATUS AND HEADWALL UNIT SYNCHING; the complete disclosures of all of which have already been incorporated herein by reference.

It will be understood that the architecture and content of local area network 78 will vary from healthcare facility to healthcare facility, and that FIG. 5 merely shows a generic example of the type of network a healthcare facility may be employ. Typically, one or more additional servers will be hosted on network 78 and one or more of them may be adapted to communicate with patient support apparatus server 82. For example, an electronic health record server will typically be present in any healthcare facility, and in some embodiments discussed herein, it will be in communication with patient support apparatus server 82 in order to receive patient data that is to be recorded in a patient's health record (e.g. weight readings taken from the scales built into patient support apparatuses 20; therapies provided to patients using a powered mattress onboard patient support apparatuses 20, etc.).

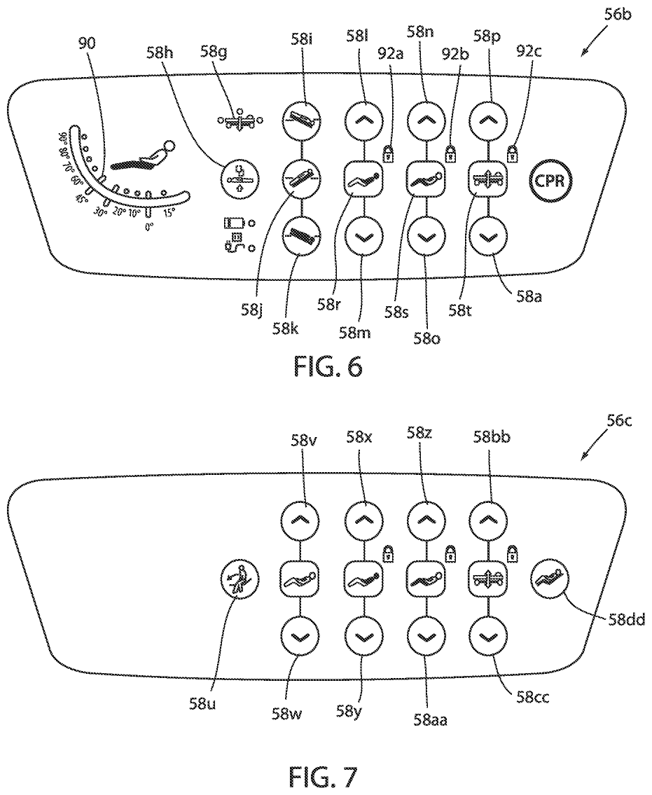

Turning to FIG. 6, an illustrative caregiver control panel 56b comprising a plurality of controls 58 is shown. Caregiver control panel 56b is, in at least one embodiment, positioned on an outside face of one of the siderails 36. In the embodiment shown in FIG. 1, patient support apparatus 20 includes two caregiver control panels 56b (as well as two patient control panels 56c). Caregiver control panel 56b includes an angle indicator 90, a flat configuration control 58g, an examination position control 58h, a Trendelenburg control 58i, a reverse Trendelenburg control 58j, a vascular position control 58k, a knee up control 58l, a knee down control 58m, a Fowler up control 58n, a Fowler down control 580, a height up control 58p, a height down control 58q, a knee manual lockout control 58r, a Fowler manual lockout control 58s, and a manual height lockout control 58t. Caregiver control panel 56b further includes a knee lockout indicator 92a, a Fowler lockout indicator 92b, and a height lockout indicator 92c.

Each caregiver control panel 56b is in electrical communication with controller 70. When a caregiver presses on examination control 58h, controller 70 is configured to automatically control the movement of one or more actuators 26 and/or 66 to bring frame 28 and support deck 30 to a predefined examination position. Although the predefined positions of the litter frame 28 and support deck 30 may vary for a given examination position, in at least one embodiment, controller 70 is configured to move the support deck 30 and litter frame 28 to flat orientations, and to raise the height of the litter frame 28 to its maximum extent (or to a different height that makes it easy for a caregiver to examine the patient while he/she is positioned on patient support apparatus 20).

When a caregiver presses on Trendelenburg control 58i, controller 70 is configured to automatically control the movement of lifts 26 so as to bring litter frame 28 to a conventional Trendelenburg position. In the Trendelenburg position, the litter frame 28 is tilted so that head end 38 is positioned lower than foot end 40. The Fowler section and/or knee section of support deck 30 may be flat or raised while in the Trendelenburg position. When a caregiver presses on reverse Trendelenburg control 58j, controller 70 is configured to automatically control the movement of lifts 26 so as to bring litter frame 28 to a conventional reverse Trendelenburg position. In the reverse Trendelenburg position, the litter frame 28 is tilted so that foot end 40 is positioned lower than head end 38. The Fowler section and/or knee section of support deck 30 may be flat or raised while in the reverse Trendelenburg position. When a caregiver presses on vascular control 58k, controller 70 is configured to automatically control the movement of lifts 26 and actuators 66 so as to bring litter frame 28 and deck 30 to a vascular position. In the vascular position, the Fowler section and knee section of patient support deck 30 are lowered to a flat orientation (zero degree angle with the main plane of the support deck 30) and lifts 26 are controlled so as to lower head end 38 lower than foot end 40. The vascular position is similar to the Trendelenburg position except that it also flattens the Fowler section and knee sections. The angle of litter frame 28 when in the vascular position may be the same as, or different from, the angle of litter frame 28 when it is in the Trendelenburg position.

When a caregiver presses on knee up control 58l or knee down control 58m, controller 70 is configured to raise or lower, respectively, the knee section of support deck 30 by activating knee actuator 66a. When a caregiver presses on Fowler up control 58n or Fowler down control 580, controller 70 is configured to raise or lower, respectively, the Fowler section or support deck 30 by activating Fowler actuator 66b. When a caregiver presses on height up control 58p or height down control 58q, controller 70 is configured to raise or lower, respectively, the height of litter frame 28 by activating lifts 26.

Lockout indicators 92a-c (FIG. 6) are illuminated when a corresponding control 58 has been locked out. For example, knee lockout indicator 92a is illuminated when the knee up or knee down controls 58x and 50y (shown in FIG. 7 and discussed more below) are locked out. Controls 58x and 50y are manually locked out when a caregiver presses on knee lockout control 58r. In some embodiments, knee lockout control 58r is a toggle switch such that repeated pressing of control 58r causes controller 70 to alternatingly lock and unlock controls 58x and 50y. When knee lockout control 58r is activated, controls 58x and 50y are locked out such that if a patient presses on knee up or knee down controls 58x or 50y, controller 70 does not activate knee actuator 66a and therefore does not raise or lower the knee section of patient support apparatus 20.

Fowler lockout indicator 92b (FIG. 6) is illuminated when Fowler controls 58z and 50aa on patient control panel 56c (FIG. 7) are locked out. Controls 58z and 50aa are manually locked out when a caregiver presses on Fowler lockout control 58s on caregiver control panel 56b (FIG. 6). As with knee lockout control 58r, Fowler lockout control 58s may be a toggle switch such that repeated pressing of control 58s causes controller 70 to alternatingly lock and unlock controls 58z and 50aa. When controls 58z and 50aa are locked out, if a patient presses on Fowler up or Fowler down 50z or 50aa, controller 70 does not activate Fowler actuator 66b and therefore does not raise or lower the Fowler section of support deck 30.

Height lockout indicator 92c (FIG. 6) is illuminated when height controls 58bb and 50cc on patient control panel 56c (FIG. 7) are locked out. Controls 58bb and 50cc are manually locked out when a caregiver presses on height lockout control 58t on caregiver control panel 56b (FIG. 6). As with the other manual lockout controls 58r and 50s, height lockout control 58t may be a toggle switch such that repeated pressing of control 58t causes controller 70 to alternatingly lock and unlock controls 58bb and 50cc. When controls 58bb and 50cc are locked out, if a patient presses on height up or height down control 58bb or 50aa, controller 70 does not activate lifts 26 and therefor does not raise or lower litter frame 28.

It will be understood that the locking out of a control serves to disable that control on the patient's control panel 56c. The locking out of a control does not disable the corresponding control on caregiver control panel 56a or 56b, in at least some embodiments. In other embodiments, controller 70 may be configured to lock out a control 58 both on the patient control panels 56c and the two siderail caregiver control panels 56b, but not lock out the corresponding control on footboard control panel 56a. Other arrangements of what controls are locked out on what control panels may also, or alternatively, be implemented.

Turning to FIG. 7, an illustrative patient control panel 56c comprising a plurality of controls 58 is shown. Patient control panel 56c is, in at least one embodiment, positioned on an inside face of one of the siderails 36. In the embodiment shown in FIG. 1, patient support apparatus 20 includes two patient control panels 56c. Patient control panel 56c includes an exit control 58u, a combined Fowler and knee up control 58v, a combined Fowler and knee down control 58w, a knee up control 58x, a knee down control 58y, a Fowler up control 58z, a Fowler down control 58aa, a height up control 58bb, a height down control 58cc, and a chair control 58dd.

When a patient presses on exit control 58u, controller 70 is configured to control the operation of lifts 26, knee actuator 66a, and Fowler actuator 66b to move litter frame 28 and support deck 30 to an overall configuration that makes it easier for a patient to get in or out of patient support apparatus 20. In general, this exit configuration has the Fowler section raised, the knee section flat, and the overall height of litter frame 28 at a height that is conducive for the patient to exit or enter patient support apparatus 20. When a patient presses on combined controls 58v or 50w, controller 70 is configured to raise, or lower, both the knee section and the Fowler section together. That is, if the patient presses on up control 58v, controller 70 is configured to activate both Fowler actuator 66b and knee actuator 66a such that both the Fowler section and the knee section are raised. If the patient presses on down control 58w, controller 70 is configured to activate both Fowler actuator 66b and knee actuator 66a such that both the Fowler section and the knee section are lowered.

Controls 58x, 50y, 50z, 50aa, 50bb, and 50cc of patient control panel 56c (FIG. 7) are the same as caregiver controls 58l, 50m, 50n, 500, 50p, and 50q, respectively, of caregiver control panel 56b (FIG. 6). That is, when a patient presses on knee up control 58x or knee down control 58y, controller 70 is configured to raise or lower, respectively, the knee section of support deck 30 by activating knee actuator 66a. When a patient presses on Fowler up control 58z or Fowler down control 58aa, controller 70 is configured to raise or lower, respectively, the Fowler section or support deck 30 by activating Fowler actuator 66b. When a patient presses on height up control 58bb or height down control 58cc, controller 70 is configured to raise or lower, respectively, the height of litter frame 28 by activating lifts 26.

When a patient presses on chair control 58dd, controller 70 is configured to control lifts 26, Fowler actuator 66b, and knee actuator 66a such that the overall configuration of support deck 30 and litter frame 28 is moved to positions and orientations that resemble a chair. In general, this involves pivoting the Fowler section to a raised orientation, lowering a foot section of support deck 30 to a lowered position and, in some embodiments, tilting litter frame 28 so that head end 38 is positioned higher than foot end 40.

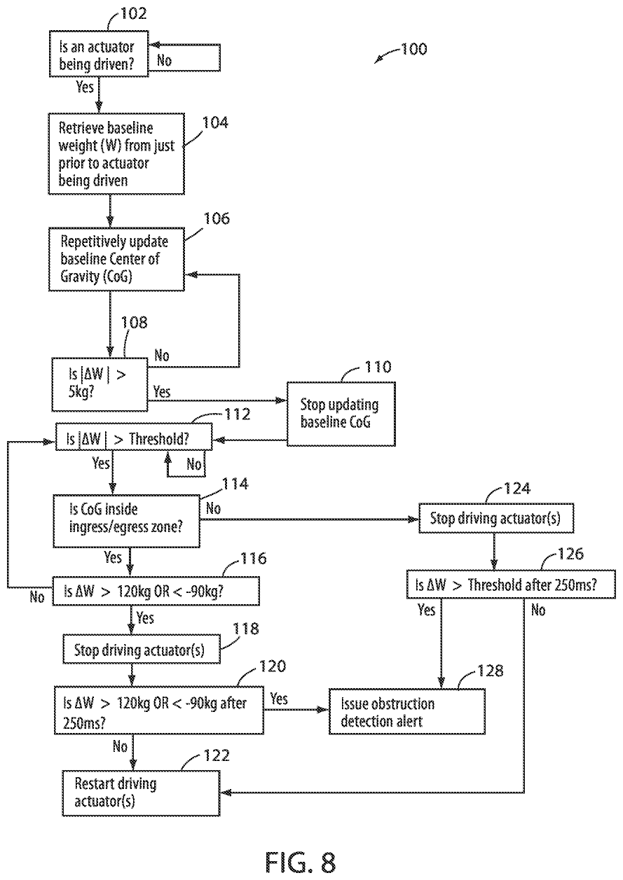

FIG. 8 illustrates one example of an obstruction detection algorithm 100 that may be executed by main controller 70 and/or motion controller 64. For purposes of brevity, the following description will be made with the assumption that main controller 70 carries out algorithm 100, but it will be understood that this is merely done for purposes of brevity, and that any one or more of the steps of algorithm 100 may be carried out by motion controller 64. It will also be understood that obstruction detection algorithm 100 may be varied in multiple different manners from the specific embodiment shown in FIG. 8, some of which will be discussed further herein.

Obstruction detection algorithm 100 is executed by controller 70 in order to automatically stop movement of one or more components of patient support apparatus 20 from being driven by actuators 26 and/or 66 when any of those moving components encounters an obstruction or obstacle. Obstruction detection algorithm 100 is therefore executed as a safety precaution in order to reduce the likelihood of damage to patient support apparatus 20 and/or any objects or people that may get in the way of (i.e. form an obstruction to) the moving component(s) of patient support apparatus 20. Obstruction detection algorithm 100 uses the outputs of force sensors 54 in order to determine whether an obstruction is, or was, encountered during movement of any of the components of patient support apparatus 20.

Obstruction detection algorithm 100 begins at a step 102 wherein controller 70 checks to see if any of the actuators 26a,b and/or 66a,b are currently being driven. The driving of actuators 26a,b and/or 66a,b are controlled by, as explained earlier, a person pressing on one of the movement controls 58 on any of control panels 56a-c (e.g. Fowler-up control 58n, Fowler-down control 580, etc.). If the answer is no, then controller 70 repeats step 102 and checks once again if any actuators 26a,b and/or 66a,b are being driven. In other words, controller 70 repetitively monitors the driving of actuators 26a,b and 66a,b to see if they are being driven, and it won't commence with the rest of obstruction detection algorithm 100 until one or more of these actuators is driven. This is because, unless one or more actuators 26a,b and/or 66a,b are being driven, there is no chance any components of patient support apparatus 20 will be driven into an obstruction, and therefore it is not necessary to perform the rest of algorithm 100.

Once controller 70 determines at step 102 (FIG. 8) that one or more actuators 26a,b and/or 66a,b are being driven, it moves to step 104. At step 104, controller 70 records the baseline weight reading from immediately prior to the actuator(s) 26, 66 being driven. That is, controller 70 is repetitively taking weight readings and determining a baseline weight reading. Unless an actuator 26, 66 is subsequently driven, controller 70 may discard these baseline weight readings (or use them for other purposes besides obstruction detection). If a user actives a movement control 58 and one or more actuators 26, 66 are driven, controller 70 retrieves the baseline weight reading that was determined just prior to the activation of the movement control. Controller 70 uses that baseline weight reading to determine if any changes are made to the baseline weight reading during movement of the one or more components that are being moved by the driven actuator(s) 26, 66. The baseline weight reading is generated from the outputs of force sensors 54. The outputs of each force sensor 54 are summed together to determine a current weight reading. These current weight readings may be generated multiple times a second, and controller 70 may be configured to filter out noisy transients in these weight readings in a number of manners. In general, controller 70 determines the baseline weight reading by averaging, or otherwise filtering, a number of successive weight readings that are taken during a time period when the readings from the load cells 54 are relatively stable.

After retrieving the baseline weight reading at step 104 (FIG. 8), controller 70 moves to step 106. At step 106, controller updates a baseline center of gravity calculating using the current force values from each force sensor 54. That is, controller 70 continues to take multiple readings a second (or another time period) from each force sensor 54 and to use those for calculating a center of gravity of the weight onboard patient support apparatus 20. From step 106, controller 70 proceeds to step 108 where it checks to see if a change in weight (from the baseline weight reading) has occurred that exceeds a first threshold. The change in weight may be either positive or negative. That is, controller 70 checks to see if a weight greater than the first threshold has been added to the load supported by the force sensors 54, or if a weight greater than the first threshold has been removed from the load supported by the force sensors 54. In some embodiments, this first threshold is set to approximately five kilograms. It will be understood, however, that the value of this first threshold may be varied considerably, both lower and higher. Indeed, in some embodiments, step 108 may be omitted from algorithm 100.

If controller 70 does not detect a change in weight (from the baseline weight retrieved at step 104) at step 108 that exceeds the first threshold, it returns to step 106 and continues to update the current center of gravity of the load supported by force sensors 54. If it does detect a change in weight that exceeds the first threshold at step 108, controller 70 moves to step 110 (FIG. 8). At step 110, controller 70 stops updating the baseline center of gravity calculation using the values from force sensors 54 and saves the most recent force values from force sensors 54 and/or the most recent center of gravity calculation. As will be discussed below, these force values and/or baseline center of gravity are subsequently used to compute the center of gravity of the added or reduced load detected by force sensors 54. After saving the most recent force values from force sensors 54 (and/or the most recent baseline center of gravity calculation) at step 110, controller 70 moves to step 112.

At step 112 (FIG. 8), controller 70 determines if the change in weight (from the baseline reading retrieved at step 104) exceeds a second threshold. The second threshold is larger than the first threshold used at step 108. In some embodiments, the second threshold is user-selectable and can vary, depending upon which values the user has selected for the second threshold. In some embodiments, if the user has selected a high sensitivity for the obstruction detection system (i.e. it will detect potential obstructions more easily), the second threshold may be set in the vicinity of thirty kilograms; and if the user has selected a medium sensitivity for the obstruction detection system, the second threshold may be set in the vicinity of sixty kilograms; and if the user has set a low sensitivity for the obstruction detection system, the second threshold may be set in the vicinity of ninety kilograms. Of course, in some embodiments, controller 70 is configured such that the second threshold is static and not adjustable by the user, in which case the second threshold may be set within the vicinity of any of these three values (or at another value).

Regardless of whether the second threshold is user-adjustable or not, controller 70 determines whether any change in weight from the baseline weight reading exceeds the threshold at step 112 (FIG. 8). If the weight change does not exceed the second threshold, controller 70 returns to step 112 and checks to see if any subsequent changes in the weight detected by force sensors 54 exceeds the second threshold. This continues until the user stops activating a movement control and the driving of actuator(s) 26 and/or 66 stops. In other words, controller 70 repetitively looks for changes in weight that exceed the second threshold throughout the movement of any of the components that are driven by any one or more of the actuators 26 and/or 66.

If controller 70 detects at step 112 that a change in weight greater than the second threshold has been detected, it moves to step 114 (FIG. 8). At step 114, controller 70 determines the center of gravity of the location of the weight change. This calculated center of gravity is of the center of gravity of the weight change, not the center of gravity of the overall weight supported by the force sensors 54. In order to compute the center of gravity of this weight change, controller 70 may utilize different methods. In a first method, controller 70 determines the current center of gravity and determines its weighted difference from the baseline center of gravity reading stored at step 110. Using the total weight change between the current weight and the baseline weight reading, as well as change in the current center of gravity with respect to the baseline center of gravity, controller 70 can determine where the change in weight must have occurred in order to yield the current weight and center of gravity.

Alternatively, controller 70 utilizes the current values from force sensors 54 and the values from force sensors 54 that were stored at step 110. For each force sensor 54, controller 70 determines the difference between the force sensor 54 reading stored at step 110 and the current force sensor reading (at step 112). This difference in force sensor readings for each force sensor 54 is then used to calculate a center of gravity of the change in weight. This center of gravity of the change in weight corresponds generally to the location at which an obstruction impacted patient support apparatus 20, or it corresponds to the location at which a weight was added to, or removed from, patient support apparatus 20. In other words, the change in weight of step 112 may result from either an obstruction being hit while one or more components of patient support apparatus 20 are moving, or it may result from a weight being added to, or removed from, patient support apparatus 20. The remaining steps of algorithms 100 are designed to automatically distinguish between changes in weight due to the addition or removal of objects (or people) from patient support apparatus 20 and changes in weight that are due to a moving component of patient support apparatus 20 impacting an obstruction. These steps will now be discussed in more detail.

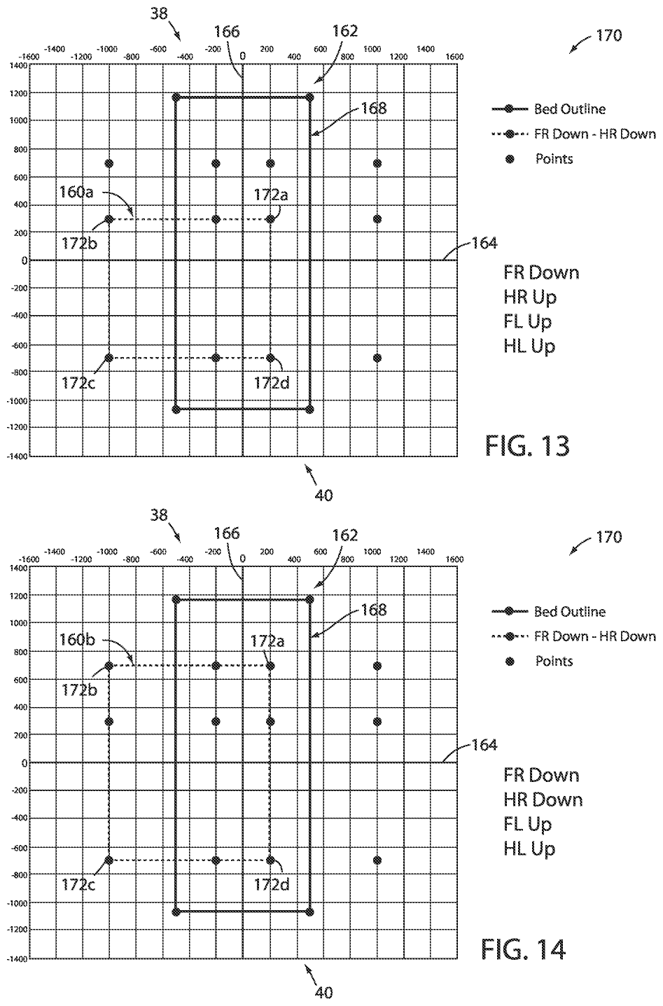

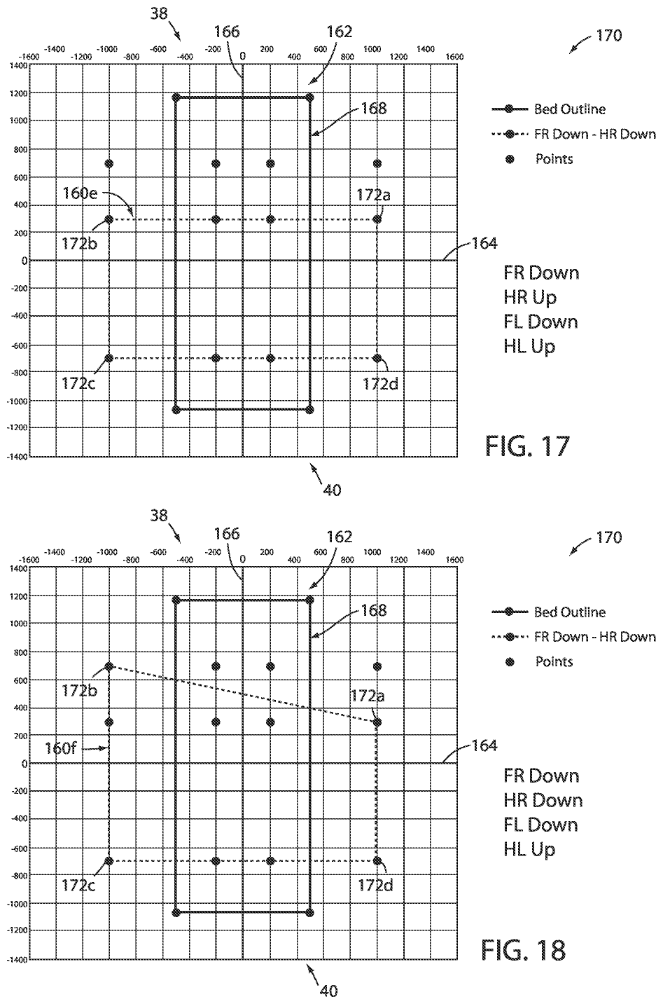

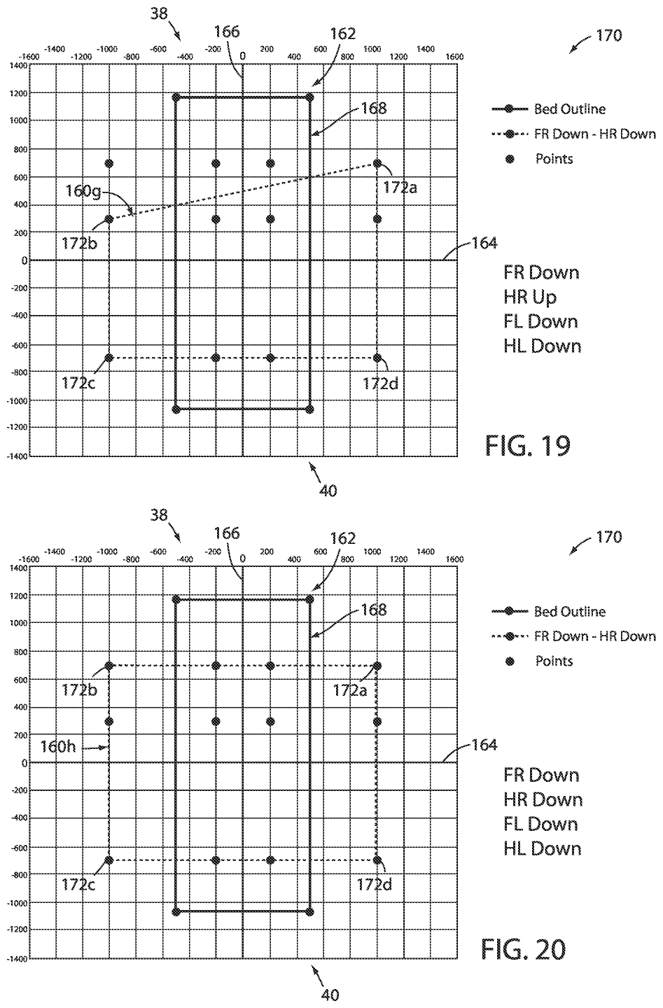

At step 114, controller 70 determines whether the location of the change in weight that exceeded the second threshold (at step 112) is inside or outside of an ingress/egress zone 160 (see FIGS. 13-20) or not. That is, controller 70 determines whether the center of gravity of the change in weight is inside or outside of the ingress/egress zone 160. The ingress/egress zone refers to an area of patient support apparatus 20 where a patient is likely to get into, or out of, patient support apparatus 20. As will be discussed in greater detail below with respect to FIGS. 13-21, the boundaries of the ingress/egress zone 160 may change, depending upon the current state of the siderails 36 (raised or lowered) and/or other factors. Generally, speaking, controller 70 uses the ingress/egress zone to determine whether the change in weight may have resulted from a patient getting into or out of patient support apparatus 20 during movement of patient support apparatus 20, rather than from an obstruction being hit during movement of the patient support apparatus 20.

If the center of gravity of the weight change is outside of the ingress/egress zone, controller 70 proceeds to step 124 where it automatically stops driving whichever actuator(s) 26 and/or 66 were being driven. This results in the stopping of the movement of any previously moving component of patient support apparatus 20. From step 124 (FIG. 8), controller 70 proceeds to step 126. At step 126, controller 70 waits for a predetermined amount of time after stopping the movement at step 124, and then re-checks the change in weight in comparison with the second threshold.

In other words, at step 126, controller 70 performs the same check performed at step 112. However, step 126 is performed with the then current readings from force sensors 54, rather than the weight readings that were used at step 112. If the change in weight still exceeds the second threshold as step 126, controller 70 concludes that an obstruction has been impacted and proceeds to step 128 where it issues an obstruction detection alert. If the change in weight has fallen below the second threshold at step 126, controller 70 concludes that the change in weight was a transitory weight fluctuation that was not the result of an obstruction, and therefore proceeds to automatically start (at step 122) re-driving whichever actuator(s) 26 and/or 66 that were previously being driven. In other words, controller 70 re-starts movement of the one or more components at step 122.

The purpose of step 126 is to help filter out changes in weight that may be due to transient variations in the load supported by force sensors 54 and/or changes in weight that only temporarily exceed the second threshold. This is done to help reduce the number of false positives detected by the obstruction detection system. In other words, it is done to help reduce the number of times that the obstruction detection system may conclude that an obstruction was impacted when, in fact, no obstruction was actually impacted. Whenever an obstruction is actually impacted during movement of a component of patient support apparatus 20, the change in the weight detected by force sensors 54 will remain after movement of the component stops. However, if an object (or person) weighing less than the second threshold is added to, or removed from, patient support apparatus 20, the change in the weight detected by force sensors 54 might transiently exceed the second threshold for a short amount of time due to the acceleration of the component(s) of patient support apparatus 20 where the object was added or removed. For example, if a twenty kilogram weight is dropped from a sufficiently high height onto patient support apparatus 20, it may cause the force sensors 54 to temporarily detect more than, say, sixty kilograms at the moment of impact of the object with patient support apparatus 20. Controller 70 therefore checks to see if the initial change in weight that exceeded the second threshold (at step 112) has persisted (at step 126) for the predetermined amount of time after movement has stopped. If it has, controller 70 concludes that the weight change is due to an obstruction. If it has not, controller 70 concludes that the weight change was not due to contact with an obstruction.

Although the predetermined amount of time used in step 126 in the example shown in FIG. 8 is 250 milliseconds, it will be understood that this is merely one example of the predetermined time period that may be used in step 126. In some embodiments, the predetermined time period may be shorter, while in other embodiments the predetermined time period may be longer. In general, the predetermined time period may range from less than a fourth of a second to several seconds. The general constraint on lengthening the predetermined time period is the potential negative impact on the user's experience of having a component of patient support apparatus 20 automatically stop movement for a lengthy amount of time when no actual obstruction has been detected. The general constraint on shortening the predetermined time period of step 126 is that the outputs of force sensors 54 may not have enough time to settle after an object weighing less than the second threshold amount is added to, or removed from, the patient support apparatus 20, therefore leading to the possibility that controller 70 incorrectly concludes that patient support apparatus 20 has impacted an obstruction when, in fact, it has not.

In an alternative embodiment, controller 70 executes step 126 by taking repetitive weight change readings during the entirety of the predetermined time period (e.g. 250 milliseconds), rather than by taking a single weight change reading at the expiration of the predetermined time period. In such embodiments, controller 70 compares each of the readings taken during the predetermined time period to the second threshold of step 112 and, if one of the readings falls below the second threshold, it proceeds to step 122 and re-starts driving the one or more previously driven actuators 26 and/or 66. If none of the readings taken during the predetermined time period fall below the second threshold, controller 70 moves to step 128 and issues an obstruction detection alert.

Returning to step 114 (FIG. 8), if controller 70 concludes that the center of gravity of the change in weight is inside of the ingress/egress zone, it moves from step 114 to step 116. At step 116, controller 70 compares the change in weight to a third threshold. In the particular example shown in FIG. 8, the third threshold has different values for negative and positive weight changes. Specifically, controller 70 checks to see if more than 120 kilograms has been added to the load supported by force sensors 54 or if more than 90 kilograms has been removed from the load supported by force sensors 54. If such a change in weight has been detected, controller 70 moves to step 118. If such a change has not been detected, controller 70 returns to step 112 and proceeds in the manner previously described.

The purposes of step 116 is to account for the possibility that a patient either may have exited patient support apparatus 20 or entered patient support apparatus 20 while a component of the patient support apparatus 20 was moving. Such a patient entry or exit may lead to changes in weight that exceed the second threshold of step 112, but such changes are not due to an obstruction being impacted. Accordingly, in order to help filter out false positives of obstruction detection that are due to a patient entering or exiting patient support apparatus 20, controller 70 uses a third threshold at step 116 if the center of gravity of the change in weight is inside of the egress/ingress zone. In other words, if the change in weight occurs in an area of patient support apparatus 20 where a patient is likely to enter or exit patient support apparatus 20, controller 70 uses a third threshold that is set at a higher level than the second threshold. The precise values of the third threshold of FIG. 8 in step 116 may, of course, vary (as may the first and second thresholds), but are generally selected to be large enough to account for typical patient weights being added to, or removed from, patient support apparatus 20, while being small enough such that damage to patient support apparatus 20 is unlikely to occur. In other words, the weight limits of step 116 may be selected to be large enough to account for typical patient weights, but small enough such that movement of patient support apparatus 20 will automatically be stopped by controller 70 before damage may occur to patient support apparatus 20.

With respect to being large enough to account for typical patient weights, it should be noted that typically when a patient is already positioned onboard patient support apparatus 20 while it begins moving, the patient will typically not completely exit from patient support apparatus 20 during such movement, but instead will typically only partially exit. The negative weight threshold of step 116 therefore only needs to be large enough to encompass a portion of a typical patient's weight. For example, consider a patient seated on patient support apparatus 20 as the lifts 26 are lowered toward the floor. As the lifts 26 are lowered, the patient's feet may come into contact with the floor (or the patient may start to put more or their weight on the floor as the lifts are lowered). These situations will lead to a portion of the patient's weight being offloaded from patient support apparatus 20 onto the floor, but a portion of the patient's weight will typically remain on patient support apparatus 20 until lifts 26 stop moving. Accordingly, the reduction in weight detected by force sensors 54 during the lowering of lifts 26 will be less than the total weight of the patient.

Similarly, consider the example of a patient seated on patient support apparatus 20 with his or her feet resting on the floor while the lifts 26 are raised. As the lifts 26 are raised, the portion of the patient's weight that is supported on the floor (through their feet) will decrease (and eventually may drop to zero if the patient's feet are moved out of contact with the floor). This will result in an increase in weight detected by force sensors 54. However, the increase in weight will be only a fraction of the patient's total weight because the patient was only partially supported on the floor before the upward movement of the lifts 26 began. Accordingly, the positive value of the third threshold of FIG. 8 does not need to be large enough to encompass the entire weight of patient. Instead, it can be selected to be a percentage of the typical, or expected weight, of a patient (i.e. an average patient weight, or a set amount over an average patient weight), and/or a percentage of the amount of patient weight that is currently supported on patient support apparatus 20 (which may be less than the total weight of the patient).

The use of higher force values for the third threshold of step 116 (when compared to the second threshold of step 112) does not negatively impact the ability of obstruction detection algorithm 100 to detect obstructions. This is because, when a moving component of patient support apparatus 20 impacts an actual obstruction, the change in forces detected by force sensors 54 due to the obstruction will typically tend to continue to increase for as long as the actuator(s) 26 and/or 66 are driven. Accordingly, if an actual obstruction is impacted, it will eventually lead to a change in weight that exceeds the higher values of the third threshold of step 116. In other words, using the higher third threshold of step 116 may delay the stopping of the actuator(s) 26 and/or 66 by a fraction of a second, but it will typically not cause the obstruction detection algorithm 100 to overlook an actual impact with an obstruction. Instead, it will typically filter out changes in force that exceed the second threshold of step 112 but that are due to partial patient ingress/egress, rather than actual contact with an obstruction.

At step 118 (FIG. 8), controller 70 automatically stops driving whichever actuator(s) 26 and/or 66 that were being driven at that moment. From step 118, controller 70 moves to step 120, which is the same as step 126. At step 120, as with step 126, controller 70 waits for a predetermined amount of time after the actuators 26 and/or 66 have stopped moving and re-checks the change in weight detected by force sensors 54 (compared to the baseline weight determined at step 104). If this re-checked change in weight also exceeds the third threshold, controller 70 concludes that an actual obstruction has been detected and proceeds to step 128. If the re-checked change in weight does not exceed the third threshold, controller 70 concludes that the change in weight that previously exceeded the third threshold (step 116) was only a transient reading, which is unlikely to have been caused by an obstruction being impacted, and therefore proceeds to step 122 where it automatically restarts driving the actuator(s) 66 that were previously being driven.

In some embodiments, after restarting movement at step 122, controller 70 returns to step 112 and continues to perform the subsequent steps in the manner previously recited until movement of patient support apparatus 20 stops, due to either the actuator(s) 26 and/or 66 reaching their desired state or the user stopping activation of the movement control. The movement control refers to any of controls 58 that, when pressed by the user, cause actuators 26 and/or 66 to move one or more components of patient support apparatus 20. Thus, for example, on the caregiver control panel 56b of FIG. 6, controls 58g-q are all movement controls. Once movement of patient support apparatus 20 has stopped due to the user deactivating the movement control, or due to the actuator(s) 26 and/or 66 reaching their desired position(s), algorithm 100 returns to step 102 and begins monitoring for subsequent activation of another movement control. When such subsequent movement is detected, controller 70 proceeds to step 104 and carries out the other steps of algorithm 100 in the manner previously recited.

It should be noted that, when controller 70 returns from step 122 to step 112, controller 70 continues to use the same second threshold value at step 122 as well as whatever weight change readings are current at that time. In other words, controller 70 does not reset the baseline weight reading (step 104). Controller 70 only resets the baseline weight reading when it returns to step 102 and proceeds to step 104 (after re-detecting the user activating a movement control). It does not reset the baseline weight reading after it automatically re-starts movement at step 122. Thus, for example, suppose the second threshold of step 112 is set to sixty kilograms and controller 70 detects a seventy kilogram weight change at step 112. Suppose further that the seventy kilograms weight change is detected outside of the ingress/egress zone and that, after the predetermined time period, only fifty of the seventy kilogram load is detected. Because fifty kilograms is less than the sixty kilogram second threshold, controller 70 moves from step 126 back to step 112. At that moment, the change in weight will still be fifty kilograms, and controller 70 will not reset this baseline weight. Accordingly, it will only take an additional ten plus kilograms to exceed the second threshold and cause controller 70 to move from step 112 to step 114.

Algorithm 100 is restarted at step 102 in response to any of the following three conditions: (1) motion of the actuator(s) 26 and/or 66 has stopped because the user has removed his or her finger from the corresponding movement control; (2) motion of the actuator(s) 26 and/or 66 has stopped because the actuator(s) 26 and/or 66 have reached their desired positions corresponding to the movement control (e.g. if the Trendelenburg control 58i is pressed, motion stops when the litter frame reaches the Trendelenburg position); or (3) motion of the actuator(s) 26 and/or 66 has stopped due to an obstruction being detected and controller 70 has proceeded to step 128. When algorithm 100 restarts at step 102, controller 70 waits for the next instance of driving one or more of the actuator(s) 26 and/or 66, and then proceeds to step 104 where it calculates a new baseline weight (and proceeds to the subsequent steps in the manner previously described).

It will be understood that algorithm 100 may be modified substantially from the specific example shown in FIG. 8. Some of these modifications are discussed explicitly below. Additional modifications not explicitly discussed below are also possible. In one such modification, algorithm 100 does not use a different threshold for a patient ingress/egress zone. That is, algorithm 100 may be modified to omit steps 114, 116, 124, and 126. When so modified, controller proceeds from step 112 to step 118 if a change in weight is detected at step 112 that exceeds the second threshold. In another possible modification, the thresholds of steps 108, 112, and/or 116 may be modified. In yet another possible modification, step 108 may be omitted, and controller 70 may proceed from step 106 directly to step 112 and use the baseline center of gravity reading at step 114 that corresponds to the center of gravity calculated the moment before the change in weight of step 112 was detected. In another modified embodiment, steps 120 and/or 126 may be omitted and controller 70 may be configured to proceed directly from step 118 or 124 to step 128.

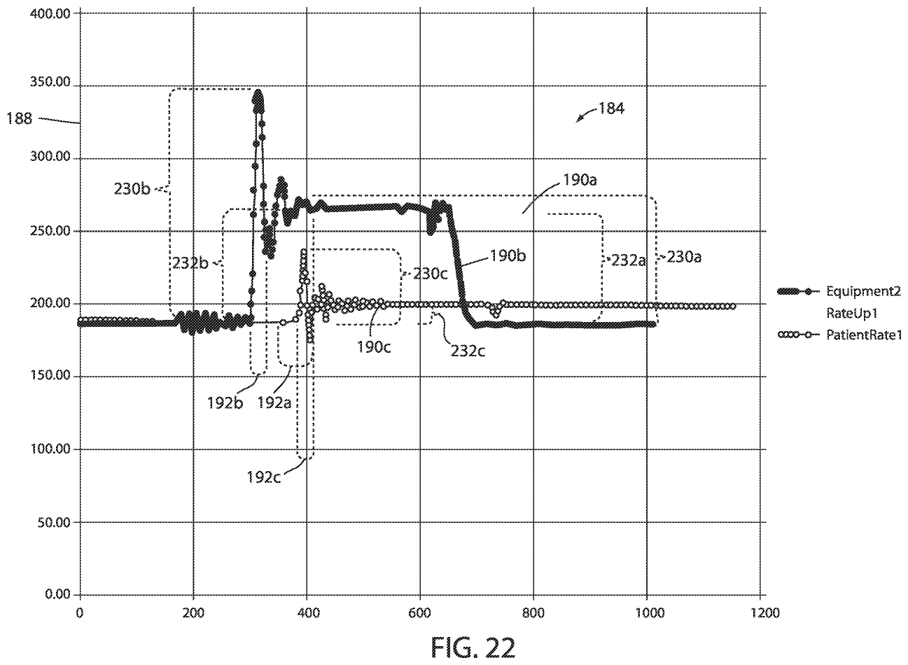

Still another modification of algorithm 100 is the analysis of the rate at which the weight change of step 112 and/or 116 occurred. This analysis of the rate of the weight change may be included to further help filter out weight changes that occur for reasons other than contact with an obstruction. This rate of change analysis is discussed in more detail below with respect to FIG. 22.

Additional changes to algorithm 100 include the modification of the size, shape, and/or location of the ingress/egress zone based upon which specific actuator(s) 26 and/or 66 is being moved at the time a potential obstruction is encountered. For example, in one modified embodiment, if only the gatch actuator 66a and/or only the Fowler actuator 66b is being moved, controller 70 may be configured to skip the ingress/egress zone analysis of step 114. In other words, when either of these actuators 66a and/or 66b are being moved (and no lift actuator 26 is being moved), controller 70 may proceed from step 112 directly to step 124 if a change in weight is detected that exceeds the second threshold.

In another modified version of algorithm 100, algorithm 100 may take into account the height of litter frame 28 when the change in weight above the second threshold (step 112) occurs. If the change in weight greater than the second threshold (as detected at step 112) occurs while litter frame 28 is at a high height, it is less likely that the weight change is due to a patient getting into or out of patient support apparatus 20, and therefore less likely that the change in weight is due to patient ingress or egress. In such cases, controller 70 may skip the ingress/egress zone analysis in the manner mentioned above.

As yet another alternative, controller 70 may look at the sign of the weight change and the current state of patient support apparatus 20 with respect to the patient (i.e. whether it is currently occupied by the patient or not). When patient support apparatus 20 is already occupied (as detected by the total load on force sensors 54), positive changes in weight, even if part of the ingress/egress zone, are likely not due to another patient getting into patient support apparatus 20 because one patient is already occupying patient support apparatus 20. Accordingly, the patient ingress/egress zone analysis of steps 114 and 116 may be skipped, and/or a smaller second threshold may be used at step 112 due the unlikelihood of the change in weight being due to patient ingress. If patient support apparatus 20 is unoccupied at the time movement of one or more components of patient support apparatus 20 commences, then reductions in weight will not be due to a patient exiting patient support apparatus 20 because the patient is not on patient support apparatus 20. Accordingly, algorithm 100 may be modified to use a smaller second threshold at step 112 and/or to skip the ingress/egress zone analysis if a reduction in weight is detected after movement commences on an unoccupied patient support apparatus 20.

It will be understood that, by using the outputs of force sensors 54, which do not directly contact the obstruction, obstruction detection algorithm 100 is able to detect obstructions that are encountered no matter where they are located with respect to the moving components of patient support apparatus 20. This is because wherever an obstruction contacts patient support apparatus 20, the result will be a change in the weight detected by the force sensors 54 of obstruction detection system. If an obstruction is encountered while a component of patient support apparatus 20 is moving upward, the total weight detected by force sensors 54 will increase (because the obstruction will add an additional downward force on the patient support apparatus 20). If an obstruction is encountered while a component of patient support apparatus 20 is moving downward, the total weight will decrease (because some of the weight support by the patient support apparatus 20 will be offloaded to the obstruction). Obstructions can therefore be detected whenever litter frame 28 is being moved upward or downward, whenever head section 42 is being moved upward or downward, and whenever the gatch is being moved upward or downward.

The ability to detect an obstruction anywhere on patient support apparatus 20 is due to the fact that the force sensors 54 are configured to support litter frame 28 and the only moving components of patient support apparatus 20 are the litter frame 28 and one or more sections of support deck 30 which are, in turn, supported on litter frame 28. As a result, if litter frame 28, or any components that is supported by litter frame 28 (e.g. support deck 30, siderails 36, an IV pole mounted on litter frame 28, etc.) hits an obstruction during movement of litter frame 28 and/or during movement of a second of support deck 30, the contact between the moving component and the obstruction will change the amount of force detected by force sensors 54. Thus, for example, if litter frame 28 is lowered and any portion of litter frame 28 (or any component support by litter frame 28) hits an obstruction while it is being lowered, some of the weight of the litter frame 28 will be offloaded onto the obstruction, and force sensors 54 will detect a decrease in the total weight they detect. Similarly, if litter frame 28 is raised and any portion of litter frame 28 (or any component supported by litter frame 28) hits an obstruction, the obstruction will add weight to the load supported by patient support apparatus, which will lead to an increase in the total weight detected by force sensors 54. The same is true if Fowler section 42 is raised into, or lowered onto, an obstruction. And the same is also true if the gatch (the joint between the thigh and foot section) is raised or lowered. Accordingly, the obstruction detection system is adapted to detect obstructions at any location on patient support apparatus 20.

In some embodiments of patient support apparatus 20, controller 70 is configured to allow a user to manually change the sensitivity level of the obstruction detection system. The sensitivity level refers to the size of the change in weight that must be detected while a component is moving before controller 70 concludes that an obstruction has been hit. At a low sensitivity level, a relatively large change in weight is needed to cause controller 70 to conclude that an obstruction has been hit (and to automatically stop movement of the component(s). At a high sensitivity level, a relatively small change in weight is needed to cause controller 70 to conclude that an obstruction has been hit (and to automatically stop movement of the component(s)). At a medium sensitivity level, a change in weight between the relatively high and relatively small amounts is utilized by controller 70 to determine when an obstruction has been hit.

In at least one embodiment, the sensitivity of the obstruction detection system may be modified by the user by navigating to a menu screen, such as the menu screen 130 of FIG. 9. In some embodiments, menu screen 130 is displayed in response to a user activating navigation control 58f (FIG. 4). Alternatively, or additionally, menu screen 130 may be displayed in response to other actions by the user. Menu screen 130 includes a home option 142a, an exit detection control 58a, a monitoring system control 58b, a scale control 58c, and a locks control 58e. Controls 58a, 58b, 58c, and 58e of menu screen 130 may be the same as controls 58a, 58b, 58c, and 58e of FIG. 4. Menu screen also includes several options 132, including a history option 132a, a night light option 132b, a settings option 132c, a service option 132d, a help option 132e, a pressure reduction option 132f, a turn option 132g, and a pulmonary option 132h. If the user presses on, or otherwise selects, the history option 132a, controller 70 is configured to display data gathered over prior usage of patient support apparatus 20, such as, but not limited to patient weight readings, actuator usage, etc. If the user presses on the night light option 132b, controller 70 is configured to display one or more controls for controlling an onboard night light (not shown).

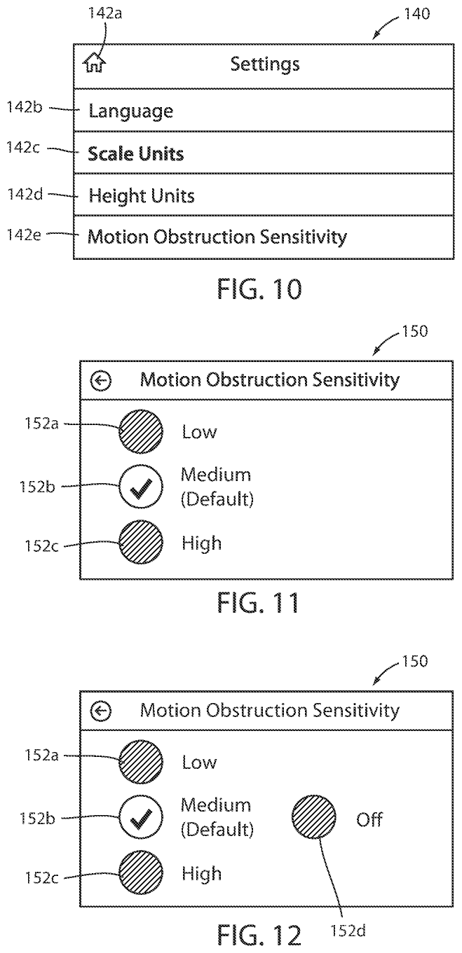

If the user presses on settings option 132c, controller 70 is configured to display a plurality of patient support apparatus settings that can be controlled by the user, such as, but not limited to, alert settings, motion control settings, etc. One example of a settings screen that may be displayed as a result of the user pressing on settings option 132c is the settings screen 140 of FIG. 10, which is discussed in greater detail below. If the user presses on service option 132d, controller 70 is configured to display a service screen that displays information relating to servicing needs and/or a service history of patient support apparatus 20. If the user presses on help option 132f, controller 70 is configured to display a help screen that includes additional information about how to operate patient support apparatus 20. If the user presses on any of pressure option 132f, turn option 132g, and/or pulmonary option 132h, controller 70 is configured to display one or more mattress control screens that include controls for carrying out pressure relief, turning, and/or pulmonary mattress therapy functions.

As noted, when a user presses on setting option 132c (FIG. 9), controller 70 is configured to display a settings screen, such as the settings screen 140 of FIG. 10. Settings screen 140 includes a home option 142a, a language option 142b, a scale units option 142c, a height units option 142d, and a motion obstruction sensitivity option 142e. When a user presses on home option 142a, controller 70 is configured to display a home screen on display 60, such as the screen shown in FIG. 4 and/or another home screen. When a user presses on language option 142b, controller 70 is configured to display different language options for displaying information on display 60 in different languages. When a user presses on scale units options 142c, controller 70 is configured to display a screen that allows the user to select between displaying weights in pounds or kilograms. When a user selects height units option 142d, controller 70 is configured to display a screen that allows the user to select between displaying the height of litter frame 28 in inches or centimeters. When a user selects motion obstruction sensitivity option 142e, controller 70 is configured to display an obstruction sensitivity screen, such as the obstruction sensitivity screen 150 of FIG. 11.

Obstruction sensitivity screen 150 (FIG. 11) includes a low option 152a, a medium option 152b, and a high option 152c. Each option 152a-c is selectable by a user and enables the user to select between high, medium, or low sensitivity levels for the obstruction detection system. In some embodiments, the sensitivity settings that the user selects on screen 150 changes the value of the second threshold used in step 112 (and step 126) of algorithm 100, but does not change the values used in step 116 (or step 120). In other embodiments, the sensitivity level selected by the user on screen 150 changes the values of both the second threshold used in steps 112 and 126 as well as the sensitivity levels used in steps 116 and 120 of algorithm 100. In one embodiment, the low sensitivity level option 152a corresponds to a value of approximately 90 kilograms for the second threshold used in steps 112 and 126; the medium sensitivity level option 152b corresponds to a value of approximately 60 kilograms for the second threshold used in steps 112 and 126; and the third sensitivity level option 152c corresponds to a value of approximately 30 kilograms for the second threshold used in steps 112 and 126. It will be understood that other values may be used for these sensitivity levels, and that other sensitivity options 132 may be presented to the user, in lieu of, and/or or in addition to, the options 152a-c shown in FIG. 11.

In some embodiments, controller 70 may be configured to utilize a value for the second threshold of steps 112 and 126 that is a percentage of the baseline weight, a percentage of the patient weight, a percentage of the empty weight (i.e. when no patient is present), or a percentage of another weight measured by force sensors 54. In those embodiments of the obstruction detection system that also change the third threshold used in steps 116 and 120 when the sensitivity level is changed, the third threshold may use values of greater than 120 kg or less than-90 kg for the low sensitivity level; values of greater than 90 kg or less than-60 kg for the medium sensitivity level; and values of greater than 60 kg or less than-30 kg for the high sensitivity level. Still other values may be used for the third threshold and such values may be predefined or they may be based on a percentage of the baseline weight, the patient weight, or another weight.

In some embodiments, patient support apparatus 20 is configured to allow a user to manually shut off the obstruction detection system. In such systems, obstruction sensitivity screen 150 may include an off option 152d, such as is shown in FIG. 12. In some such embodiments, it may be desirable to prevent a patient, or another unauthorized user, from turning off the obstruction detection system. In those situations, controller 70 may be configured to not display the off option 152d unless the user engages in a secret action that would not generally be known to non-authorized users. For example, in one embodiment, controller 70 is configured to not display the off option 152d unless the user simultaneously presses on two of the options 152a-c for a predetermined amount of time. As another example, controller 70 may be configured to not display the off option 152d unless the user presses on multiple ones of options 152a-c in a specified sequence. Still other secret actions may be required in order for controller 70 to display off option 152d, and such secret actions may utilize options 152a-c and/or one or more other controls 58 on patient support apparatus 20.

FIGS. 13-20 illustrate various shapes, sizes, and locations of an ingress/egress zone 160 that may be utilized as part of obstruction detection algorithm 100 (in step 114). As shown in these drawings, in at least one embodiment, the size, shape, and location of the ingress/egress zone 160 varies based upon the state of the four siderails 36 (up or down). Each of FIGS. 13-20 includes a graph 162 having an X-axis 164, a Y-axis 166, and a patient support apparatus boundary 168. The patient support apparatus boundary 168 corresponds to the outer perimeter of patient support apparatus 20 when looking down on patient support apparatus 20 from above. The units of the X and Y axes in FIGS. 13-20 are in millimeters. Accordingly, the patient support apparatus 20 example shown in these FIGS. 13-20) extends along the X-axis 164 from approximately negative 500 to positive 500, and therefore has a width of approximately 1000 millimeters (i.e. 1 meter). The patient support apparatus 20 shown in these FIGS. 13-20) extends along the Y-axis 166 from approximately negative 1100 to positive 1100, and therefore has a length of approximately 2600 millimeters (i.e. 2.2 meters). It will be understood that these are merely one example of the dimensions that a patient support apparatus 20 may be constructed with and that the principles of the present disclosure apply to patient support apparatuses 20 having other widths and/or lengths.

Each of FIGS. 13-20 includes a legend 170 indicating the state of each of the four siderails 36 for that particular figure. The legend 170 indicates which ones of the four siderails 36 are raised (up) and which ones are lowered (down). As can be seen from these figures, the size, shape, and/or location of ingress/egress zone 160 changes for each of the different combinations of the states of the four siderails 36. The abbreviation FR stands for Foot Right, which corresponds to the siderail 36 at the foot end 40 of patient support apparatus 20 that is positioned on the right side (from the patient's perspective, when he/she is lying on his/her back-note that the patient's right side, when lying on his/her back, is positioned on the left side of boundary 168 in FIGS. 13-20 from the viewer's perspective); the abbreviation HR stands for Head Right, which corresponds to the siderail 36 at the head end 38 of the patient support apparatus 20 and positioned on the right side; the abbreviation FL stands for Foot Left, which corresponds to the siderail 36 at the foot end 40 of patient support apparatus 20 and on the left side (from the patient's perspective, when lying on his/her back); and the abbreviation HL stands for Head Left, which corresponds to the siderail 36 at the head end 38 of patient support apparatus 20 and on the left side. Thus, for example, legend 170 of FIG. 13 indicates that all of the siderails 36 are in their upward, or raised, position with the sole exception of the foot end siderail 36 that is positioned on the patient's right side (when he/she is lying on his/her back).

In the situations shown in FIGS. 13-20, controller 70 uses an ingress/egress zone 160a-h that is a quadrilateral and has a first vertex 172a, a second vertex 172b, a third vertex 172c, and a fourth vertex 172d. In the specific example of FIG. 13, the first vertex 172a is positioned at approximately (200, 300); the second vertex 172b is positioned at approximately (−1000, 300); the third vertex 172c is positioned at approximately (−1000, −700); and the fourth vertex 172d is positioned at approximately (200, −700). Thus, at step 114 of algorithm 100, if all of the siderails 36 are up except for the one on the right side of patient support apparatus 20 and positioned toward foot end 40 (e.g. the situation of FIG. 13), controller 70 will use an ingress/egress zone 160a as shown in FIG. 13. That is, if the center of gravity of the weight change detected at step 112 is positioned inside of ingress/egress zone 160a, controller 70 will move to step 116. If not, it will move to step 124.

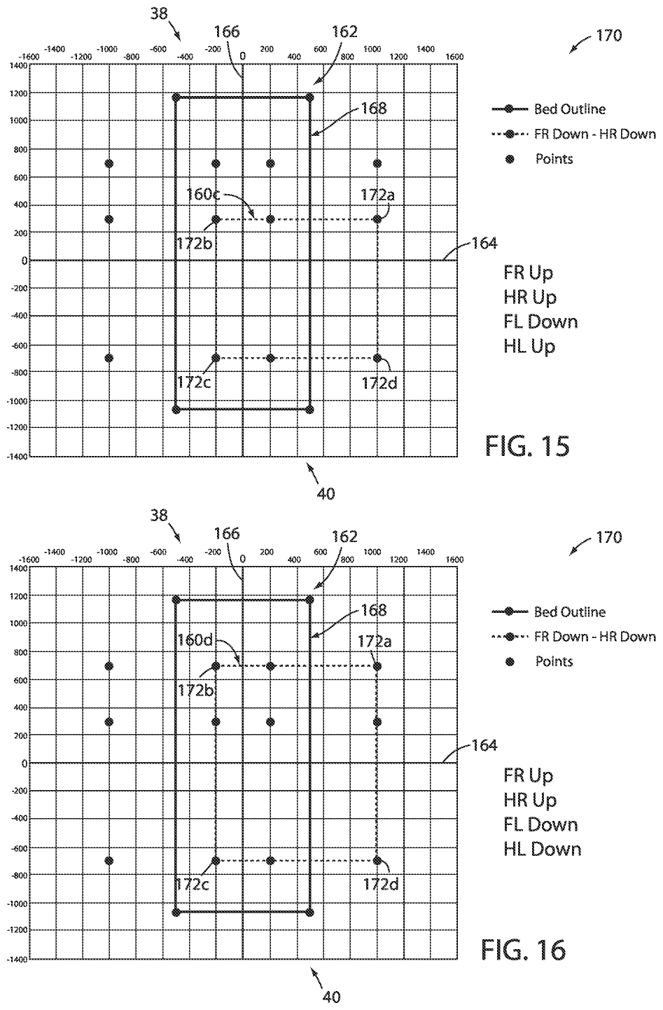

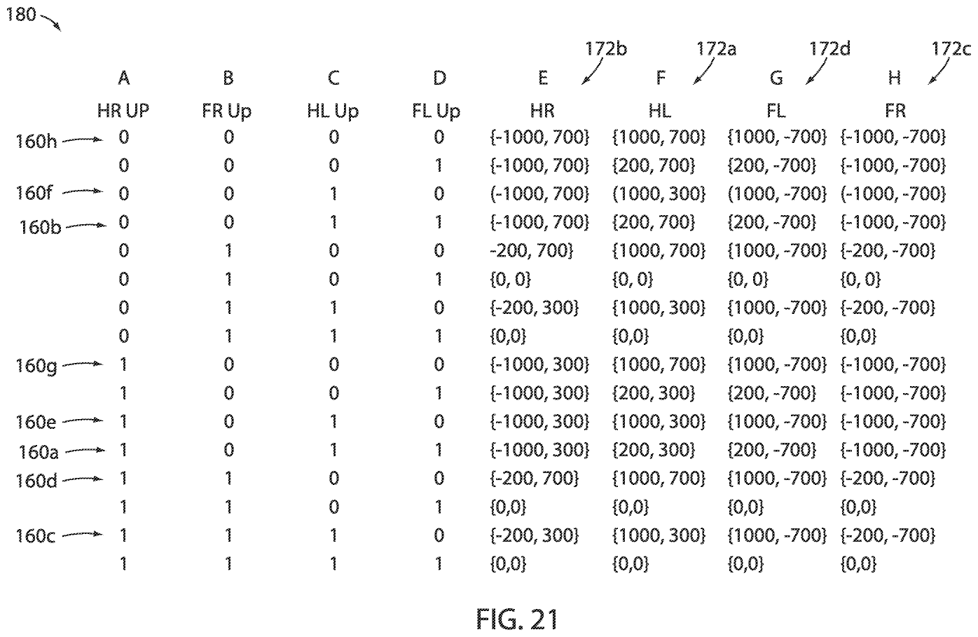

FIG. 14 illustrates the dimensions of an ingress/egress zone 160b when the front right siderail 36 is down, the head right siderail is down, and both the foot left and head right siderails 36 are up. Legends 170 of FIGS. 15-20 illustrate the corresponding locations and dimensions of the zones 160c-g corresponding to the different siderail statuses in each of these legends 170. Because there are four siderails 36, there are sixteen different combined states of these siderails. FIGS. 13-20 only illustrate eight of these combined states. Table 180 of FIG. 21, however, identifies the vertices 172a, 172b, 172c, and 172d for all sixteen of the different states of each of the siderails 36. That is, each row in table 180 corresponds to a different ingress/egress zone 160a, b, c, etc. Columns A, B, C, and D illustrate the state of each of the four siderails, with a zero corresponding to the logical state FALSE, and a one corresponding to the logical state TRUE. Thus, zone 160h corresponds to the situation where all four of the siderails are in their lowered position. Columns E, F, G, and H identify the coordinates of the locations of each of the four vertices 172 of the corresponding ingress/egress zone 160.

It will be understood that the size, shapes, and locations of the ingress/egress zones 160 may be changed from those shown in FIGS. 13-21. Such changes include, but are not limited to, zones 160 that are not quadrilateral shaped, zones 160 that include one or more curves along their boundaries, and/or zones 160 whose boundaries are dependent upon other factors besides the state of siderails 36 (e.g. the height of litter frame 28, the occupancy state of patient support apparatus 20, the actuator(s) 26 and/or 66 that are being driven, etc.).

FIG. 22 illustrates a graph 184 of the total weight sensed by force sensors 54 over time for different potential obstruction detection events. Graphs 184 includes an X-axis 186 and a Y-axis 188. X-axis 186 corresponds to time and Y-axis 188 corresponds to the total weight sensed by force sensors 54. Graph 184 illustrates three different plots 190a-c. The first plot 190a corresponds to the total weight readings from sensors 54 over time when one or more moving components of patient support apparatus 20 hit an obstruction. Plot 190b corresponds to the total weight readings from sensors 54 over time when a patient gets onto patient support apparatus 20 while one or more components of patient support apparatus 20 are moving. Plot 190c corresponds to the total weight readings from sensors 54 over time when an object, such as a piece of equipment, is added to patient support apparatus 20 while one or more components of patient support apparatus 20 are moving. In some embodiments, controller 70 is configured to analyze the rate of change of a plot 190 of the total weight readings from force sensors 54 over time to help determine if a change in weight is due to an obstruction or something else (e.g. equipment added or removed, or a patient entering or exiting patient support apparatus 20).

As can be seen in the examples shown in FIG. 22, the slope of the weight readings differs noticeably when an obstruction is impacted (plot 190a) versus when a piece of equipment is added to, or removed from, patient support apparatus 20 (plot 190c) or versus when a patient enters or exits patient support apparatus 20 (plot 190b). In particular, the rate of change of the weight readings is significantly higher for situations when equipment or a patient is added to, or removed from, patient support apparatus 20, as compared to when an obstruction is hit. In other words, the slope of plot 190a when an obstruction is first hit is less than the slope of plots 190b and 190c when an object or patient is added to, or off-loaded, from patient support apparatus 20. That is, the slope of plot 190a in region 192a is less steep than the slope of plot 190b in region 192b and the slope of plot 190c in region 192c. Accordingly, by monitoring the rate of change of the total weight sensed by sensor 54, controller 70, in some embodiments, is able to use the measured rate of change to determine if an actual obstruction has been hit, or if the change in weight is due to some other factor (e.g. a patient or equipment) that does not involve an obstruction.

In those embodiments of patient support apparatus 20 where controller 70 is configured to monitor the rate of change of the force readings from sensors 54 during movement to determine if a sudden change in weight is due to an obstruction, controller 70 may perform this analysis at any time between step 112 (FIG. 8) and steps 122 and/or 126 of algorithm 100. In some embodiments, controller 70 may perform an analysis of the slope of the weight change plot 190 prior to stopping the actuators at steps 118 or 124, and if the weight change has a slope that is too steep for an obstruction, controller 70 can continue to drive the actuator(s) 26, 66 and return to step 108, and/or to another step. In other embodiments, controller 70 may analyze the slope of the weight change after the actuator(s) 26 and/or 66 are stopped at step 118 and/or 124, and if the slope analysis indicates that no obstruction was detected, controller 70 may proceed directly to step 122 and restart the actuators, regardless of whether or not the thresholds at steps 120 or 126 are met.

In some embodiments, controller 70 may analyze the rate of change of the weight in different manners, depending upon whether an increase in weight is detected or a decrease in weight is detected. For example, it has been found that when an obstruction is hit above patient support apparatus 20 while a component is moving upward—which leads to an increase in the total weight—the total measured weight will change no faster than about 0.9 kilograms per millisecond. When an obstruction is hit below patient support apparatus 20 while a component is moving downward—which leads to a decrease in the total weight—the total measured weight will change no faster than about −1.1 kilograms per millisecond. When a patient or object is added to the patient support apparatus, the rate of change of the weight may be closer to 5 kilograms per millisecond. When a patient exits from patient support apparatus 20, or an object is removed, the rate of change of the weight may be in approximately −1.5 kilograms per millisecond. Accordingly, in some embodiments, controller 70 may be configured to conclude that, when an increase in weight is detected that occurs at a rate faster than, say, 2.0 kilograms per millisecond, the weight increase is not due to an obstruction. In some embodiments, controller 70 may be configured to conclude that, when a decrease in weight occurs at a rate faster than, say, −1.3 kilograms per millisecond, the weight decrease is not due to an obstruction. Of course, other rate thresholds may be used for analyzing the rate of change of the weight in order to distinguish weight changes due to obstructions from weight changes due to patient or objects.

In some embodiments, controller 70 may be configured to perform other analyses on the plot of the weight readings from force sensors 54 when a change greater than the second threshold (step 112) is detected, either in lieu of, or in addition to, the rate of change analysis discussed above. In such embodiments, controller 70 may be configured to analyze the wavelength and/or frequency of the oscillations in the weight readings, the magnitude of the oscillations, the level of dampening, and/or the number of oscillations that occur after the maximum weight change is detected. Additionally, or alternatively, controller 70 may be configured to analyze the rate of change of the weight change readings after the maximum weight change is detected. Still further, controller 70 may be configured to analyze the magnitude of the overshoot of the maximum weight change from the second threshold (of step 112). Any one or more of these factors may be considered, either alone or in combination, by controller 70 in determining whether the change in weight is due to an obstruction or due to some other cause (e.g. a patient or piece of equipment being added/removed). As can be seen from FIG. 22, in situations where an obstruction is encountered (plot 190a), there are relatively few oscillations after the maximum weight change is detected, and the oscillations have a both relatively small wavelength and relatively small amplitude. In contrast, where a patient enters the patient support apparatus 20 (plot 190b) or where equipment is added to the patient support apparatus 20 (plot 190c), the oscillations are greater in number and of a larger wavelength and amplitude. In some embodiments, controller 70 utilizes the number of oscillations, their wavelengths, and/or their amplitudes to help determine whether the change in weight is due to an obstruction rather than from other causes.

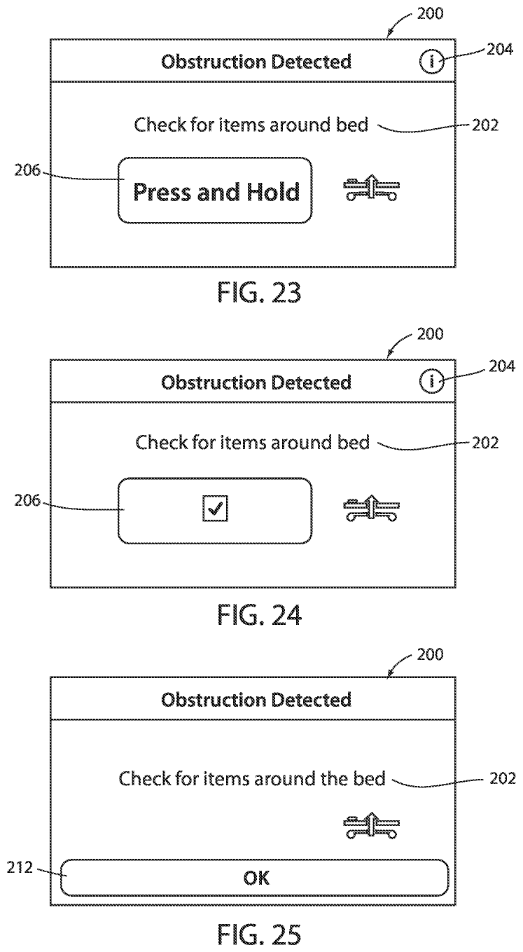

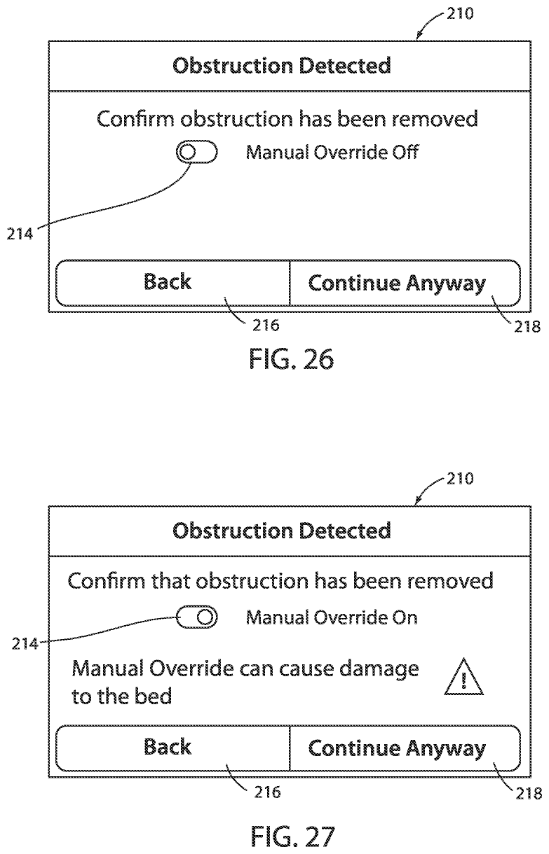

FIG. 23 illustrates one example of an obstruction detection warning screen 200 that may be displayed on display 60 when controller 70 determines that an obstruction was hit during movement of one or more components of patient support apparatus 20. In other words, controller 70 may be configured to display screen 200 at step 128 of algorithm 100 (FIG. 8). Warning screen 200 includes a message 202 advising the user to check for items around the patient support apparatus 20 that may have been impacted by movement of the patient support apparatus 20. Warning screen 200 also includes an information icon 204 and a recovery control 206. The information icon 204, when pressed by the user, causes controller 70 to display a manual override screen on display 60, such as the manual override screen 210 shown in FIGS. 26 and 27. The recovery control 206, when pressed and held by the user, causes controller 70 to automatically drive one or more of the actuator(s) 26 and/or 66 in a manner that relieves the pressure exerted by patient support apparatus 20 on the obstruction. In other words, the recovery control 206, when pressed by the user, causes controller 70 to drive the actuator(s) 26 and/or 66 such that the component of patient support apparatus 20 that came into contact with the obstruction is moved away from the obstruction.

In one embodiment, the recovery control 206 causes controller 70 to drive the one or more actuator(s) 26 and/or 66 for a predetermined amount of time (e.g. a second or two). Alternatively, in some embodiments, the recovery control 206 causes controller 70 to drive the one or more actuator(s) 26 and/or 66 for a predetermined amount of distance (e.g. an inch or two). In either embodiment, controller 70 is configured to drive the actuator(s) 26 and/or 66 for the predetermined distance or time only if the user continues to press and hold the recovery control 206. In other words, if the user stops pressing on recovery control 206 before the predetermined amount of time elapses or the predetermined distance is traveled, controller 70 will stop movement of the actuator(s).

Once the predetermined amount of time or distance is achieved using recovery control 206, controller 70 is programmed to both stop further movement of the actuator(s) 26 and/or 66 and to change the appearance of the recovery control 206, such as in the manner illustrated in FIG. 24. As shown in FIG. 24, the recovery control 206 has changed its message from “Press and Hold” to a check mark, indicating that the recovery movement has been completed. In addition, controller 70 may be configured to change the color of recovery control 206, such as from an amber color to a green color. Other variations may also be implemented.

In an alternative embodiment, controller 70 may be configured, when the user presses and holds the recovery control 206, to allow movement to continue beyond the predetermined time and/or distance mentioned above. In such embodiments, controller 70 may be configured to allow movement beyond the predetermined time and/or distance if controller 70 determines that such additional movement will not cause any of the actuator(s) 26 and/or 66 to move beyond a corresponding interference limit. The interference limits refer to positions of the actuators 26 and 66 beyond which controller 70 does not allow them to move. Such limits are implemented in order to prevent, for example, actuators 26 and/or 66 from moving in a manner that causes an end of litter frame 28 to potentially hit the ground, and/or in a manner that may cause components of patient support apparatus 20 to contact each other. So, for example, if litter frame 28 is level (i.e. generally horizontal) and an obstruction was detected while moving litter frame upward (e.g. pressing on control 58p; FIG. 6), controller 70 may be configured—in response to the user pressing and holding the recovery control 206—to allow lifts 26 to lower litter frame 28 all of the way to its lowest height position, rather than automatically stopping movement of the lifts 26 a predetermined time or distance after the recovery control 206 was activated. Controller 70 determines whether to allow such movement beyond the predetermined time or distance based on its preprogrammed interference limits, as well as the location at which the obstruction was detected, and whether the obstruction caused a negative or positive weight change.

After controller 70 has displayed the changed recovery control 206 shown in FIG. 24 for a predetermined amount of time (or, in some embodiments, after the user has released their finger from the changed recovery control 206 of FIG. 24), controller 70 is configured to automatically remove the recovery control from screen 200 and display only the message 202, such as is shown in FIG. 25. In addition, controller 70 is configured to display an OK option 212. The OK option 212 is pressed by the user after he or she has confirmed that the user has indeed checked to see if patient support apparatus 20 has collided with an obstruction, and if so, that the obstruction has been removed from the intended movement path of patient support apparatus 20. Once the user presses on the OK button, controller 70 is configured to stop displaying warning screen 200 and to return to displaying whatever screen was previously displayed prior to the detected impact with an obstruction. Alternatively, controller 70 may be configured to switch to displaying a different screen after the user presses on the OK option 212.

In some embodiments, while controller 70 is displaying the recovery control 206 on display 60, controller 70 is configured to disable all of the other motion controls on patient support apparatus 20, including those on each of the control panels 56a, 56b, and 56c. As a result, if a user tries to move patient support apparatus 20 out of contact with an obstruction using a movement control, such as by trying to press on, say, litter frame height control 58p or 58q (FIG. 6), controller 70 will not drive any actuator(s) 26 and/or 66 in response thereto. Consequently, in such embodiments, recovery control 206 becomes the only active movement control after an obstruction has been detected (unless the user overrides the obstruction detection, as will be discussed in greater detail below with respect to FIGS. 26 and 27). This forces the user to utilize the recovery control 206 to move patient support apparatus 20 out of contact with the obstruction. As will be discussed in greater detail below, the reason for temporarily disabling the other movement controls after an obstruction has been detected is because the proper movement control that should be activated in order to move the patient support apparatus 20 out of contact with the obstruction is not always obvious to the user, and in some cases, may cause one or more of the actuator(s) 26 and/or 66 to drive the component further into the obstruction. Controller 70 is configured to re-enable all of the movement controls on all of the control panels 56 after the recovery control 206 has been fully activated (i.e. after the user has pressed and held the recovery control 206 sufficiently long for controller 70 to have switched to displaying the recovery control 206 in the changed manner shown in FIG. 24). In other words, once the recovery control 206 has been activated to drive the actuator(s) 25 and/or 66 for the predetermined amount of time or distance, controller 70 automatically re-enables all of the movement controls on control panels 56a-c.

As was noted, if the user presses on the information icon 204 of screen 200 (FIGS. 23 and 24), controller 70 is configured to display an override screen 210, such as the override screens 210 shown in FIGS. 26 and 27. Override screen 210 includes a slider bar 214, a back option 216, and a “continue anyway” control 218. Override screen 210 is displayed in order to give the user the option of having to use recovery control 206. When a user slides slider bar 214 to the left (as shown in FIG. 26), controller 70 deactivates the manual override, and when the user slides slider bar 214 to the right (as shown in FIG. 27), controller 70 activates the manual override. When the manual override is active and the user presses the “continue anyway” control 216, controller 70 enables all of the motion controls on control panels 56a-c, even if the recovery control 206 has not been activated. When the manual override is not active, controller 70 temporarily disables all of the motion controls on control panels 56a-c until the recovery control 206 has been activated, as discussed above. Override screen 210 also includes a back control 216. When the user presses on the back control 216, controller 70 returns to displaying the warning screen 200 (FIGS. 23-25) that was previously displayed prior to the information control 204 being pressed.

If the user has activated the manual override slider bar 214 and pressed on the “continue anyway” control 218, controller 70 is configured to return to displaying whatever screen that was displayed on display 60 immediately prior to the obstruction being detected. Also, as noted, controller 70 is configured to re-enable all of the movement controls on all of the control panels 56a-c. Accordingly, if the user runs into an obstruction while, say, raising litter frame upwards using control 58p (FIG. 6) and an obstruction is detected, but the manual override is active and the user presses the “continue anyway” control 218, the user is thereafter free to re-press the upward control 58p, at which point controller 70 will drive lifts 26 in a manner that raises the height of litter frame 28. In this situation, the user has skipped utilizing the recovery control 206. Once such upward movement starts again, controller 70 will re-start algorithm 100 and proceed to step 102 where it takes a new baseline weight reading, and then proceeds in the manner previously discussed. If an obstruction was actually present and the user has not removed it, controller 70 will drive the lifts 26 further upwards until the increase in force due to thus additional driving of the litter frame 28 (or other component of patient support apparatus 20) into the obstruction reaches the second threshold (step 112), at which point controller 70 will run through the additional steps of algorithm 100 and eventually stop the movement again at steps 118 or 124. On the other hand, if no obstruction was actually present, the upward driving of the lifts 26 should not trigger another obstruction detection by algorithm 100, and the upward movement of the litter frame 28 should continue until the litter frame reaches the user's desired height.

In some embodiments, algorithm 100 may be modified such that controller 70 also compares the magnitude of the weight change of step 112 to an absolute reference value, and if the magnitude exceeds the absolute reference value, controller 70 may be configured to disable the override function. In other words, in some embodiments, if the total force exerted on the patient support apparatus 20 by the obstruction (whether a positive or negative force value) exceeds a set amount, controller 70 may be configured to disable the override function, and thereby force the user to utilize the recovery control 206. This helps prevent the user from damaging the patient support apparatus 20 by repeatedly using the recovery control 206 when an actual obstruction is present, which can lead to greater and greater amounts of obstructive force being applied to the patient support apparatus 20, which may eventually reach a level that can damage the patient support apparatus 20. In order to prevent this, controller 70 may look at the total weight being applied to patient support apparatus 20 and/or the total magnitude of the change in weight from an earlier baseline weight reading and, if it exceeds the set amount, it may temporarily disable the override function. Such temporary disabling of the override function may also be carried out by controller 70 in response to detecting multiple obstructions within a given time period, and/or in response to detecting multiple obstructions in response to activating the same movement control. By temporarily disabling the override function after multiple obstructions are detected, controller 70 helps prevent the situation where say, after a first obstruction detection event is detected and overridden, the initial obstruction weight change of 90 kilograms is increase to 180 kilograms, and then perhaps the override function is repeated, and the obstruction may end up exerting 270 kilograms, and so on. In other words, repeatedly using the override function in the presence of an actual obstruction can lead to ever increasing amount of force applied to one or more components of patient support apparatus 20. By detecting such repeated uses of the override control 214, controller 70 may be configured to disable the override control 214 before potential damage to patient support apparatus 20 occurs.

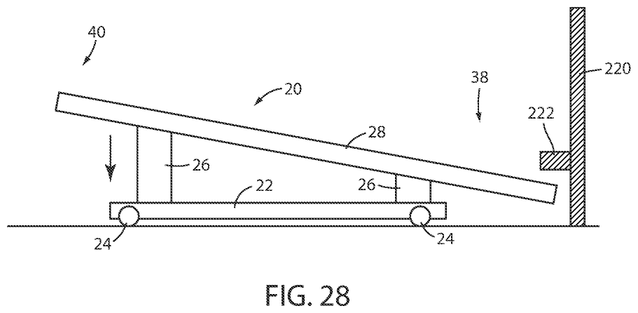

In some embodiments, controller 70 is configured to control actuators 26 and/or 66 in response to a user activating recovery control 206 in a manner that is different from merely reversing the previous movement of actuator(s) 26 and/or 66. This is because, in some situations, merely providing the opposite command to patient support apparatus 20 may lead it to move further toward the obstruction. This may be better understood with respect to the example shown in FIG. 28. FIG. 28 shows an elevation view of a diagram of patient support apparatus 20 positioned adjacent to a wall 220. Wall 220, in this example, includes a ledge 222 (or other structure that extends outwardly therefrom toward patient support apparatus 20). Litter frame 28 of patient support apparatus 20 is shown in FIG. 28 in the Trendelenburg position. That is, the head end 38 is positioned lower than the foot end 40.

If a user were to press on the litter lowering movement control (e.g. control 58q of FIG. 6 or control 58cc of FIG. 7), the user expects the patient support apparatus 20 to lower the height of litter frame 28. However, because litter frame 28 is tilted such that its head end 38 is positioned near to the floor, controller 70 is configured, in at least some embodiments, to react to the activation of a litter lowering movement control (e.g. 58q or 58cc) by instructing the head end lift 26 to first raise the head end 38 of litter frame 28 so that litter frame 28 becomes more horizontal, and then, after litter frame 28 is more horizontal, to simultaneously lower both the head end and foot end lifts 26 so that the overall height of litter frame 28 is reduced. Controller 70 does this so that head end of the litter frame 28 does not come into contact with the floor or come closer to the floor than is desired. As can be seen from the situation depicted in FIG. 28, however, as controller 70 raises the head end lift 26 in response to the initial activation of a litter frame lowering control (58q or 58cc), the head end of litter frame 28 may come into contact with ledge 222. When this happens, the head end lift 26 may continue to drive the head end 38 of litter frame into contact with ledge 222 until the force sensors 54 detect an increase in weight that is greater than the second threshold of step 112 (FIG. 8), at which point controller 70 will automatically stop movement of the litter frame 28.

In an alternative embodiment, controller 70 is configured to react to the activation of a litter lowering movement control (e.g. 58q or 58cc) by instructing the foot end lift 26 to first lower the foot end 40 of litter frame 28 so that litter frame 28 becomes more horizontal, and then, after litter frame 28 is more horizontal, to simultaneously lower both the head end and foot end lifts 26 so that the overall height of litter frame 28 is reduced. This motion too, however, may lead to head end 38 of litter frame 28 bumping into ledge 222. This is because, as foot end lift 26 is lowered, litter frame 28 will tend to pivot about a pivot axis located generally near where it is supported on the head end lift 26, and this pivoting will move the head end 38 upward toward ledge 222 (i.e. in the direction of the curved, upwardly pointing arrow of FIG. 28).

Regardless of whether controller 70 first lowers foot end lift 26 or first raises head end lift 26 in response to the user pressing on the litter lowering control (such as 58q or 58cc or another such control on control panel 56a), because the user was pressing on the litter lowering control when the litter frame 28 hit the obstruction 222, the user might conclude that pressing on the opposite movement control—a litter raising control such as 58p or 58bb or another similar control on control panel 56a—would resolve the contact with the obstruction 222. In this particular case, however, pressing on the litter raising control would only cause controller 70 to simultaneously raise both of lifts 26, thereby driving the head end 38 of the litter frame 28 into further contact with the ledge 222. Accordingly, in some situations, such as that shown in FIG. 28, pressing a raising control (such as controls 58l, 58n, or 58p of FIG. 6) in response to an obstruction being encountered during the pressing of a lowering control (such as controls 58m, 580, or 58q of FIG. 6) can lead to one or more actuator(s) 26 and/or 66 driving a component of patient support apparatus 20 further into the obstruction. This is the reason why controller 70 presents recovery control 206 (FIGS. 23-24), which, when activated by the user, automatically moves patient support apparatus 20 in a manner that will relieve pressure on the obstruction. This is also the reason why, if the user has not activated the manual override, controller 70 temporarily disables all (or a subset, as discussed further below) of the motion controls on control panels 56a-c until the user has finished activating the recovery control 206.

It will be noted that recover control 206 is displayed on display 60 of patient support apparatus 20, and that display 60 is located on footboard control panel 56a. In some modified embodiments, one or more of the siderail control panels 56b and/or 56c may be modified to include a display 60, and in such embodiments, controller 70 may be configured to display the recovery control 206 on the display(s) on the siderail control panels 56b and/or 56c (as well as on the display 60 of footboard control panel 56a, if footboard control panel 56a still includes a display in that particular embodiment). In this manner, if a user happens to be using a movement control on, say, siderail control panel 56b and an obstruction is encountered, he or she does not need to move over to the footboard control panel 56a to use the recovery control 206. Instead, the recovery control 206 will be displayed on the display 60 of the same siderail control panel 56b that the user is using, thereby allowing the user to activate the recovery control 206 without having to change position.

In another alternative embodiment, instead of adding a display 60 to one or more of the siderail control panels 56b and/or 56c, controller 70 may be configured to temporarily reassign one or more of the controls 58 on one or more of the siderail control panels 56b (or 56c) to implement the recovery control 206. In other words, when an obstruction is detected and motion stops, controller 70 may temporarily change the function of one or more of the controls 58 on the siderail control panels 56b and/or 56c such that the one or more control 58, when activated, carry out the function of the recovery control 206. In this manner, the user does not have to move to a position adjacent the footboard control panel 56a in order to activate the recovery control 206. Instead, he or she can continue to use the siderail control panel 56b (or 56c) to remedy the contact with the obstruction without having to relocate themself to the foot end 40 of patient support apparatus 20.

In the aforementioned alternative embodiment, controller 70 may temporarily re-assign the same one of the controls 58 of the siderail control panels 56b and/or 56c to the function of the recovery control 206. Alternatively, in some embodiments, controller 70 may temporarily re-assign a different control 58 on the siderail control panel the function of the recovery control, depending upon what movement was occurring when the obstruction was encountered, where the location was encountered, whether the obstruction added or removed weight, and/or what movement control 58 was being activated when the obstruction was impacted. For example, if some embodiments, if an obstruction is detected that results in an increase in weight, controller 70 may temporarily re-assign the lift lowering control 58q (and/or 58cc) to carry out the recovery function. In such embodiments, if an obstruction is detected that results in a decrease in weight, controller 70 may temporarily re-assign the lift raising control 58p (and/or 58bb) to carry out the recovery function. When either of these controls (lift lowering or lift raising) is temporarily re-assigned to perform the recovery function, controller 70 is configured—in response to one of these controls being activated—to simultaneously move both actuators 26 for the predetermined distance or the predetermined amount of time, as discussed above with respect to the recovery control 206. After the predetermined distance is moved (or the predetermined amount of time passes), controller 70 automatically reassigns the lift lowering or lift raising control 58 back to its original function.

The temporary re-assignment of the lift raising or lift lowering controls 58 discussed above may be better understood with respect to FIG. 28. As was discussed previously, if the user presses on the lift lowering control 58q or 58cc when litter frame 28 is in the position shown in FIG. 28, controller 70 is configured to either first lower foot end lift 26 or to first raise head end lift 26 a set amount of distance, and to thereafter simultaneously lower both lifts 26 until either the user stops activated the lift lowering control or the litter frame 28 reaches its lowest position. This is the normal function of lift lowering controls 58q and 58cc. When either of these controls are temporarily reassigned to perform the function of the recovery control, controller 70 is configured to—in response to the user activating one of these controls—simultaneously drive both of the lifts 26 downward for the predetermined time or distance discussed above. After reaching the predetermined time or distance, controller 70 automatically reassigns the control back to its normal function. When carrying out the recovery function 206, the lift lowering control 58q or 58cc causes both lifts 26 to be simultaneously driven downward immediately in response to their being activated. When carrying out their normal function, as noted above, the lift lowering control 58q or 58cc first causes one of the lifts to be driven until the litter frame 28 is more level, and then both of the lifts 26 to be simultaneously driven downward. It can therefore be seen that the lift lowering control 58q and/or 58cc can have its function temporarily reassigned in response to detecting an obstruction. A similar type of functional reassignment may be carried out by controller 70 for the lift raising controls 58p and/or 58bb when an obstruction is detected that causes a decrease in weight. In still other embodiments, one or more of the other controls 58 on the siderail control panels 56b and/or 56c may have their function temporarily changed to perform the same function as the recovery control 206 when an obstruction is detected.

Although not illustrated in algorithm 100, in some embodiments, controller 70 is configured to determine the maximum weight change detected in the moments before it stops the one or more actuator(s) 26 and/or 66 in response to a detecting contact with an obstruction. In addition, controller 70 is configured to determine the residual amount of weight change, which is the amount of weight change (from the baseline weight) that is detected by force sensors 54 in the moment after movement of the one or more actuator(s) 26 and/or 66 stops (or a predetermined amount of time after movement stops). In such embodiments, controller 70 may be configured to compare the maximum weight change to the residual weight change and use this comparison to help filter out false detections of obstructions. Generally speaking, if an obstruction is hit while a component of patient support apparatus 20 is moving, the residual weight change and the maximum weight change will generally be the same. On the other hand, if an object (or patient) is added to, or removed from, patient support apparatus 20 during movement of a component, the residual weight change will likely be significantly less than the maximum weight change detected during movement of the component. Therefore, in some embodiments, if controller 70 detects a significant drop in the residual weight change from the maximum weight change, it may be programmed to automatically re-start movement of the one or more actuator(s) 26 and/or 66 (after having stopped them at step 118 or 124).

The differences between the residual weight change and the maximum weight change for true obstructions versus the addition or removal of objects (or patients) can be better seen with respect to FIG. 22. As shown therein, the maximum weight change 230 is shown for an obstruction being hit (230a) for an object being added to the patient support apparatus 20 (230c), and for a patient entering patient support apparatus 20 (230b). The residual weight change 232 is also shown for the obstruction being hit (232a), for the object being added (232c), and for the patient entering patient support apparatus 20 (232b). As can be seen, the difference between the maximum weight change 230a for an obstruction being hit and the residual weight change 232a for an obstruction being hit is relatively small (about 10 to 20% of the residual weight change 232a). However, the difference between the residual weight change 232 and the maximum weight change for the object being added (230c and 232c) and for the patient entering patient support apparatus 20 (230b and 232b) is significantly larger. In the case of the patient entering patient support apparatus 20, the maximum weight change 230b is roughly twice the size of the residual weight change 232b). Similarly, in the case of an object being added to patient support apparatus 20, the maximum weight change 230c is more than 100% larger than the residual weight change 232c. Accordingly, in some embodiments, controller 70 may be configured to compare the maximum weight change to the residual weight change after automatically stopping movement of patient support apparatus 20, and if the difference is relatively small (e.g. the maximum weight is no more than 20 to 50% greater than the residual weight, although other thresholds may be used), controller 70 automatically concludes that an obstruction has, in fact, been hit. On the other hand, if the difference is larger than the selected threshold, controller 70 may be configured to conclude that no obstruction was, in fact hit, and therefore it may automatically re-start movement of the patient support apparatus 20.

As another example, in some embodiments, controller 70 may be configured to determine what percentage of the maximum weight remains when motion stops (i.e. what percentage of the maximum weight change the residual weight change is), and to compare that percentage to a threshold to determine if an actual obstruction was impacted or not. For example, in one embodiment, controller 70 may be configured to conclude that an actual obstruction was hit if the residual weight change is more than ninety percent of the maximum weight change, but no obstruction was impacted if the residual weight change is less than ninety percent of the maximum weight change. Other percentage thresholds greater than ninety percent or less than ninety percent can, of course, be used.

In some embodiments, controller 70 is may be configured to filter out false obstruction detection events by comparing the residual weight change to the second threshold used at step 112 of the patient support apparatus 20. If the residual weight change is greater than the second threshold by more than a defined amount (another threshold), controller 70 may be configured to conclude that no obstruction was actually hit. In such embodiments, the defined amount may be based upon the known speeds of the components of the patient support apparatus 20, the known amount of time it takes for controller 70 to automatically stop movement of patient support apparatus 20 when the weight change exceeds the second threshold (as determined at step 112), and an estimate of the likely amount of additional weight change that may result from a collision with an obstacle in the interim period of time between controller 70 determining that the second threshold was exceeded at step 112 and stopping movement at step 118 or 124. For example, if controller 70 is able to stop motion relatively quickly after the second threshold is breached at step 112 such that, a true obstruction will typically only increase the magnitude of the weight change by, say, 30 kilograms, then controller 70 may be configured to automatically conclude that any residual weight changes that are more than thirty kilograms greater than the second threshold used at step 112 are the result of a patient (or other relatively large weight) being added to patient support apparatus 20, rather than an obstruction being hit. Thus, for example, if the sensitivity level of the exit detection system is set such that the second threshold is thirty kilograms and a 100 kilogram patient enters patient support apparatus 20 during movement of the patient support apparatus 20, the residual change in weight will be 100 kilograms, which is seventy more than the second threshold. In such instances, the seventy kilogram increase in weight is significantly more than what is normally possible to be added by an actual obstruction in the interim period between the detection of a weight change greater than the second threshold at step 112 and the moment motion stops at steps 118 or 124 of algorithm 100. Accordingly, controller 70 may be configured in such situations to conclude that the seventy kilogram increase in weight over the second threshold of step 112 could not have been caused by an actual obstruction, but instead must have been caused by the addition of a patient (or other relatively large weight).

In some embodiments, the comparison of the maximum weight change versus the residual weight change, and/or the comparison of the residual weight change versus the second threshold used in step 112 (of algorithm 100) may be carried out in lieu of the rate of change analysis of the weight change discussed above (also with respect to FIG. 22). In other embodiments, controller 70 may be configured to analyze both the rate at which the change in weight occurs and the difference between the residual weight change and the maximum weight change (and/or the second weight threshold of step 112). In such embodiments, controller 70 may be configured to conclude that no obstruction has been hit only if both the rate of change and the difference between the maximum and residual weight changes indicate that no obstruction was hit. In still other embodiments, controller 70 may implement algorithm 100 in the manner shown in FIG. 8 without analyzing either the rate at which the weight change occurs or the difference between the maximum and residual weight changes (and/or the difference between the residual weight change and the second threshold of step 112).

In an alternative embodiment, instead of determining the maximum weight change that occurs while an actuator 26 and/or 66 is being driven, controller 70 is configured to determine the maximum weight change that occurs while the user continues to press a movement control 58. This may result in a different maximum weight reading because, after the one or more actuator(s) 26 and/or 66 are stopped being driven, the moving components of the patient support apparatus 20 may continue further movement due to momentum. In other words, motion may not completely stop at the moment driving signals to the actuator(s) 26 and/or 66 terminate, but instead may continue for a short moment afterwards. Further, this additional movement may result in weight changes that have a higher maximum than any of the weight changes detected while the actuator(s) 26 and/or 66 were being driven. Accordingly, by detecting the maximum weight change during the time period that the user continues to hold the movement control, a maximum weight change that occurs after the actuators are no longer being driven will be detected. This maximum weight change is then compared to the residual weight change in the manner mentioned above (the residual weight change may still be taken at the moment the actuator(s) cease to be driven, or a predetermined amount of time thereafter).

In some embodiments, controller 70 is configured to log every potential obstruction detection event. That is, in some embodiments, controller 70 is configured to record a number of values every time it detects a potential obstruction event. A potential obstruction event refers to every time controller 70 detects a change in force at step 112 (FIG. 8) that exceeds the second threshold. For each potential obstruction event, controller 70 may be adapted to record the weight changes detected during that event (while executing algorithm 100), the sensitivity level that was in use during the event, and a true or false conclusion after the event as to whether an obstruction was, indeed, actually hit during movement of the component(s) of patient support apparatus 20. In some such embodiments, controller 70 concludes that an obstruction was in fact hit (i.e. a true collision event) when it issues an obstruction alert at step 128 and the user does not utilize the manual override function (discussed above). If the user uses the manual override function, or if controller 70 proceeds to step 122 and automatically re-starts movement of the one or more actuator(s), controller 70 may be configured to conclude that no actual obstruction was hit during that potential collision event (i.e. it was a false collision event). As noted, controller 70 is also configured in some embodiments to record what sensitivity level was being used for each potential collision event (i.e. each time controller 70 detects a change in weight that exceeds the second threshold at step 112).

After each potential collision event, controller 70 is configured to determine from the weight changes recorded during that event whether or not controller 70 would have changed its true or false conclusion regarding the obstruction event if the other sensitivity levels had been used. In other words, if controller 70 determines that a potential collision event is a true collision event, controller 70 is configured to subsequently determine if using each of the other sensitivity levels (see FIGS. 11 and 12) of the obstruction detection system would have changed its conclusion to a false collision event. On the other hand, if controller 70 determines that a potential collision event is a false collision event, it is configured to determine if using each of the other sensitivity levels would have changed its conclusion to a true collision event. From these analyses, controller 70 is configured, in at least some embodiments, to provide suggestions to the user to use a different sensitivity level.

In some embodiments, if controller 70 determines that more than X number of potential collision events turn out to be false collision events, yet those false collision events could have been avoided by using a different sensitivity level, controller 70 is configured to automatically provide a suggestion to the user to use a different sensitivity level for the obstruction detection system. Alternatively, or additionally, if the percentage of the false collision events relative to the total potential collision events exceeds a certain threshold, controller 70 may be configured to automatically suggest using a different sensitivity level. As yet another alternative, controller 70 may be configured to automatically suggest a different sensitivity level to the user whenever the user activates the manual override control and/or in response to still other triggers. Such suggestions for a different sensitivity threshold are, in at least one embodiment, only provided by controller 70 if controller 70 has determined that using a different sensitivity threshold would have avoided a false collision event.

When making suggestions of a different sensitivity level to the user, controller 70 may display a message on display 60 along the lines of “Your bed has been utilizing obstruction detection with the High sensitivity level. Recent data suggests that you could improve its performance by changing to the Medium or Low sensitivity levels. Would you like to make this change? (Y/N)” Of course, still other types of messages may be displayed to the user.

In some embodiments, controller 70 may also, or additionally, record the location of each obstruction detection event and, if obstructions are repetitively detected in the same area, controller 70 may automatically display a message to the user recommending that they check a particular area of the patient support apparatus 20 for obstructions before movement starts. Alternatively, or additionally, controller 70 may automatically display a message to the user to check for damage in a particular area of patient support apparatus 20 if repeated collisions are detected in the same area, and/or if weight changes (or absolute weight readings) exceed one or more thresholds.

In some embodiments, controller 70 is configured to analyze the log of potential obstruction detection events (and/or the subset of potential obstruction detection events that are true) to provide reminders to the caregivers to check for obstructions before they activate a movement control. For example, if more than a threshold number of obstruction detection events occur within a given time period, controller 70 may be configured to occasionally and/or repeatedly provide reminders to a user to check for obstructions in response to the user activating a movement control. In such situations, controller 70 may delay driving one or more of the actuators 26 and/or 66 until the user acknowledges that they have checked for obstructions. After they provide the acknowledgement, controller 70 will then begin driving the corresponding actuator(s) 26 and/or 66 in response to the user activating a particular movement control.

In still other embodiments, controller 70 is configured to send the log of potential obstruction events, including the data collected during the potential obstruction detection event to a remote server, such as server 82, for the server to analyze using machine learning and/or artificial intelligence. The server performs theses artificial intelligence and/or machine learning techniques to better improve the accuracy of the obstruction detection algorithm 100. In other words, the server may be programmed to increase the ability of the algorithm 100 to differentiate between changes in weight that are due to weight being added to, or removed from, patient support apparatus 20 and changes in weight that are due to a component of patient support apparatus 20 moving into an obstruction. The results of the analysis may be sent back to the patient support apparatus 20, and controller 70 may utilize the results to update and/or modify algorithm 100. The changes to algorithm 100 may include modifications to any feature of the algorithm, as well as modifications to the sensitivity levels and/or the numeric values used for the different sensitivity levels.

In some embodiments, controller 70 is configured to log every potential obstruction detection event for every one of the sensitivity levels that are possible, rather than for just the sensitivity level that is currently selected. For example, if the obstruction detection system has three sensitivity levels that correspond to second thresholds (used at step 112) of thirty, sixty, and ninety kilograms, controller 70 will log data each time a change in weight is detected at step 112 that exceeds thirty kilograms, each time a change in weight is detected at step 112 that exceeds sixty kilograms, and each time a change in weight is detected at step 112 that exceeds ninety kilograms, regardless of which one of the second thresholds is currently being used. In this manner, controller 70 is able to log data that enables it to determine if a different sensitivity level would have resulted in the obstruction detection system detecting an obstruction and, if so, whether the detection would have been a true or false obstruction detection. This data can then be used to make the recommendation(s) discussed above. When logging every potential obstruction event for all of the different sensitivity levels, controller 70 may continue to log the maximum and residual weight changes, even in those instances where motion is not stopped (due to the currently active second threshold not being exceeded at step 112 and/or due to a rate analysis that indicates an actual obstruction was not detected, and/or due to other reasons). In such instances, controller 70 may record the maximum weight change as the maximum weight change that occurs at any point during a predetermined amount of time after the second threshold is breached, and may record the residual weight change as the weight change at the moment that controller 70 would otherwise have ceased driving the actuator(s) 26 and/or 66 (or a predetermined time period after).

For example, suppose the current second threshold of step 112 is set to sixty kilograms and a weight change is detected that exceeds thirty kilograms (the high sensitivity level), but that is less than sixty kilograms. Because the weight change is less than sixty kilogram, controller 70 will not stop motion. However, because the weight change exceeded thirty kilograms (the high sensitivity level), controller 70 will log a maximum weight change, a residual weight change, and/or other data. In some embodiments, controller 70 (or an offboard server) will analyze this recorded data to determine if change in weight of more than thirty kilograms corresponded to an actual obstruction, or if it was a false obstruction. This true/false determination can then be used to recommend a more accurate sensitivity level, as discussed above.

In some embodiments, controller 70 may also be configured to determine and record the peak weight readings (positive or negative) in addition to the other data discussed herein. Such peak weight readings refer to the maximum and minimum weight readings detected, rather than, say, the maximum weight change. In other words, instead of just monitoring data regarding the changes in weight that are detected, controller 70 may be configured to also monitor the weight reading values themselves. The monitoring of the peak values allows controller 70 to detect abuse cases where patient support apparatus 20 is being driven in a manner where the forces (weight) applied to it exceed the maximum safe operating limits of the patient support apparatus. In such embodiments, controller 70 may be configured to automatically stop motion whenever a peak force reading exceeds a safety limit. This automatic stopping of motion due to a peak force exceeding a threshold may be carried out separately from algorithm 100, or algorithm 100 may be modified to include this monitoring of peak forces.

Still other changes to algorithm 100 may be made beyond those previously mentioned. For example, in some embodiments, algorithm 100 may be modified such that, either in addition to, or in lieu of, the determination at step 114 (FIG. 8) of whether the change in weight is within an ingress/egress zone 160, controller 70 may be configured to compare the location of the change in weight to a location of the patient immediately prior to the change in weight detected at step 112. If the change in weight is positioned within a threshold distance of the patient's location, controller 70 concludes that the change in weight is due to the patient shifting more of his or her weight off the floor and onto patient support apparatus 20, or vice versa, in which case the change in weight is not due to an obstruction, but rather due to the shift in the patient's weight. In some embodiments, the threshold distance may be in the range of about zero to one meter, although other threshold distances may be used.

In some embodiments, controller 70 may be configured to automatically determine which motion controls on control panels 58a-c will move patient support apparatus 20 away from a detected obstruction and which motion controls will cause patient support apparatus 20 to move further toward the obstruction. In such embodiments, controller 70 may be configured to temporarily disable the movement controls that will cause patient support apparatus 20 to move further towards the obstruction, but continue to enable all of the movement controls that will cause patient support apparatus 20 to move away from the obstruction. This temporary disabling of the set of movement controls that will cause patient support apparatus 20 to move further toward the obstruction may be implemented, in some embodiments, on a patient support apparatus 20 that includes no recovery control 206. Alternatively, patient support apparatus 20 may include a recovery control 206, but instead of disabling all of the movement controls on control panels 56a-c, as discussed previously, controller 70 may be configured to only temporarily disable those controls that will cause patient support apparatus 20 to move further into an obstruction. In such embodiments, the controls 58 that are not temporarily disabled may include only the control(s) 58 whose function is temporarily reassigned to carry out the recovery control 206, and/or it may include one or more other controls 58 whose functions are not temporarily reassigned.

Controller 70 is configured to determine which movement controls will cause patient support apparatus 20 to move further into an obstruction by taking into account the location of the obstruction on patient support apparatus 20, as well as the sign of the weight change. Obstructions that are hit while a component of patient support apparatus 20 is moving upward will cause a positive weight change (an increase in weight), while obstructions that are hit while a component of patient support apparatus 20 is moving downward will cause a negative weight change (a decrease in weight). By using this information, along with center of gravity of the detected weight change and the knowledge of which actuator(s) 26 and/or 66 were being driven (and in what direction) when the obstruction was impacted, controller 70 can determine which movement of actuator(s) 26 and/or 66 will cause further pressure to be exerted against the obstruction, and which movement of actuator(s) 26 and/or 66 will relieve the pressure exerted on the obstruction. Controller 70 then temporarily disables those movement controls that will increase this pressure while keeping those movement controls enables that will relieve this pressure. The temporary disabling of the controls may persist until one of the non-disabled controls is used to back the patient support apparatus 20 away from the obstruction and/or until the recovery control 206 (if present) is utilized. In some embodiments, controller 70 flashes one or more of the controls on the siderail controls panels 56b and/or 56c to indicate that a potential obstruction has been detected, and to give the user visual feedback acknowledging that controller 70 has deliberately stopped driving the one or more actuators 26 and/or 66.

In some embodiments, algorithm 100 may be modified from what is shown in FIG. 8 such that controller 70 continues to drive the actuator(s) 26 and/or 66, even if a change in weight greater than the second threshold is detected at step 112, provided one or more criteria are met. The criteria include an analysis of the direction of movement of whatever component or components are being driven by actuator(s) 26 and/or 66, whether the change in weight detected at step 112 is positive or negative, and, in some instances, the location of the potential obstruction. Using this information controller 70 may be configured to dismiss certain changes in weight that exceed the second threshold of step 112 as not being due to obstructions. For example, if both lifts 26 and/or actuator 66a and/or 66b of patient support apparatus 20 are being driven downward (toward the floor) and an increase in weight over the second threshold of step 112 is detected at any location on patient support apparatus 20, controller 70 may be configured to not stop movement of patient support apparatus 20, but instead proceed to step 104 and re-determine a baseline weight (or take other action). This is because, when a component of patient support apparatus 20 is moving downward toward a floor, any obstruction it will hit will cause a decrease in weight on the patient support apparatus 20, rather than in increase in weight. Consequently, if an increase in weight is detected during downward movement, it must be due to the addition of a weight on patient support apparatus 20, rather than due to an obstruction.

As another example, if both lifts 26 and/or actuator 66a and/or 66b are being driven upward (toward the ceiling) and a decrease in weight over the second threshold of step 112 is detected, controller 70 may be configured to not stop movement of patient support apparatus 20, but instead proceed to step 104 and re-determine a baseline weight (or take other action). This is because, when a component of patient support apparatus 20 is moving upward, any obstruction will cause an increase in weight on the patient support apparatus 20. Consequently, if a decrease in weight id detected during upward movement, it must be due to the removal of a weight on patient support apparatus 20, rather than due to an obstruction.

As yet another example, if only a single lift 26 is being lowered and an increase in weight greater than the second threshold (step 112) is detected at the same end of the patient support apparatus 20, controller 70 may automatically conclude this is not due to an obstruction, and therefore re-determine a baseline weight and/or take other action. However, if only a single lift 26 is being lowered and an increase in weight greater than the second threshold is detected at the opposite end of the patient support apparatus 20, this may be due to an obstruction (see the example shown in FIG. 27), and controller 70 may therefore continue to perform algorithm 100 in the manner illustrated in FIG. 8.

In some embodiments, rather than allowing the user to select a sensitivity level for the obstruction detection system that is qualitative (e.g. low, medium, or high), controller 70 may be configured to allow the user to enter a numeric value for second threshold used at step 112 (and/or for the third threshold(s) used at step 116). Alternatively, or additionally, controller 70 may be configured to allow the user to select a percentage value for the value of the second threshold used at step 112, such as a percentage of the patient's weight, a percentage of the empty weight of patient support apparatus 20 when no patient is present, and/or a percentage of another weight.

Algorithm 100 may also be modified to perform differently after an obstruction has been detected. For example, if an additional 100 kg weight from an obstruction is detected by the obstruction detection system and movement has been automatically stopped, if the user presses on the recovery control 206, or the user activates a non-disabled movement control to back the component away from the obstruction, controller 70 will detect a 100 kg drop in the weight from the obstruction no longer pressing against the patient support apparatus 20. In order to prevent controller 70 from concluding that this 100 kg drop in weight is due to another obstruction, controller 70 may be configured to temporarily suspend algorithm 100 when recovery control 206 is activated and/or to temporarily suspend algorithm 100 when controller 70 is driving actuator(s) 26 and/or 66 away from an obstruction. Alternatively, or additionally, instead of suspending algorithm 100 completely, controller 70 may continue to execute algorithm 100 but ignore any 100 kg decrease in weight that occurs in substantially the same place as the previously 100 kg increase in weight, or that occurs within a short time period (e.g. within a few seconds) after that 100 kg increase was detected. These changes help prevent controller 70 from concluding that the “relief” from hitting an obstruction has resulted in yet another obstruction being impacted.

In some embodiments, whenever controller 70 concludes that an obstruction has been detected, controller 70 may send a message to server 82 via network transceiver 68 and a wireless access point 80. Server 82, which may be executing a caregiver assistance application 84 may then send a notification to one or more portable electronic devices 86 indicating that an obstruction has been hit by the patient support apparatus 20. In some embodiments, the caregiver assistance application 84 may also send additional information about the obstruction impact to the one or more portable electronic devices 86, such as the location of the impact, the change in weight detected, whether the obstruction has been resolved, and/or other information.

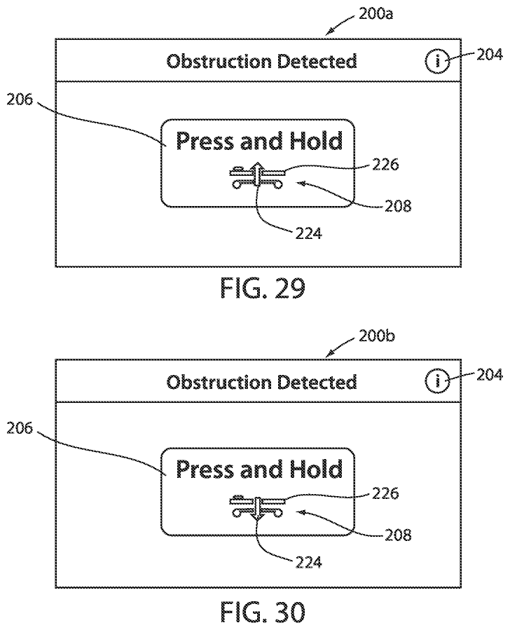

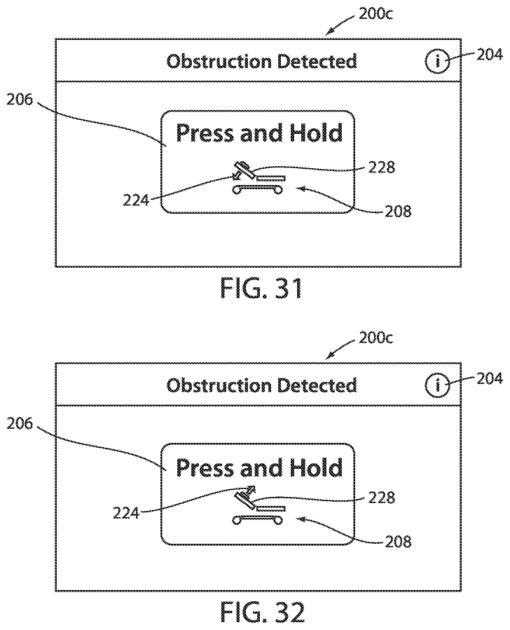

In some embodiments, controller 70 may be configured to display the warning screens 200a-d of FIGS. 29-32 instead of the warning screen 200 of FIG. 23. In other words, in some embodiments, controller 70 is configured to display one of screens 200a-d when controller 70 determines that an obstruction was hit during movement of one or more components of patient support apparatus 20. Warning screens 200a-d include the information icon 204 and a recovery control 206 similar to screen 200 of FIG. 23. However, screens 200a-d of FIGS. 29-32, unlike screen 200, includes a patient support apparatus icon 208 on the recovery control 206. The patient support apparatus icon 208 includes an arrow 224 as well as a litter frame graphic 226 (FIGS. 29 & 30) or a Fowler section graphic 228 (FIGS. 31 & 32). Controller 70 displays arrow 224 in a manner that indicates which direction the litter frame 28 or Fowler section 42 needs to move in order to move away from the obstruction.

In other words, if controller 70 determines that an obstruction was hit on the underside of litter frame 28 while litter frame 28 was being lowered (one or both of lifts 26 were being lowered), controller 70 is, in some embodiments, configured to display screen 200a of FIG. 29, which includes arrow 224 positioned next to litter frame graphic 226 and pointing upward. The upward arrow 224 of FIG. 29 indicates that litter frame 28 needs to be moved upward (raised) in order to move away from the obstruction. If controller 70 determines that an obstruction was hit on the top side of litter frame 28 while litter frame 28 was being raised (one or both of lifts 26 were being raised), controller 70, in some embodiments, is configured to display screen 200b of FIG. 30, which includes arrow 224 positioned next to litter frame graphic 226 and pointing downward. The downward arrow 224 of FIG. 30 indicates that litter frame 28 needs to be lowered in order to move away from the obstruction.

If controller 70 determines that an obstruction was hit while Fowler section 42 was being raised (Fowler actuator 66b was driving Fowler section 42 upward), controller 70 is, in some embodiments, configured to display screen 200c of FIG. 31, which includes arrow 224 positioned next to Fowler graphic 228 and pointing downward. The downward arrow 224 of FIG. 31 indicates that Fowler section 42 needs to be lowered in order to move away from the obstruction. If controller 70 determines that an obstruction was hit while Fowler section 42 was being lowered (Fowler actuator 66b was driving Fowler section 42 downward), controller 70 is, in some embodiments, configured to display screen 200d of FIG. 32. The upward arrow 224 of FIG. 32 indicates that Fowler section 42 needs to be raised in order to move away from the obstruction. In some embodiments, in addition to arrow 224 and/or in lieu of arrow 224, controller 70 may be configured to display an indicator showing where an obstruction has impacted patient support apparatus 20. The indicator may be displayed with respect to patient support apparatus icon 208 or with respect to some other graphical representation of patient support apparatus 20.

As was noted previously, the force sensors 54 used by the obstruction detection system may be the same force sensors 54 that are used to implement an exit detection system and/or a scale system onboard patient support apparatus 20. In some embodiments, when force sensors 54 are used for both the obstruction detection system and either or both of an exit detection system and/or a scale system, controller 70 may be configured to utilize outputs from the force sensors 54 for the obstruction detection system that are filtered at different time intervals than those that are used for exit detection system and/or scale system. For example, in some embodiments, outputs from the force sensors 54 may be read every eight milliseconds or so, and the obstruction detection system may utilize a 96 millisecond moving average of those readings for carrying out algorithm 100, while controller 70 (or another controller) may utilize a longer moving average of those readings when carrying out the exit detection function and/or scale function. The longer moving average may be on the order of 496 milliseconds. It will be understood, of course, that readings may be taken from force sensors at rates other than every eight milliseconds, that a moving average other than 96 milliseconds may be used for the obstruction detection system, and/or that a moving average other than 496 milliseconds may be used for the scale system and/or the exit detection system. It will also be understood that filters other than a moving average may be applied to the readings from the force sensors 54.

In those embodiments of patient support apparatus 20 that include a scale system, controller 70 may be configured to use the outputs from force sensors 54 to implement the scale system in a manner that include any of the features or functions of the scale systems disclosed in the following commonly assigned U.S. patent references: U.S. patent application Ser. No. 63/255,211 filed Oct. 13, 2021, by inventors Sujay Sukumaran et al, and entitled PATIENT SUPPORT APPARATUS WITH AUTOMATIC SCALE FUNCTIONALITY; U.S. Pat. No. 10,357,185 issued to Marko Kostic et al. on Jul. 23, 2019, and entitled PERSON SUPPORT APPARATUSES WITH MOTION MONITORING; U.S. Pat. No. 11,033,233 issued to Michael Hayes et al. on Jun. 15, 2021, and entitled PATIENT SUPPORT APPARATUS WITH PATIENT INFORMATION SENSORS; U.S. patent application Ser. No. 16/992,515 filed Aug. 13, 2020, by inventors Kurosh Nahavandi et al, and entitled PATIENT SUPPORT APPARATUS WITH EQUIPMENT WEIGHT LOG; and U.S. patent application Ser. No. 63/255,223, filed Oct. 13, 2021, by inventors Sujay Sukumaran et al, and entitled PATIENT SUPPORT APPARATUS WITH PATIENT WEIGHT MONITORING, the complete disclosures of all of which are incorporated herein by reference.

When controller 70 uses the outputs of force sensors 54 to carry out an exit detection function, controller 70 may be configured to implement any of the features, functions, and/or components of any of the exit detection systems disclosed in any of the following commonly assigned U.S. patent references: U.S. patent application Ser. No. 63/245,279 filed Sep. 17, 2021, by inventors Jerald Trepanier et al, and entitled PATIENT SUPPORT APPARATUSES WITH PATIENT MONITORING; U.S. patent application Ser. No. 17/318,476 filed May 12, 2021, by inventors Sujay Sukumaran et al, and entitled PATIENT SUPPORT APPARATUS WITH AUTOMATIC EXIT DETECTION MODES OF OPERATION; U.S. patent application Ser. No. 16/917,004 filed Jun. 30, 2020, by inventors Sujay Sukumaran et al, and entitled PERSON SUPPORT APPARATUS WITH ADJUSTABLE EXIT DETECTION ZONES; and U.S. patent application Ser. No. 15/266,575 filed Sep. 15, 2016, by inventors Anuj K. Sidhu et al, and entitled PERSON SUPPORT APPARATUSES WITH EXIT DETECTION SYSTEMS, the complete disclosures of all of which are incorporated herein by reference.

It will be understood that the concepts disclosed herein may all be combined together in a patient support apparatus 20, or they may be individually integrated into a patient support apparatus 20. For example, in some embodiments, patient support apparatus 20 includes an obstruction detection system that implements algorithm 100 in the manner shown in FIG. 8, but that does not use a recovery control 206 and/or that does not analyze the rate at which the change in weight occurs. Alternatively, in some embodiments, patient support apparatus 20 includes a recovery control 206, but does not utilize algorithm 100 (and/or in some embodiments does not utilize force sensors 54 at all for obstruction detection, but instead may use pressure switches or other direct contact sensors). As yet another alternative, in some embodiments, controller 70 does not determine the location where an obstruction impacts patient support apparatus 20 (i.e. it does not calculate a center of gravity of the weight change at step 114 of algorithm 100), it does not utilize different sensitivity levels, but it does display the screens of FIGS. 23-27 (or a variation thereof). In still other embodiments, any other combination of the individual screens, functions, and/or features of the obstruction detection system disclosed herein may be integrated into a patient support apparatus 20.

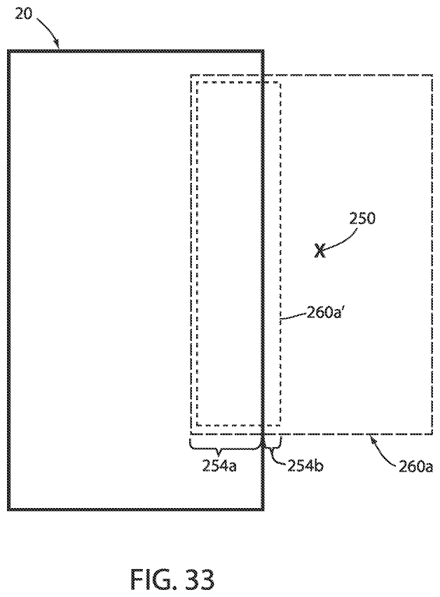

FIG. 33 illustrates an embodiment of patient support apparatus 20 that may implement a modified version of obstruction detection algorithm 100. More specifically, FIG. 33 shows a patient support apparatus 20 as a rectangular shape generally corresponding to the footprint of patient support apparatus 20 as viewed from above. In other words, the rectangular shape of patient support apparatus 20 in FIG. 33 generally represents the perimeter of patient support apparatus 20 when viewed from above patient support apparatus. FIG. 33 also illustrates a dynamic tag zone 260a that is defined around a tag 250. Tag 250 is adapted to be worn by a person, such as a patient, a caregiver, a visitor, and/or another type of person expected to be present in a healthcare facility. Controller 70, in the embodiment shown in FIGS. 33-36, is configured to communicate with one or more tag sensor(s) 252 positioned onboard patient support apparatus 20 (FIG. 5) and tag sensor(s) 252 is/are configured to detect the position of tag 250 with respect to patient support apparatus 20. In such embodiments, tag 250 may be an RFID tag and each tag sensor 252 may be an RFID sensor adapted to detect the position of tag 250 with respect to patient support apparatus 20.

In some embodiments, each tag 250 is an ultra-wideband tag and each tag sensor 252 is an ultrawideband transceiver adapted to detect the distance between, and/or the angular relationship between, itself and tag 250. The position of each tag sensor 252 onboard patient support apparatus 20 is stored in memory 72 and used by controller 70 to determine the position of tag 250 relative to the boundary, or footprint, of patient support apparatus 20. In those embodiments where tag 250 is an ultra-wideband tag and sensor(s) 252 is/are ultra-wideband transceivers, tag sensor(s) 252 and tags 250 may be the same as the UWB transceivers and tags disclosed in any of the following list of commonly assigned patent applications; controller 70 may be configured to perform any of the same functions as the controllers disclosed in any of the following list of commonly assigned patent applications; and/or patient support apparatus 20 may be configured to include any of the structures, functionality, and/or algorithms of the patient support apparatuses disclosed in the disclosed in any of the following list of commonly assigned patent applications: U.S. patent application Ser. No. 17/559,339 filed Dec. 22, 2021, by inventors Alexander Bodurka et al, and entitled PATIENT SUPPORT APPARATUS AND MEDICAL DEVICE NETWORKS; U.S. patent application Ser. No. 18/028,590 filed Mar. 27, 2023, by inventors Celso Pereira et al, and entitled SYSTEM FOR DETERMINING PATIENT SUPPORT APPARATUS AND MEDICAL DEVICE LOCATION; U.S. patent application Ser. No. 17/695,151 filed Mar. 15, 2022, by inventors Krishna Bhimavarapu et al, and entitled EXERCISE DEVICE AND PATIENT SUPPORT APPARATUS; PCT patent application PCT/US2022/031012 filed May 26, 2022, by inventors Thomas Deeds et al, and entitled SYSTEM FOR ASSOCIATING DEVICE DATA; PCT patent application PCT/US2022/043585 filed Sep. 15, 2022, by inventors Kirby Neihouser et al, and entitled SYSTEM FOR LOCATING PATIENT SUPPORT APPARATUSES; PCT application PCT/US2022/043586 filed Sep. 15, 2022, by inventors Jerald Trepanier et al, and entitled PATIENT SUPPORT APPARATUS WITH PATIENT MONITORING; PCT application PCT/US2022/043587 filed Sep. 15, 2022, by inventors Madhu Sandeep Thota et al, and entitled PATIENT SUPPORT APPARATUS COMMUNICATION AND LOCATION SYSTEM; PCT application PCT/US2023/012173 filed Feb. 2, 2023, by inventors Madhu Sandeep Thota et al, and entitled COMMUNICATION SYSTEM FOR PATIENT SUPPORT APPARATUSES; PCT application PCT/US2023/013722 filed Feb. 23, 2023, by inventors Jerald Trepanier et al, and entitled COMMUNICATION SYSTEM FOR PATIENT SUPPORT APPARATUSES AND TEMPERATURE MANAGEMENT DEVICES; PCT application PCT/US2023/024644 filed Jun. 7, 2023, by inventors Jerald Trepanier et al, and entitled COMMUNICATION SYSTEM FOR PATIENT SUPPORT APPARATUSES; PCT application serial number PCT/US2023/026462 filed Jun. 28, 2023, by inventors Krishna Bhimavarapu et al, and entitled BADGE AND PATIENT SUPPORT APPARATUS COMMUNICATION SYSTEM; PCT application PCT/US2023/026440 filed Jun. 28, 2023, by inventors Jerald Trepanier et al, and entitled BADGE AND PATIENT SUPPORT APPARATUS COMMUNICATION SYSTEM; PCT application PCT/US2023/026418 filed Jun. 28, 2023, by inventors Madhu Sandeep Thota et al, and entitled BADGE AND PATIENT SUPPORT APPARATUS COMMUNICATION SYSTEM; U.S. provisional patent application Ser. No. 63/428,075 filed Nov. 27, 2022, by inventors Kirby Neihouser et al, and entitled COMMUNICATION TOOL FOR UWB-EQUIPPED PATIENT DEVICES; and/or U.S. provisional patent application Ser. No. 63/428,077 filed Nov. 27, 2022, by inventors Madhu Sandeep Thota et al, and entitled PATIENT SUPPORT APPARATUS COMMUNICATION SYSTEM, the complete disclosures of which are all incorporated herein in their entirety.

In the embodiment shown in FIG. 33, controller 70 is configured to implement a modified version of algorithm 100 that either replaces or supplements step 114 with a different step. In that different step, controller 70 is configured to compare the center of gravity of the weight change to the dynamic tag zone 260a. If the center of gravity of the weight change is inside of the dynamic tag zone 260a, controller 70 is configured to move to step 116, or a step similar to step 116 that may utilize different weight thresholds (e.g. is ΔW between something other than −90 kg and 120 kg). If the center of gravity of the weight change is outside of the dynamic tag zone 260a, controller 70 is configured to move to step 124, or a step similar to step 124, to see if the detected change in weight (that exceeded the first threshold of step 112) is actually due to an obstruction being encountered (via steps 124-126, or a variation of these steps).

The result of the aforementioned modifications to algorithm 100 is that, greater weight changes are required to occur within dynamic tag zone 260a to trigger an obstruction detection than are required outside of dynamic tag zone 260a to trigger an obstruction detection. This is because the tag 250 is worn by a person who may place his or her arm on patient support apparatus 20, or otherwise lean on patient support apparatus 20, and thereby cause a weight change that is not due to an obstruction, but rather is due to the person exerting a force on patient support apparatus 20. Accordingly, in order to counter these weight changes that might otherwise lead to false detections of obstructions, controller 70 uses a higher weight threshold for detecting an obstruction when the weight change occurs within tag zone 260a.

As was noted above, in some embodiments, the comparison of the location of the change in weight to dynamic tag zone 260a replaces step 114 in algorithm 100. In such instances, controller 70 does not look to see if the detected weight change has occurred inside or outside of an ingress/egress zone 160, but instead looks to see if the detected weight change has occurred inside or outside of the dynamic tag zone 260a. Depending upon the results of this analysis, controller 70 proceeds to step 116 and/or step 124 any may follow any of the subsequent steps, or may implement any of the modifications discussed above with respect to the step(s) subsequent to step 114 of algorithm 100.

In some embodiments, controller 70 may be configured to implement both step 114 and the above-described step in which controller 70 compares the center of gravity of the change in weight to dynamic tag zone 260a. Such embodiments may be particularly useful in healthcare facilities where not all caregivers and/or patients are expected to be wearing tags 250. In such embodiments, controller compares the location (i.e. center of gravity) of the obstruction to both the ingress/egress zone 260a and, if present, dynamic tag zone 260a. If the location of the weight change is in either of these zones 160 and/or 260a, controller 70 uses a larger weight threshold for concluding an obstruction was hit during a movement of a component of patient support apparatus 20. In other words, controller 70 requires a larger force to be detected before stopping movement of the component if the force is detected in either or both of these zones 106 and/or 260a.

In some embodiments, controller 70 may be configured to use dynamic tag zone 260a if a tag 250 is detected within the vicinity of patient support apparatus 20 (e.g. zone 260a overlaps with a portion of the footprint of patient support apparatus 20), and, if no tag 250 is detected within the vicinity, to use ingress/egress zone 160, but to never compare the location of the change in weight to both dynamic tag zone 260a and ingress/egress zone 160. In such embodiments, controller 70 only compares the position of the change in weight detected at step 112 to either, but not both, of zones 160 or 260a.

In still other embodiments, patient support apparatus 20 may be configured to not detect UWB tags 250 and/or to not utilize dynamic tag zone 260a when performing algorithm 100 (or a modified version of it). Alternatively, in some embodiments, patient support apparatus 20 may be configured to only use dynamic tag zone 260a, and not ingress/egress zone 160, when performing a modified version of algorithm 100.

In some embodiments, dynamic tag zone 260a is defined as a fixed square, rectangle, circle, or other shape that has the location of tag 250 generally defined in the center of tag zone 260a. The dynamic tag zone 260a is considered dynamic because it moves as the person who is wearing the tag 250 moves. The location of tag zone 260a is therefore dynamic, rather than fixed. In some embodiments, dynamic tag zone 260a may be square shaped with approximately 1-2 meter sides, although other dimensions may be utilized. In general, dynamic tag zone 260a may be dimensioned to extend from the tag approximately the length of a typical person's arms, thereby ensuring that if the person places his/her hand or arm on patient support apparatus 20, the change in weight detected will be located within the dynamic tag zone 260a (and therefore be less likely to trigger a false obstruction detection event).

In some embodiments, controller 70 is configured to determine if the change in weight detected at step 112 (assuming it is greater than the first threshold) is located anywhere within dynamic tag zone 260a. In other embodiment, controller 70 is configured to determine if the change in weight detected at step 112 (assuming it is greater than the first threshold) is located anywhere within a reduced dynamic tag zone 260a′. One example of the reduced dynamic tag zone 260a′ is shown in FIG. 33. Reduced dynamic tag zone 260a′ is defined by a first portion 254a of dynamic tag zone 260a that overlaps with patient support apparatus 20, as well as a second portion 254b of dynamic tag zone 260a that extends outwardly from the perimeter of patient support apparatus 20 a fixed amount. In some embodiments, the fixed amount may be on the order of ten to twenty centimeters, although other distances may be used.

As was described above, in the embodiment shown in FIG. 33, controller 70 is configured to repetitively determine the position of tag 250 with respect to patient support apparatus 20 (using sensor(s) 252) and to repetitively update the position of dynamic tag zone 260a (and, if applicable, the position of first and second portions 254a and 254b). In this manner, as the wearer of tag 250 moves, the location of dynamic tag zone 260a also moves. Controller 70 uses the location of dynamic tag zone 260a at the time a change in weight is detected that exceeds the first threshold at step 112.

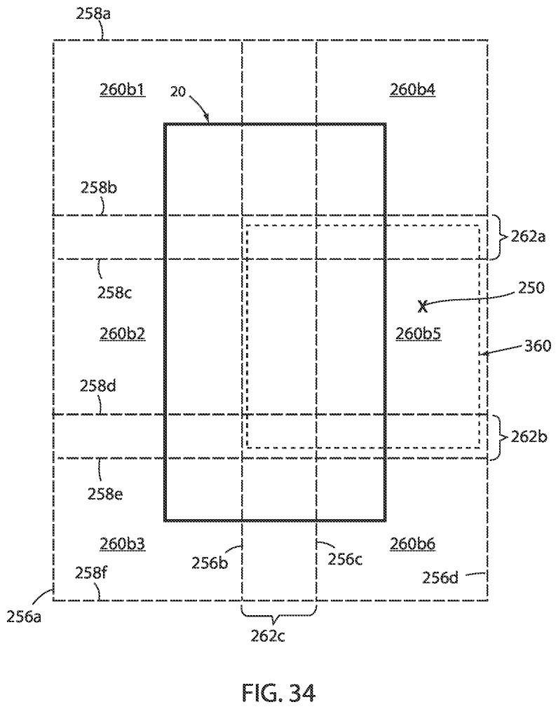

FIG. 34 illustrates an example of a patient support apparatus 20 that is configured to select one or more static tag zones 260b instead of a dynamic tag zone 260a. Static tag zones 260b differ from dynamic tag zone 260a in that static tag zones 260b are defined during the manufacture of patient support apparatus 20 (or at some other point in time prior to implementation of algorithm 100). Static tag zones 260b also differ from dynamic tag zones 260a in that there are multiple static tag zones 260b for a single tag 250 (or for more tags 250), while there is only one dynamic tag zone 260a for each tag 250. In the embodiment shown in FIG. 34, there are six different static tag zones 260b: zone 260b1, 260b2, 260b3, 260b4, 260b5, and 260b6. Zone 260b1 is defined by a top boundary line 258a, a bottom boundary line 258c, a left boundary line 256a, and a right boundary line 256c. Zone 260b2 is defined by a top boundary line 258b, a bottom boundary line 258e, a left boundary line 256c, and a right boundary line 256b. Zone 260b3 is defined by a top boundary line 258d, a bottom boundary line 258f, a left boundary line 256a, and a right boundary line 256c. Zone 260b4 is defined by a top boundary line 258a, a bottom boundary line 258c, a left boundary line 256b and a right boundary line 256d. Zone 260b5 is defined by a top boundary line 258 b, a bottom boundary line 258e, a left boundary line 256b, and a right boundary line 256d. Zone 260b6 is defined by a top boundary line 258d, a bottom boundary line 258f, a left boundary line 256b, and a right boundary line 256d.

In the example shown in FIG. 34, controller 70 uses tag sensor 252 to determine that tag 250 is currently positioned in static tag zone 260b5. As a result, controller 70 compares any change of weight greater than the first threshold (detected at step 112 of algorithm 100) to static tag zone 260b5. If the change in weight occurred within static tag zone 260b5, controller 70 uses a modified threshold (from the first threshold of step 112), such as the second threshold of step 116, or another modified threshold. If the change in weight is greater than this modified threshold, controller 70 stops movement of the moving component of patient support apparatus 20 and either immediately issues an obstruction alert or performs step 120 and, if the conditions of step 120 are met, then issues an obstruction alert.

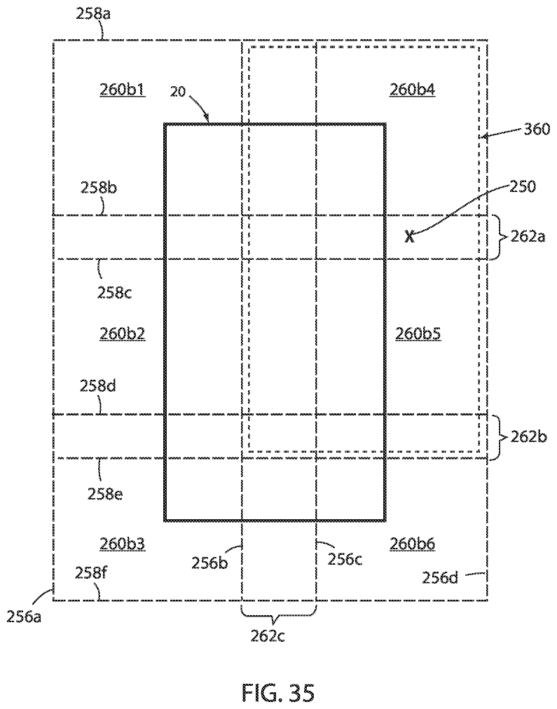

As was noted above, the static tag zones 260b do not move. Instead, they are all defined in fixed relationship to patient support apparatus 20. Thus, if tag 250 moves to different locations within zone 260b5, but doesn't move outside of zone 260b5, controller 70 continues to select zone 260b5 as the active zone 360 (with one exception discussed below with respect to FIG. 35). If tag 250 moves outside of zone 260b5 to another static zone, such as zone 260b6, controller 70 switches to selecting zone 260b6 as the active zone 360. And, of course, if tag 250 moves outside of any of zones 260b1-b6, controller 70 selects no active zone 360 and implements algorithm 100 without any of the modifications discussed herein relating to tag 250 (but which may include any of the non-tag related modifications discussed herein). The term “active zone” refers to the zone to which controller 70 compares the change in weight detected at step 112.

As noted, there is one exception to when controller 70 may change the active zone in response to movement of tag 250 within a particular zone 260b. This exception relates to the overlap areas 262a, 262b, and 262c. Overlap areas 262 define areas where two or more static tag zones 260b overlap with each other. Overlap area 262a is comprised of a portion in which zones 260b4 and 260b5 overlap with each other, as well as another portion where zones 260b1 and 260b2 overlap with each other. There is also a central portion of overlap area 262a where all four zones 260b1, 260b2, 260b4, and 260b5 overlap with each other. Overlap areas 262b and 262c also have similar portions where different ones of zones 260b1-260b6 overlap with each other. If a tag 250 moves into an overlap area 262, controller 70 is configured, in some embodiments, to select all of the zones 260b that overlap the particular portion of the overlap area 262 in which the tag 250 is located. An example of this is shown in FIG. 35.

In FIG. 35, tag 250 has moved into overlap area 262a. Specifically, tag 250 has moved into a portion of overlap area 262a in which zone 260b4 and 260b5 overlap. Accordingly, controller 70 selects both zones 260b4 and 260b5 as the active zone 360. Consequently, if controller 70 detects a change in weight at step 112 that exceed the first threshold, and the change in weight occurs in either zones 260b4 or 260b5, then controller 70 is configured to compare the change in weight to a second threshold, as discussed above, as part of its algorithm for determining if an obstruction was impacted.

It will be understood that, although the dynamic tag zones 260a and static tag zones 260b have been illustrated in FIGS. 33-35 as two dimensional rectangles or squares, zones 260a and/or 260b may be three-dimensional zones in some embodiments. That is, instead of defining two dimensional areas, zones 260a and/or 260b may define three-dimensional volumes. In some of such instances, controller 70 may take into account not only whether tag 250 is within a zone, but also its height within that zone. In such embodiments, controller 70 may utilize the height information to adjust one or more of the values of the first or second thresholds (steps 112 and/or 116), depending not only upon the direction of movement of the moving component, but also depending upon whether force sensors 54 detect an increased change in weight or a decreased change in weight.

For example, in some embodiments, if controller 70 detects (using sensor(s) 252) that a tag 250 is positioned at a height lower than the current height of litter frame 28, this may be indicative of a person bending over, crouching, or otherwise positioning themselves underneath a portion of patient support apparatus 20. In such situations, controller 70 may be configured to decrease the threshold used at step 116 (or a modified version thereof), thereby increasing the sensitivity of the obstruction detection within the corresponding zone 260a or b, and therefore helping to prevent litter frame 28, or another moving component, from being moved downward onto the person wearing the tag 250. In contrast, if the component is being moved upward, controller 70 may increase the threshold used at step 116 (or a modified version thereof), thereby decreasing the sensitivity of the obstruction detection system within that particular zone 260a or b. In contrast, if the tag is detected above the height of litter frame 28, it may be assumed that the person wearing the tag 250 is at little risk of being injured by an upward moving component of patient support apparatus 20 (and in some cases, little risk of being injured by a downward moving component of patient support apparatus 20). In such cases, controller 70 may increase the threshold for the corresponding zone 260a or 260b, thereby decreasing the sensitivity of the obstruction detection system.

In some embodiments, controller 70 may be configured to adjust one or more of the values used at step 116 (or a modified version thereof) depending upon whether tag 250 is positioned within the footprint of patient support apparatus 20 or positioned outside of the footprint of patient support apparatus 20. If the tag 250 is positioned outside of the footprint, there is a chance the person wearing the tag 250 may attempt to enter patient support apparatus 20, in which case a relatively large increase in weight is to be expected. On the other hand, if tag 250 is positioned inside of the footprint, there is a chance the person wearing the tag 250 may attempt to exit patient support apparatus 20, in which case a relatively large decrease in weight is to be expected. Controller 70 may therefore adjust the values of the threshold used at step 116 (or a modified version thereof), depending upon whether the tag is positioned inside or outside of the footprint of patient support apparatus 20. In general, controller 70 modifies the values such that there is a decreased sensitivity for increases in weight in the zone 260a or 260b corresponding to tag 250 if the tag 250 is positioned outside the footprint of patient support apparatus 20 (in which case an additional weight from the patient entering may be expected). Conversely, controller 70 may modify the values such that there is a decreased sensitivity for decreases in weight in the zone 260a or 260b corresponding to tag 250 if tag 250 is positioned inside the footprint of patient support apparatus 20 (in which case less weight due to the patient exiting may be expected).

Similarly, in some embodiments, controller 70 may modify the values used in step 116 (or a modified version thereof) depending upon whether the tag 250 belongs to a patient or a caregiver. In such embodiments, tag sensor(s) 252 are configured to receive identification data from tag 250, and controller 70 is configured to use that identification data to determine whether tag 250 is associated with a patient or a caregiver. Because caregivers are generally not expected to enter and exit patient support apparatus 20, while patients are, controller 70 may adjust the values used in algorithm 100 in different manners, depending upon whether a nearby tag 250 belong to a caregiver or a patient.

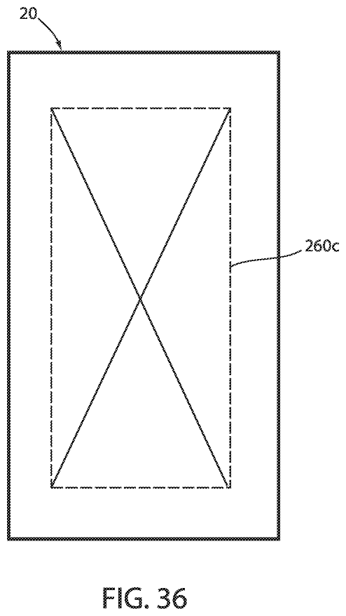

In some embodiments, controller 70 is configured to ignore any tag 250 that is detected within an exclusion zone 160c, such as the exclusion zone 160c shown in FIG. 36. In the embodiment shown in FIG. 36, exclusion zone 160c extends vertically (out of the page) a specified distance, such as approximately 20 to 50 centimeters, although other values may be used. When controller 70 detects tag 250 within exclusion zone 160c, regardless of whether or not tag 250 is also present in one or more zones 260a or 260b (or, in some embodiments, ingress/egress zone 160), controller 70 ignores the tag and carries out algorithm 100 without taking any steps that relate to tag 250. In other words, controller 70 implements algorithm 100 without making any adjustments to the first or second thresholds that are based on tag 250.

It will be understood that any of the concepts discussed with respect to FIGS. 33-36 may be carried out individually by themselves, or they may be combined with other concepts discussed in the other drawings herein. For example, one or more of the tag 250 concepts discussed with respect to any of FIGS. 33-36 may be implemented in a patient support apparatus 20 that uses a recovery control 206, or they may be implemented in a patient support apparatus 20 that does not include a recovery control 206. Similarly, in some embodiments, one or more of the tag 250 concepts discussed with respect to any of FIGS. 33-36 may be implemented in a patient support apparatus 20 that allows the user to select different sensitivity levels for the obstruction detection system, or they may be implemented in a patient support apparatus 20 that does not allow the user to select different sensitivity levels. In sum, any of the various features and functions of patient support apparatus 20 discussed herein may be implemented in a patient support apparatus 20 by themselves, or in any combination with one or more of the other features and functions.

In general, it can be seen from the foregoing description that the obstruction detection system described herein may utilize UWB to track the location of patients and/or caregivers. Using this location tracking, controller 70 can adjust the threshold for obstruction detection in a localized area to prepare for possible patient/caregiver interaction near that location. This allows for higher sensitivity to be used on the entire patient support apparatus 20, including the ingress/egress zone, while still limiting the number of false alarms caused by patient/caregiver interaction with patient support apparatus 20. This allows for better performance for obstruction detection, while further limiting false alarms. In some embodiments, the technology described herein may be implemented to varying degrees above and below litter frame 28. For instance, if a default obstruction detection threshold was chosen to be +20 kg/−20 kg (above/below frame 28), it is possible that, within a specified area around a patient or caregiver location, the threshold could be adjusted to +150 kg/−90 kg or even disabled above the frame 28, and 90 kg below the frame 28. Maintaining a threshold for below the frame 28 ensures that the base 22 of patient support apparatus 20 is not lifted off the ground by a potential true obstruction within the patient/caregiver zone. Additionally, it is more likely for a patient or caregiver to have their foot underneath patient support apparatus 20 than it would be for them to have an appendage caught above litter frame 28, so maintaining some threshold below the litter frame 28 is useful in that case.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1-31. (canceled)

32. A patient support apparatus comprising:
a support deck adapted to support a patient thereon;
a frame adapted to support the support deck;
a plurality of force sensors adapted to detect a weight supported by the frame;
an actuator adapted to move a component of the patient support apparatus;
a control panel including a movement control, wherein the movement control is adapted to drive the actuator when the movement control is activated; and
a controller adapted to detect if a change in the weight exceeds a threshold amount while the actuator is moving the component, to provide an indication to a user that contact with an obstruction has been made if the change in the weight exceeds the threshold amount, and to allow the user to change a value of the threshold amount.

33. (canceled)

34. The patient support apparatus of claim 32 wherein the control panel is adapted to allow the user to select at least three different values for the threshold amount, wherein the at least three different values correspond to high, medium, and low sensitivity levels for detecting contact with the obstruction.

35. The patient support apparatus of claim 32 wherein the controller is adapted to determine a location of the contact with the obstruction by calculating a center of gravity of the change in the weight.

36. The patient support apparatus of claim 35 wherein the controller is further adapted to use a first value for the threshold amount if the location of the contact falls within a first area and to use a second value for the threshold amount if the location of the contact falls within a second area, wherein the first value is different from the second value and the first area is different from the second area.

37. The patient support apparatus of claim 36 further comprising a plurality of siderails moveable between raised and lowered positions, wherein the controller is further adapted to define a boundary of the first area based on a position of at least one of the siderails.

38-40. (canceled)

41. The patient support apparatus of claim 32 wherein the controller is further adapted to automatically stop movement of the component when contact is detected; to determine a maximum weight change reading while the component is moving and a residual weight change reading when the controller automatically stops movement of the component; and to use the maximum weight change reading and the residual weight change reading to evaluate whether to provide the indication to the user that contact with the obstruction has been made.

42-44. (canceled)

45. The patient support apparatus of claim 41 wherein the controller is further adapted to determine a rate at which the weight changes while the component is moving, and to use the rate at which the weight changes to evaluate whether to provide the indication to the user that contact with the obstruction has been made.

46-59. (canceled)

60. A patient support apparatus comprising:
a support deck adapted to support a patient thereon;
a frame adapted to support the support deck;
a plurality of force sensors adapted to detect a weight supported by the frame;
an actuator adapted to move a component of the patient support apparatus;
a control panel including a movement control, wherein the movement control is adapted to drive the actuator when the movement control is activated; and
a controller adapted to detect a location of a change in weight on the patient support apparatus while the actuator is moving the component, to provide an indication to a user that contact with an obstruction has been made if the change in the weight exceeds a first threshold amount and the change in weight occurs in a first area of the patient support apparatus, and to provide the indication to the user that contact with the obstruction has been made if the change in weight exceeds a second threshold amount and the change in weight occurs in a second area different from the first area, wherein the first threshold amount is different from the second threshold amount.

61-62. (canceled)

63. The patient support apparatus of claim 60 wherein the control panel is adapted to allow the user to select at least three different values for both the first and second threshold amounts, wherein the at least three different values correspond to high, medium, and low sensitivity levels for detecting contact with the obstruction.

64-68. (canceled)

69. The patient support apparatus of claim 60 wherein the controller is further adapted to automatically stop movement of the component when contact is detected; to determine a maximum weight change reading while the component is moving and a residual weight change reading when the controller automatically stops movement of the component; and to use the maximum weight change reading and the residual weight change reading to evaluate whether to provide the indication to the user that contact with the obstruction has been made.

70-72. (canceled)

73. The patient support apparatus of claim 69 wherein the controller is further adapted to determine a rate at which the weight changes while the component is moving and to use the rate at which the weight changes to evaluate whether to provide the indication to the user that contact with the obstruction has been made.

74-76. (canceled)

77. The patient support apparatus of claim 60 further comprising a display, and wherein the controller is adapted to display the location of the contact on the display.

78-82. (canceled)

83. The patient support apparatus of claim 60 wherein the control panel includes a touch screen display and the controller is adapted to display a recovery control on the touch screen display in response to the contact with the obstruction, wherein the recovery control is adapted to move the component out of contact with the obstruction when the recovery control is activated; and wherein the controller is further adapted to detect if a second change in the weight exceeds the first threshold amount if detected in the first area or exceeds the second threshold amount if detected in the second area while the recovery control is activated, and to not provide the indication to the user if the second change in the weight is substantially equal to, but of opposite sign of, the change in weight.

84-179. (canceled)

180. A patient support apparatus comprising:
a support deck adapted to support a patient thereon;
a frame adapted to support the support deck;
a plurality of force sensors adapted to detect a weight supported by the frame;
an actuator adapted to move a component of the patient support apparatus;
a control panel including a movement control, wherein the movement control is adapted to drive the actuator when the movement control is activated; and
a controller adapted to automatically stop movement of the component in response to contact with an obstruction, to record a first change in weight during movement of the component and a residual change in weight when movement of the component is automatically stopped, and to use the residual change in weight to determine if actual contact with the obstruction has occurred or a false detection of contact with the obstruction has occurred.

181. The patient support apparatus of claim 180 wherein the controller is adapted to determine if actual contact with the obstruction has occurred by detecting if the first change in the weight exceeds a threshold amount while the actuator is moving the component.

182. The patient support apparatus of claim 181 wherein the controller is further adapted to determine if actual contact with the obstruction has occurred by analyzing a rate of the first change in weight while the component is moving.

183. The patient support apparatus of claim 181 wherein the controller is further adapted to record instances in which actual contact has not occurred, to analyze the instances, and to suggest a different threshold amount to a user in order to reduce a number of the instances in which actual contact has not occurred but the controller has automatically stopped movement of the component.

184-186. (canceled)

187. The patient support apparatus of claim 181 wherein the controller is adapted to determine a location of the contact with the obstruction by calculating a center of gravity of the first change in the weight, to use a first value for the threshold amount if the location of the contact falls within a first area and to use a second value for the threshold amount if the location of the contact falls within a second area, wherein the first value is different from the second value and the first area is different from the second area.

188-197. (canceled)

198. The patient support apparatus of claim 180 wherein the controller is further adapted to display a graphic representation of the patient support apparatus on a display, the graphic representation including an arrow indicating which direction the component of the patient support apparatus should be moved in order to move away from the obstruction.

199-212. (canceled)
